SEQUENCE LISTING (56 sequences)

SEQ ID NO: 1 - protein, 450 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 452 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4-10 - skipped (7 entries)

SEQ ID NO: 11 - protein, 1057 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 12 - protein, 588 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - protein, 1057 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 14 - protein, 588 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 15 - protein, 1057 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 16 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 17 - protein, 475 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 18 - protein, 588 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 19 - protein, 973 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 20 - protein, 210 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 352 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 22 - protein, 98 aa, synthetic construct
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 23 - protein, 330 aa, synthetic construct [sequence omitted]

SEQ ID NO: 24 - protein, 5 aa, synthetic construct
DYVIN

SEQ ID NO: 25 - protein, 17 aa, synthetic construct
EIYPGSGTNYYNEKFKA

SEQ ID NO: 26 - protein, 11 aa, synthetic construct
RGRYGLYAMDY

SEQ ID NO: 27 - protein, 11 aa, synthetic construct
RASQDISNYLN

SEQ ID NO: 28 - protein, 7 aa, synthetic construct
YTSRLHS

SEQ ID NO: 29 - protein, 9 aa, synthetic construct
QQGNTRPWT

SEQ ID NO: 30 - protein, 5 aa, synthetic construct
GGGGS

SEQ ID NO: 31 - protein, 20 aa, synthetic construct
GGGGSGGGGSGGGGSGGGGS

SEQ ID NO: 32 - protein, 7 aa, synthetic construct
GGGSGGG

SEQ ID NO: 33 - protein, 10 aa, synthetic construct
GGSGGGGSGG

SEQ ID NO: 34 - protein, 18 aa, synthetic construct
KESGSVSSEQLAQFRSLD

SEQ ID NO: 35 - protein, 14 aa, synthetic construct
EGKSSGSGSESKST

SEQ ID NO: 36 - protein, 592 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 37 - protein, 184 aa, synthetic construct
REGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 38 - protein, 133 aa, synthetic construct [sequence omitted]

SEQ ID NO: 39 - protein, 1057 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 40 - protein, 973 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGGGGSGGGGSGGGGSGGGGSREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 41-48 - skipped (8 entries)

SEQ ID NO: 49 - protein, 96 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSP

SEQ ID NO: 50 - protein, 304 aa, synthetic construct
MSSTLPALLCVGLCLSQRISAQQQTLPKPFIWAEPHFMVPKEKQVTICCQGNYGAVEYQLHFEGSLFAVDRPKPPERINKVQFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 51 - protein, 276 aa, synthetic construct
MAWRALHPLLLLLLLFPGSQAQSKAQVLQSVAGQTLTVRCQYPPTGSLYEKKGWCKEASALVCIRLVTSSKPRTMAWTSRFTIWDDPDAGFFTVTMTDLREEDSGHYWCRIYRPSDNSVSKSVRFYLVVSPASASTQTSWTPRDLVSSQTQTQSCVPPTAGARQAPESPSTIPVPSQPQNSTLRPGPAAPIALVPVFCGLLVAKSLVLSALLVWWGDIWWKTMMELRSLDTQKATCHLQQVTDLPWTSVSSPVEREILYHTVARTKISDDDDEHTL

SEQ ID NO: 52 - protein, 201 aa, synthetic construct
MAWMLLLILIMVHPGSCALWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLGAGTVLLLRAGFYAVSFLSVAVGSTVYYQGKCLTWKGPRRQLPAVVPAPLPPPCGSSAHLLPPVPGG

SEQ ID NO: 53 - protein, 216 aa, synthetic construct
MGWIRGRRSRHSWEMSEFHNYNLDLKKSDFSTRWQKQRCPVVKSKCRENASPFFFCCFIAVAMGIRFIIMVTIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 54 - protein, 336 aa, synthetic construct
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 55 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 56 - protein, 335 aa, synthetic construct
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

(19) United States
(12) Patent Application Publication
(10) Pub. No.: US 2026/0327283 A1
(43) Pub. Date: Oct. 22, 2026

(54) Multispecific antigen binding proteins for stimulating NK cells and use thereof

(71) Applicant: Avidicure IP B.V., Amsterdam (NL)

(72) Inventor: Robert Heinz Edward Friesen, Breukelen (NL)

(73) Assignee: Avidicure IP B.V., Amsterdam (NL)

(21) Appl. No.: 19/100,966

(22) PCT Filed: Sep. 15, 2023
(86) PCT No.: PCT/EP2023/075438

§ 371 Date: Feb. 4, 2025

(30) Foreign Application Priority Data
Sep. 15, 2022 (EP) 22195902.6
Sep. 15, 2022 (EP) 22195904.2

(51) Int. Cl. C07K 14/54; A61K 35/17; A61K 38/00; A61K 39/00; A61P 35/00; C07K 14/525; C07K 16/28; C12N 5/0783

(52) U.S. Cl. CPC C07K 14/54; A61K 35/17; A61P 35/00; C07K 14/525; C07K 16/2803; C07K 16/2827; C07K 16/283; C07K 16/2878; C07K 16/2896; C12N 5/0646; A61K 38/00; A61K 2039/505; C07K 2319/30; C12N 2501/2321; C12N 2501/25; C12N 2501/50; C12N 2501/51; C12N 2501/599; C12N 2502/30

(57) ABSTRACT

The present invention relates to multispecific antigen binding proteins that comprise an natural killer (NK) cell-activating cytokines and a region that has affinity for a surface antigen expressed on NK cells, e.g CD16A. The NK cell-activating cytokine preferably is at least one of an interleukin 21 receptor (IL21R) agonist and a 4-1BB agonist. The multispecific antigen binding protein can further comprise an antigen binding region that specifically binds an NK cell activating receptor, such as NKp46, NKp44, NKp30, NKG2D, DNAM1 and CD16A. The multispecific antigen binding proteins of the invention can be used ex or in vivo for expanding and activating NK cells. The invention further relates to the use of such multispecific antigen binding proteins and/or ex vivo expanded NK cells in the treatment of cancer.

### FIELD OF THE INVENTION

The present invention relates to the field of medicine, in particular to the fields of oncology, immunology and immunotherapy of tumors. Specifically, the invention relates to multispecific antigen binding proteins that stimulate and activate NK cells so that they can lyse target cells, such as tumor cells. The invention further relates to the use of such multispecific antigen binding proteins in the treatment of cancer.

### BACKGROUND OF THE INVENTION

Immunotherapy of cancer is revolutionizing the treatment of cancers. Cancer immunotherapies are desirable because they are highly specific and can facilitate destruction of a tumor by inducing the recognition and elimination of tumor cells by the patient's own immune system. Recent advances have focused on generating or unleashing tumor antigen-specific T cell responses. They were based on the use of immune checkpoint inhibitors targeting inhibitory pathways, or bispecific T cell engagers and chimeric antigen receptor (CAR) T cells targeting a tumor antigen. Despite these outstanding breakthroughs, the clinical benefit has been limited to a subset of patients and certain tumor types, highlighting the need for alternative strategies.

One such alternative approach is to exploit the antitumor activity of natural killer (NK) cells. NK cells are a component of the innate immune system and make up approximately 15% of circulating lymphocytes. NK cells infiltrate virtually all tissues and were originally characterized by their ability to kill tumor cells effectively without the need for prior sensitization. NK cells provide an efficient immunosurveillance mechanism by which undesired cells such as tumor cells or virally-infected cells can be eliminated. The biological properties of NK cells include the expression of surface antigens including CD16, CD56 and/or CD57, the absence of the α/β or γ/δ TCR complex on the cell surface; the ability to recognize and kill cells that fail to express “self” MHC/HLA antigens by the activation of specific cytolytic enzymes, the ability to kill tumor cells or other diseased cells that express stress ligands for NK activating receptors, and the ability to release protein molecules called cytokines that stimulate or inhibit the immune response. Activated NK cells kill target cells by means similar to cytotoxic T cells, i.e., via cytolytic granules that contain perforin and granzymes as well as via death receptor pathways. Activated NK cells also secrete inflammatory cytokines such as IFN-γ and chemokines that promote the recruitment of other leukocytes to the target tissue. NK cells respond to signals through a variety of activating and inhibitory receptors on their surface. For example, when NK cells encounter healthy self-cells, their activity is inhibited through activation of the killer-cell immunoglobulin-like receptors (KIRs). Alternatively, when NK cells encounter foreign cells or cancer cells, they are activated via their activating receptors (e.g., NKG2D, NCRs, DNAM1). NK cells are also activated by the constant region of some immunoglobulins through CD 16 receptors on their surface. The overall sensitivity of NK cells to activation depends on the sum of stimulatory and inhibitory signals.

Strategies based on the recruitment of cytotoxic NK cells are currently being developed. It is expected that treatments based on NK cells are likely to be safer than T-cell treatments, given the absence of graft-versus-host reactions in patients receiving infusions of allogeneic NK cells. Furthermore, unlike chimeric antigen receptor (CAR) T cells, the administration of allogenic CAR-engineered NK cells is not associated with the development of neurotoxicity, cytokine release syndrome (CRS), or graft-versus-host disease, nor does CAR-NK cell infusion increase circulating inflammatory cytokine concentrations above baseline levels. NK cell-based immunotherapies may be less likely to cause these adverse events because the spectrum of cytokines produced by these cells is different from that secreted by T cells. Moreover, NK cells have a frequency only about one tenth that of T cells, which lower frequency ensures a more moderate release of cytokines, limiting the risk of CRS in patients treated with NK cell-targeting multispecific antibodies.

More recently, multifunctional antibodies called natural killer cell engagers (NKCEs) have been developed, which simultaneously target tumor-associated antigens (TAAs) and activate receptors on endogenous NK cells. NKCEs are designed to strengthen the interaction between the NK cell and targeted tumor cell and to increase NK cell effector functions towards the tumor cell. A number of NKCEs that are currently in development for clinical application is reviewed by Demaria et al. (Eur. J. Immunol. 2021. 51:1934-1942).

There remains a need in the art for improved multispecific antigen binding proteins for induction of NK cells with new and additional functionalities, particularly those that provide therapeutic advantages over existing NKCEs.

### SUMMARY OF THE INVENTION

In a first aspect, the invention relates to a multispecific antigen binding protein comprising: a) an NK cell-activating cytokine that is at least one of: i) an interleukin 21 receptor (IL21R) agonist; and, ii) a 4-1BB agonist; and, b) a region that has affinity for a surface antigen expressed on natural killer (NK) cells.

In one embodiment, the multispecific antigen binding protein is a multispecific antigen binding protein, wherein the IL21R agonist comprises or consist of an IL21 polypeptide or an agonistic antigen-binding region that specifically binds IL21R. In one embodiment, a multispecific antigen binding protein as described herein comprises an IL21R agonist that is an IL21 polypeptide comprising an amino acid sequence with at least 50, 55, 60, 65, 70, 75, 80, 85, 90, 95, 96, 97, 98, 99, or 100% sequence identity to SEQ ID NO: 38, and preferably having an IL21R agonist activity as defined herein, and/or preferably having an affinity for the IL21R as defined herein. In one embodiment, the IL21 polypeptide is an IL21 mutein that is modified to reduce or enhance affinity for IL21R, relative to a corresponding wild type IL21 polypeptide. For example, an IL21 mutein that has reduced affinity for IL21R, relative to a corresponding wild type IL21 polypeptide, can be an IL21 mutein that has a mutation in one or more amino acids selected from the group consisting of I16, I66, I8, K72, K73, K75, K77, L13, P78, Q12, Q19, R5, R65, R76, R9, S70, S80, V69 and Y23. In one embodiment, the multispecific antigen binding protein is a multispecific antigen binding protein that has an IL21R agonist-valency that is higher than one.

In one embodiment, the multispecific antigen binding protein is a multispecific antigen binding protein, wherein the 4-1BB agonist comprises or consist of at least one 4-1BB ligand (4-1BBL) extracellular domain (ECD) or at least one agonistic antigen-binding region that specifically binds 4-1BB. In one embodiment, a multispecific antigen binding protein as described herein comprises a 4-1BB agonist comprising at least one 4-1BBL ECD comprising an amino acid sequence with at least 50, 55, 60, 65, 70, 75, 80, 85, 90, 95, 96, 97, 98, 99, or 100% sequence identity to SEQ ID NO: 37, and preferably having an 4-1BB agonist activity as defined herein, and/or preferably having an affinity for 4-1BB as defined herein. In one embodiment, the 4-1BBL ECD is a mutein that is modified to reduce or enhance affinity for 4-1BB, relative to a corresponding wild type 4-1BBL ECD. In one embodiment, the 4-1BB agonist comprises or consist of a fusion protein comprising three 4-1BBL ECD monomers fused together in a single polypeptide chain, and wherein, optionally, the three 4-1BBL ECD monomers are connected by polypeptide linkers. In one embodiment, the multispecific antigen binding protein is a multispecific antigen binding protein that has a 4-1BB agonist-valency that is higher than one.

In one embodiment, the multispecific antigen binding protein is a multispecific antigen binding protein comprising at least one IL21R agonist and at least one 4-1BB agonist.

In one embodiment, the multispecific antigen binding protein is a multispecific antigen binding protein further comprising an NK cell-activating cytokine selected from the group consisting of: an IL15 receptor agonist, an IL2 receptor agonist, a type I interferon (IFN-1) agonist, an IL12 receptor agonist and an IL18 receptor agonist.

In one embodiment, the multispecific antigen binding protein is a multispecific antigen binding protein wherein the region that has affinity for a surface antigen expressed on NK cells comprises or consists of an immunoglobulin Fc region. Preferably, the Fc region is a dimeric Fc region. In one embodiment, the Fc region is an Fc region that binds to CD16A. In one embodiment, the Fc region is an Fc region that is modified to reduce or enhance affinity for CD16A, relative to a corresponding wild-type Fc region. In one embodiment, the Fc region is an Fc region that is modified to reduce or enhance NK cell activation through CD16A binding, relative to a corresponding wild-type Fc region.

In one embodiment, the multispecific antigen binding protein further comprises: c) at least one antigen-binding region that specifically binds an NK cell activating receptor. In one embodiment, the antigen-binding region that specifically binds an NK cell activating receptor is an agonistic antigen-binding region that activates the NK cell receptor. Preferably, the antigen-binding region comprises at least one immunoglobulin variable region, more preferably, the immunoglobulin variable region comprises or consists of a Fab or an immunoglobulin single variable domain (ISVD). In one embodiment, the antigen-binding region is a human or humanized antigen-binding region.

In one embodiment, the multispecific antigen binding protein comprises two antigen-binding regions that specifically bind an NK cell activating receptor. The two antigen-binding regions can bind the same NK cell activating receptor or they can bind at least two different NK cell activating receptors. In one embodiment, the two antigen binding regions are identical.

In one embodiment, the multispecific antigen binding protein is a multispecific antigen binding protein wherein the NK cell activating receptor (that is bound by the antigen-binding region) is selected from the group consisting of NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-BB, OX40, CRTAM, CD27, PSGL1, CD96, CD100, CEACAM1, CD59, PD-L1, Tim3 and NTB-A.

In one embodiment, the multispecific antigen binding protein is a protein wherein the antigen-binding region comprises a combination of complementarity-determining regions (CDRs) CDR-H1, CDR-H2, CDR-H3, CDR-L1, CDR-L2 and CDR-L3 selected from the group consisting of: a) the CDR-H1 (SEQ ID NO: 24), CDR-H2 (SEQ ID NO: 25) and CDR-H3 (SEQ ID NO: 26) sequences as comprised in SEQ ID NO: 1, and the CDR-L1 (SEQ ID NO: 27), CDR-L2 (SEQ ID NO: 28) and CDR-L3 (SEQ ID NO: 29) sequences as comprised in SEQ ID NO: 2; and b) the CDR-H1, CDR-H2 and CDR-H3 sequences as comprised in SEQ ID NO: 3, and the CDR-L1, CDR-L2 and CDR-L3 sequences as comprised in SEQ ID NO: 20. Preferably, the multispecific antigen binding protein is a protein wherein the antigen-binding region comprises a combination of variable light (V_{L}) and variable heavy (V_{H}) domains selected from the group consisting of: a) the V_{H} sequence as comprised in SEQ ID NO: 1 and the V_{L} sequence as comprised in SEQ ID NO: 2; and b) the V_{H} sequence as comprised in SEQ ID NO: 3 and the V_{L} sequence as comprised in SEQ ID NO: 20.

In one embodiment, the multispecific antigen binding protein is a multispecific antigen binding protein wherein at least one NK cell-activating cytokine is conjugated to the region that has affinity for a surface antigen expressed on NK cells. In one embodiment, the at least one NK cell-activating cytokine forms a single polypeptide chain with at least one polypeptide chain of the region that has affinity for a surface antigen expressed on NK cells, and wherein optionally the NK cell-activating cytokine is linked to the polypeptide chain of the region that has affinity for a surface antigen expressed on NK cells through a flexible linker. Preferably, the single polypeptide chain comprises in an N- to C-terminal order: i) a first NK cell-activating cytokine, ii) optionally a first flexible linker; iii) the polypeptide chain of the region that has affinity for a surface antigen expressed on NK cells; iv) optionally a second flexible linker; and v) a second NK cell-activating cytokine; and wherein preferably, the first and second NK cell-activating cytokine are different NK cell-activating cytokines. Preferably, the at least one antigen-binding region that specifically binds an NK cell activating receptor is conjugated to the region that has affinity for a surface antigen expressed on NK cells. Preferably, at least one polypeptide chain in the at least one antigen-binding region that specifically binds an NK cell activating receptor forms a single polypeptide chain with at least one polypeptide chain of the region that has affinity for a surface antigen expressed on NK cells. Preferably, the single polypeptide chain comprises in an N- to C-terminal order: i) at least one polypeptide chain in the at least one antigen-binding region that specifically binds an NK cell activating receptor; ii) optionally a flexible linker; and iii) the region that has affinity for a surface antigen expressed on NK cells. In one embodiment, the region that has affinity for a surface antigen expressed on NK cells is a dimeric immunoglobulin Fc region, wherein each of the two polypeptide chains of the dimeric Fc region is linked to a CH1 domain, each of which CH1 domains is linked to an immunoglobulin variable region that specifically binds an NK cell activating receptor. Preferably, the two immunoglobulin variable regions bind the same NK cell activating receptor, or wherein the two immunoglobulin variable regions each bind a different NK cell activating receptor. In one embodiment, the protein comprises a dimeric immunoglobulin Fc region, wherein each of the two Fc polypeptide chains is operably linked to a Fab that specifically binds an NK cell activating receptor.

In one embodiment, the multispecific antigen binding protein is a multispecific antigen binding protein wherein at least one NK cell-activating cytokine, is conjugated to the at least one antigen-binding region that specifically binds an NK cell activating receptor, or to the region that has affinity for a surface antigen expressed on NK cells. Preferably, at least one of the NK cell-activating cytokines forms a single polypeptide chain with at least one of: i) at least one polypeptide chain in the at least one antigen-binding region that specifically binds an NK cell activating receptor; and, ii) at least one polypeptide chain in the region that has affinity for a surface antigen expressed on NK cells; wherein optionally, a flexible linker is present between the agonist and the at least one polypeptide chain in the region defined in i) or ii). In one embodiment, at least one of the NK cell-activating cytokines forms a single polypeptide chain with at least one of: i) a light chain in at least one of the two Fabs that specifically bind an NK cell activating receptor; and, ii) at least one of the two Fc chains in the dimeric immunoglobulin Fc region; wherein optionally, a flexible linker is present between the agonist and the light chain defined in i) or the Fc chain defined in ii). Preferably, at least one of the NK cell-activating cytokines is fused to at least one of: i) the N-terminus of the light chain in at least one of the two Fabs that specifically bind an NK cell activating receptor, optionally through a flexible linker; ii) the C-terminus of the light chain in at least one of the two Fabs that specifically bind an NK cell activating receptor, optionally through a flexible linker; iii) the N-terminus of the heavy chain in at least one of the two Fabs that specifically bind an NK cell activating receptor; and, iv) the C-terminus of the heavy chain in at least one of the two Fc chains in the dimeric immunoglobulin Fc region, optionally through a flexible linker. In one embodiment, at least one of the NK cell-activating cytokines is present on at least one or on both sides of the immunoglobulin structure.

In one embodiment, the multispecific antigen binding protein is a multispecific antigen binding protein wherein the multispecific antigen binding protein is heterodimeric with respect to at least one of i) the antigen-binding regions that specifically bind an NK cell activating receptor; and ii) at least one fused NK cell-activating cytokine, and wherein the dimeric Fc region comprises different first and a second polypeptide chains comprising knob-into-hole modifications promoting association of the first and the second polypeptide chains of the Fc region.

In one embodiment, the multispecific antigen binding protein is a multispecific antigen binding protein that has at least one biological activity selected from: a) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are not brought into contact with the multispecific antigen binding protein; and, b) the multispecific antigen binding protein cause an increase in at least one NK cell activity selected from CD107a degranulation, CD107 or CD69 expression, IFNy production, NK proliferation and NK cell cytotoxicity, whereby preferably, the increase is at least a factor 0.1 higher as compared to the increase achieved with the same effector:target cell ratio, with the same NK cells and target cells that are brought into contact with a conventional human IgG1 monoclonal antibody that has same NK cell activating receptor-specific antigen-binding regions as the multispecific antigen binding protein.

In one embodiment, the multispecific antigen binding protein is a multispecific antigen binding protein wherein ex vivo expansion of donor NK cells by co-culturing with the multispecific antigen binding protein as described herein, produces a population of expanded NK cells having one or more features selected from: a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells); b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of NK cells obtained upon ex vivo expansion in the presence of FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on expanded NK cells obtained by ex vivo expansion by co-culturing with irradiated FC21 feeder cells; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold of the secretion of the cytokine by expanded NK cells obtained by ex vivo expansion by co-culturing with irradiated FC21 feeder cells; and, e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of expanded NK cells obtained by ex vivo expansion by co-culturing with irradiated FC21 feeder cells.

In one embodiment, the ex vivo expansion of donor NK cells by co-culturing with the multispecific antigen binding protein as described herein and by co-stimulation with irradiated tumor cells or with tumor cells, produces a population of expanded NK cells having one or more features selected from: a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells; b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold; and, e) the cytotoxicity of the expanded NK cells is at least 0.001, 0.002, 0.005, 0.01, 0.02, 0.05, 0.1, 0.2, 0.5, 1.0, 2.0 or 5.0 fold.

In a second aspect, the invention pertains to a pharmaceutical composition comprising a multispecific antigen binding protein as described herein, and a pharmaceutically acceptable carrier.

In a third aspect, the invention relates to an ex vivo method for expansion of NK cells, the method comprising the step of contacting an NK cell with a multispecific antigen binding protein as described herein, or with the pharmaceutical composition comprising the protein, wherein preferably, the expanded NK cells have one or more features selected from: a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells); b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and, e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells.

Additionally, the invention relates to an ex vivo method for expansion of NK cells, the method comprising the step of contacting an NK cell with a multispecific antigen binding protein as described herein, or with the pharmaceutical composition comprising the protein and with with irradiated tumor cells or with tumor cells, wherein preferably, the expanded NK cells have one or more features selected from: a) the fold expansion of the expanded NK cells is at least 0.001, 0.002, 0.005, 0.01, 0.02, 0.05, 0.1, 0.2, 0.5, 1.0, 2.0, or 5.0 fold; b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold; and, e) the cytotoxicity of the expanded NK cells is at least 0.001, 0.002, 0.005, 0.01, 0.02, 0.05, 0.1, 0.2, 0.5, 1.0, 2.0, or 5.0 fold. In one embodiment, the multispecific antigen binding protein causes an increase in Glut1, Glut3, CD71, and/or CD98 expression, mitochondrial mass, glycolytic rate, ratio of glycolytic rate to oxidative phosphorylation rate, metabolic fuel flexibility among glucose, glutamine, and/or fatty acids, whereby preferably, the increase is at least a factor 0.05 higher as compared to the increase achieved with the same effector:target cell ratio, with the same NK cells and target cells that are not brought into contact with the multispecific antigen binding protein; b) the multispecific antigen binding protein causes an increase in at least one NK cell activity selected from Glut1, Glut3, CD71, or CD98 expression, mitochondrial mass, glycolytic rate, ratio of glycolytic rate to oxidative phosphorylation rate, metabolic fuel flexibility among glucose, glutamine, and fatty acids, whereby preferably, the increase is at least a factor 0.05 higher as compared to the increase achieved with the same effector:target cell ratio, with the same NK cells and target cells that are brought into contact with a conventional human IgG1 monoclonal antibody that has the same TAA-specific antigen-binding regions as the multispecific antigen binding protein.

In one embodiment ex vivo expansion of donor NK cells by co-culturing with the multispecific antigen binding protein as described herein, produces a population of expanded NK cells having one or more features selected from: a) mitochondrial mass or the expression of at least one nutrient transporter of Glut1, Glut3, CD71, or CD98 that is at least 0.001, 0.002, 0.005, 0.01, 0.02, 0.05, 0.1, 0.2, 0.5, 1.0, 2.0, 5.0, 10.0, or 20 fold the mitochondrial mass or expression of the nutrient transporter on expanded NK cells obtained by ex vivo expansion by co-culturing with irradiated K562 feeder cells modified to express membrane bound IL-21 (mbIL-21) and 4-1BB ligand (FC21 feeder cells); b) the glycolytic rate is at least 0.01, 0.02, 0.05, 0.1, 0.2, 0.5, 1.0, 2.0, 5.0, 10.0, or 20.0 fold the glycolytic rate of expanded NK cells obtained by ex vivo expansion by co-culturing with irradiated K562 feeder cells modified to express membrane bound IL-21 (mbIL-21) and 4-1BB ligand (FC21 feeder cells); c) the ratio of glycolytic rate to oxidative phosphorylation (OxPhos) rate is at least 0.01, 0.02, 0.05, 0.1, 0.2, 0.5, 1.0, 2.0, 5.0, 10.0, or 20.0 fold the ratio of glycolytic rate to OxPhos rate of expanded NK cells obtained by ex vivo expansion by co-culturing with irradiated K562 feeder cells modified to express membrane bound IL-21 (mbIL-21) and 4-1BB ligand (FC21 feeder cells); d) metabolic fuel flexibility among glucose, glutamine, and fatty acids is at least 0.01, 0.02, 0.05, 0.1, 0.2, 0.5, 1.0, 2.0, 5.0, or 10.0 fold the metabolic fuel flexibility among glucose, glutamine, and fatty acids of expanded NK cells obtained by ex vivo expansion by co-culturing with irradiated K562 feeder cells modified to express membrane bound IL-21 (mbIL-21) and 4-1BB ligand (FC21 feeder cells).

In a fourth aspect, the invention pertains to a multispecific antigen binding protein as described herein, pharmaceutical composition comprising the protein, or an ex vivo expanded NK cell obtained in the above method, optionally in combination with the multispecific antigen binding protein, for use as a medicament.

In a fifth aspect, the invention relates to a multispecific antigen binding protein as described herein, a pharmaceutical composition comprising the protein, or an ex vivo expanded NK cell obtained in the above method, optionally in combination with the multispecific antigen binding protein, for use in the treatment of a cancer. In one embodiment, the invention relates to a multispecific antigen binding protein as described herein, or a pharmaceutical composition comprising the protein for use in the treatment of a cancer, wherein the multispecific antigen binding protein or the composition is used in combination with an adoptive transfer of immune cells, wherein preferably the immune cells are selected from T cells and NK cells.

In a sixth aspect, the invention relates to method for enhancing anti-tumor activity of an NK cell in a subject, the method comprising the step of administering to the subject a multispecific antigen binding protein as described herein, pharmaceutical composition comprising the protein, an ex vivo expanded NK cell obtained in the above method, optionally in combination with the multispecific antigen binding protein, or a combination of the multispecific antigen binding protein and an immune cell selected from T cells and NK cells. In one embodiment of the method, the subject has cancer. In one embodiment, the invention relates to the multispecific antigen binding protein, the composition comprising the protein, or the ex vivo expanded NK cell, optionally in combination with the multispecific antigen binding protein, for the above uses, wherein at least one of: a) the multispecific antigen binding protein and/or the ex vivo expanded NK cell is administered as a neoadjuvant therapy before a primary therapy comprising at least one of surgery and radiation therapy of the cancer; and, b) the multispecific antigen binding protein and/or the ex vivo expanded NK cell is administered as an adjuvant therapy after a primary therapy comprising at least one of surgery and radiation therapy of the cancer.

In a seventh aspect, the invention relates to a nucleic acid molecule comprising one or more nucleotide sequences encoding a polypeptide chain of a multispecific antigen binding protein as described herein. Preferably, the nucleic acid molecule is a nucleic acid molecule wherein the one or more nucleotide sequences are operably linked to regulatory sequences for expression of the one or more polypeptide chains in a host cell.

In an eighth aspect, the invention relates to a host cell comprising a nucleic acid molecule as defined above.

In a nineth aspect, the invention relates to a method for producing a multispecific antigen binding protein as described herein, the method comprising culturing a host cell as defined above, such that one or more nucleotide sequences are expressed, and the multispecific antigen binding protein is produced. Preferably, the method further comprises the steps of: recovery of the multispecific antigen binding protein, and, optionally, formulation of the multispecific antigen binding protein with a pharmaceutically acceptable carrier.

### DESCRIPTION OF THE INVENTION

### Definitions

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

“A,” “an,” and “the”: these singular form terms include plural referents unless the content clearly dictates otherwise. The indefinite article “a” or “an” thus usually means “at least one”. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods. Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

“And/or”: The term “and/or” refers to a situation wherein one or more of the stated cases may occur, alone or in combination with at least one of the stated cases, up to with all of the stated cases.

“Comprising”: this term is construed as being inclusive and open ended, and not exclusive. Specifically, the term and variations thereof mean the specified features, steps or components are included. These terms are not to be interpreted to exclude the presence of other features, steps or components.

Exemplary”: this term means “serving as an example, instance, or illustration,” and should not be construed as excluding other configurations disclosed herein.

As used herein “cancer” and “cancerous”, refer to or describe the physiological condition in mammals that is typically characterized by unregulated cell growth. Cancer is also referred to as malignant neoplasm.

As used herein, “in combination with” is intended to refer to all forms of administration that provide a first drug together with a further (second, third) drug. The drugs may be administered simultaneous, separate or sequential and in any order. Drugs administered in combination have biological activity in the subject to which the drugs are delivered.

As used herein “simultaneous” administration refers to administration of more than one drug at the same time, but not necessarily via the same route of administration or in the form of one combined formulation. For example, one drug may be provided orally whereas the other drug may be provided intravenously during a patient's visit to a hospital. Separate includes the administration of the drugs in separate form and/or at separate moments in time, but again, not necessarily via the same route of administration. Sequentially indicates that the administration of a first drug is followed, immediately or in time, by the administration of the second drug.

A used herein “compositions”, “products” or “combinations” useful in the methods of the present disclosure include those suitable for various routes of administration, including, but not limited to, intravenous, subcutaneous, intradermal, subdermal, intranodal, intratumoral, intramuscular, intraperitoneal, oral, nasal, topical (including buccal and sublingual), rectal, vaginal, aerosol and/or parenteral or mucosal application. The compositions, formulations, and products according to the disclosure invention normally comprise the drugs (alone or in combination) and one or more suitable pharmaceutically acceptable excipients.

As used herein, “an effective amount” is meant the amount of an agent required to ameliorate the symptoms of a disease relative to an untreated patient. The effective amount of active agent(s) used to practice the present invention for therapeutic treatment of a cancer varies depending upon the manner of administration, the age, body weight, and general health of the subject. Ultimately, the attending physician or veterinarian will decide the appropriate amount and dosage regimen. Such amount is referred to as an “effective” amount. Thus, in connection with the administration of a drug which, in the context of the current disclosure, is “effective against” a disease or condition indicates that administration in a clinically appropriate manner results in a beneficial effect for at least a statistically significant fraction of patients, such as an improvement of symptoms, a cure, a reduction in at least one disease sign or symptom, extension of life, improvement in quality of life, or other effect generally recognized as positive by medical doctors familiar with treating the particular type of disease or condition.

As used herein, the phrase “NK cells” refers to a sub-population of lymphocytes that is involved in innate immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to recognize and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (2000, edited by Campbell K S and Colonna M). Humana Press, pp. 219-238).

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods. The terms “sequence identity” or “sequence similarity” means that two (poly) peptide or two nucleotide sequences, when optimally aligned, preferably over the entire length (of at least the shortest sequence in the comparison) and maximizing the number of matches and minimizes the number of gaps such as by the programs ClustalW (1.83), GAP or BESTFIT using default parameters, share at least a certain percentage of sequence identity as defined elsewhere herein. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). A preferred multiple alignment program for aligning protein sequences of the invention is ClustalW (1.83) using a blosum matrix and default settings (Gap opening penalty: 10; Gap extension penalty: 0.05). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or using open source software, such as the program “needle” (using the global Needleman Wunsch algorithm) or “water” (using the local Smith Waterman algorithm) in EmbossWIN version 2.10.0, using the same parameters as for GAP above, or using the default settings (both for ‘needle’ and for ‘water’ and both for protein and for DNA alignments, the default Gap opening penalty is 10.0 and the default gap extension penalty is 0.5; default scoring matrices are Blosum62 for proteins and DNAFull for DNA). When sequences have a substantially different overall lengths, local alignments, such as those using the Smith Waterman algorithm, are preferred. Alternatively, percentage similarity or identity may be determined by searching against public databases, using algorithms such as FASTA, BLAST, etc.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. Examples of classes of amino acid residues for conservative substitutions are given in the Tables below.

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes.

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues.

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, W, and |
| | Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, P and T |
| Flexible residues | Q, T, K, S, G, P, D, E, and R |

The term “agent” refers generally to any entity which is normally not present or not present at the levels being administered to a cell, tissue or subject. An agent can be a compound or a composition. An agent can e.g. be selected from the group consisting of: polynucleotides, polypeptides, small molecules, (multispecific) antigen binding proteins, such as antibodies and functional fragments thereof.

The term “antigen-binding domain” or “antigen-binding region” refers to the portion of an antigen-binding protein that is capable of specifically binding to an antigen or epitope. In one embodiment, the antigen-binding region is an immunoglobulin-derived antigen-binding region, e.g. comprising both an antibody light chain variable region (V_{L}) and an antibody heavy chain variable region (V_{H}). Examples of such antigen-binding regions include single-chain Fv (scFv), single-chain antibody, Fv, single-chain Fv2 (scFv2), Fab, and Fab′. In one embodiment, the antigen-binding region is an immunoglobulin-derived antigen-binding region from a single domain antibody consisting only of heavy chains and devoid of light chains as are known e.g. from camelids, wherein the antigen-binding site is present on, and formed by, the single variable domain (also referred to as an “immunoglobulin single variable domain” or “ISVD”). Examples of such ISVDs include the single variable domains of camelid heavy chain antibodies (V_{H}Hs), also referred to as nanobodies, domain antibodies (dAbs), and single domains derived from shark antibodies (IgNAR domains). In other embodiments, an antigen-binding region comprises a non-immunoglobulin-derived domain capable of specifically binding to an antigen or epitope, such as darpins; affilins; antikalins, etc.

The term “antibody” herein is used in the broadest sense and specifically includes full-length monoclonal antibodies, polyclonal antibodies, multispecific antibodies (e.g. bispecific antibodies), and antibody fragments and derivatives, so long as they exhibit the desired biological and/or immunological activity. Various techniques relevant to the production of antibodies are provided in, e.g., Harlow, et al. Antibodies: A Laboratory Manual, Cold Spring Harbor Laboratory Press, Cold Spring Harbor, N.Y., (1988). An antibody can be human and/or humanized. “Humanized” forms of non-human (e.g., rodent) antibodies are chimeric antibodies that contain minimal sequence derived from the non-human antibody.

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or m (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” comprises a portion of a full-length antibody, e.g. the antigen-binding or variable regions thereof. Examples of antibody fragments include Fab, Fab′, F(ab)2, F(ab′)2, F(ab)s, Fv (typically the V_{H} and V_{L} domains of a single arm of an antibody), single-chain Fv (scFv), dsFv, Fd fragments (typically the V_{H} and CH1 domain), and dAb (typically a V_{H} domain) fragments; V_{H}, V_{L}, V_{H}H, and V-NAR domains; minibodies, diabodies, triabodies, tetrabodies, and kappa bodies (see, e.g. Ill et al. Protein Eng 1997; 10:949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, N.Y., pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046. Diabodies are antibody fragments with two antigen-binding sites that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Various types of antibody fragments have been described or reviewed in, e.g. Heiliger and Hudson, Nat Biotechnol 2005; 23, 1126-1136; WO2005/040219, US20050238646 and US20020161201. Antibody fragments can be made by various techniques, including but not limited to proteolytic digestion of an intact antibody as well as production by recombinant host cells (e.g. CHO, E. coli or phage), as described herein.

The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, N.Y. (1988); Hammerling et al., in: “Monoclonal Antibodies and T-Cell Hybridomas,” Elsevier, N.Y. (1981), pp. 563-681 (both of which are incorporated herein by reference in their entireties).

The term “monospecific” antibody as used herein denotes that the antibody-part of a multispecific antigen binding protein as described herein, has one or more antigen-binding sites each of which bind to the same epitope of the same antigen. The term “bispecific” means that the antibody-part of a multispecific antigen binding protein as described herein, has at least two antigen-binding sites that are able to specifically bind to at least two distinct antigenic determinants. Typically, a bispecific antigen binding molecule comprises two antigen-binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” or “valency” as used within the current application denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule.

An antibody immunologically reactive with a particular antigen can be generated by recombinant methods such as selection of libraries of recombinant antibodies in phage or similar vectors, see, e.g., Huse et al., Science 246:1275-1281 (1989); Ward et al., Nature 341:544-546 (1989); and Vaughan et al., Nature Biotech. 14:309-314 (1996), or by immunizing an animal with the antigen or with DNA encoding the antigen. Methods for producing and screening for specific antibodies using hybridoma technology are routine and well known in the art. In a non-limiting example, mice can be immunized with an antigen of interest or a cell expressing such an antigen. Once an immune response is detected, e.g., antibodies specific for the antigen are detected in the mouse serum, the mouse spleen is harvested and splenocytes isolated. The splenocytes are then fused by well-known techniques to any suitable myeloma cells. Hybridomas are selected and cloned by limiting dilution. The hybridoma clones are then assayed by methods known in the art for cells that secrete antibodies capable of binding the antigen. Ascites fluid, which generally contains high levels of antibodies, can be generated by inoculating mice intraperitoneally with positive hybridoma clones.

Typically, an immunoglobulin has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The sequences of the framework regions of different light or heavy chains are relatively conserved within a species. The framework region of an antibody, that is the combined framework regions of the constituent light and heavy chains, serves to position and align the CDRs in three-dimensional space.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991, Sequences of Proteins of Immunological Interest, 5th Ed. Public Health Service, National Institutes of Health, Bethesda, MD, USA) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, an FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The term “framework” or “FR” residues as used herein refers to the region of an antibody variable domain exclusive of those regions defined as CDRs. Each antibody variable domain framework can be further subdivided into the contiguous regions separated by the CDRs (FR1, FR2, FR3 and FR4).

The term “constant region” as defined herein refers to an antibody-derived constant region that is encoded by one of the light or heavy chain immunoglobulin constant region genes. By “constant light chain” or “light chain constant region” as used herein is meant the region of an antibody encoded by the kappa (Ck) or lambda (CA) light chains. The constant light chain typically comprises a single domain, and as defined herein refers to positions 108-214 of CK or CA, wherein numbering is according to the EU index (Kabat et al., 1991, supra).

The term “constant heavy chain” or “heavy chain constant region” as used herein refers to the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment”” or “Fab region” refers to an antibody fragment comprising a light chain fragment comprising a VL domain and a constant domain of a light chain (CL), and a VH domain and a first constant domain (CH1) of a heavy chain. Fab may refer to this region in isolation, or this region in the context of a polypeptide, multispecific antigen binding protein or antigen-binding region, or any other embodiments as outlined herein. Fab′ fragments differ from Fab fragments by the addition of a few residues at the carboxy terminus of the heavy chain CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-combining sites (two Fab fragments) and a part of the Fc region.

The term “single-chain Fv” or “scFv” as used herein refers to antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, N.Y., pp. 269-315 (1994).

“Scaffold antigen-binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008). In one aspect of the invention, a scaffold antigen-binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (V_{NAR} fragments), a human gamma-crystallin or ubiquitin (Affilin molecules); a Kunitz type domain of human protease inhibitors, microbodies such as the proteins from the knottin family, peptide aptamers and fibronectin (adnectin).

CTLA-4 (Cytotoxic T Lymphocyte-associated Antigen 4) is a CD28-family receptor expressed on mainly CD4⁺ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001).

Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size and are derived from lipocalins. For further details see Biochim Biophys Acta 1482:337-350 (2000), U.S. Pat. No. 7,250,297B1 and US20070224633.

An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 17, 455-462 (2004) and EP1641818A1.

Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23 (12), 1556-1561 (2005) and Expert Opinion on Investigational Drugs 16 (6), 909-917 (June 2007).

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33-residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100 (4), 1700-1705 (2003) and J. Mol. Biol. 369, 1015-1028 (2007) and US20040132028A1.

A single-domain antibody is an antibody fragment consisting of a single monomeric variable antibody domain. The first single variable domains were derived from the variable domain of the antibody heavy chain from camelids (nanobodies or V_{H}H fragments). Furthermore, the term single-variable domain antibody includes an autonomous human heavy chain variable domain (aVH) or V_{NAR} fragments derived from sharks.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataBI and conotoxin and knottins. The microproteins have a loop which can be engineered to include up to 25 amino acids without affecting the overall fold of the microprotein. For further details of engineered knottin domains, see WO2008098796.

The term “Fv” or “Fv fragment” or “Fv region” as used herein refers to a polypeptide that comprises the V_{H} and V_{L} domains of a single antibody.

The term “Fc” or “Fc region”, as used herein refers to the polypeptide comprising the constant region of an antibody excluding the first constant region immunoglobulin domain. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides herein include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species. Fc thus refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cγ2 (CH2) and Cγ 3 (CH3) and the hinge between Cγ 1 and Cγ 2. Although the boundaries of the Fc region may vary, the human IgG heavy chain Fc region is usually defined to comprise residues C226, P230 or A231 to its carboxyl-terminus, wherein the numbering is according to the EU index. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc region, and the hole modification comprises the amino acid substitutions T366S, L368A and Y407V in the other one of the two subunits of the Fc region. In a further specific embodiment, the subunit of the Fc region comprising the knob modification additionally comprises the amino acid substitution S354C, and the subunit of the Fc region comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)). The numbering is according to EU index of Kabat et al, Sequences of Proteins of Immunological Interest, 5th Ed. Public Health Service, National Institutes of Health, Bethesda, MD, 1991.

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16A), FcyRI (CD64), FcyRIIa (CD32), and FcaRI (CD89). A particular activating Fc receptor is human FcγRIIIa (see UniProt accession no. P08637, version 141), also referred to as CD16 or CD16A. In humans, CD16 consists of two isoforms, CD16A and CD16B, encoded by two highly homologous genes. CD16A is a transmembrane protein expressed by lymphocytes and some monocytes, whereas CD16B is linked to the plasma membrane via a GPI anchor and primarily expressed by neutrophils. Therefore, when reference is made herein to CD16 in the context of expression on NK cells herein, usually CD16A is meant unless otherwise indicated.

By “variable region” as used herein is meant the region of an antibody that comprises one or more Ig domains substantially encoded by any of the V_{L} (including Vκ and Vλ) and/or V_{H} genes that make up the light chain (including κ and λ) and heavy chain immunoglobulin genetic loci respectively. A light or heavy chain variable region (V_{L} or V_{H}) comprise four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR,” as used herein refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (HI), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, MD (1991).) Hypervariable regions (HVRs) are also referred to as complementarity determining regions (CDRs), and these terms are used herein interchangeably in reference to portions of the variable region that form the antigen-binding regions. This particular region has been described by Kabat et al., U.S. Dept. of Health and Human Services, “Sequences of Proteins of Immunological Interest” (1983) and by Chothia et al., J. Mol. Biol. 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table A as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE A
CDR defintions¹

| CDR | Kabat | Chotia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table A is according to the numbering conventions set forth by Kabat et al. (see below).
²“AbM” with a lowercase “b” as used in Table A refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDRL2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

As used herein, the term “affinity matured” in the context of antigen-binding molecules (e.g., antibodies) refers to an antigen-binding molecule that is derived from a reference antigen-binding molecule, e.g., by mutation, binds to the same antigen, preferably binds to the same epitope, as the reference antibody; and has a higher affinity for the antigen than that of the reference antigen-binding molecule. Affinity maturation generally involves modification of one or more amino acid residues in one or more CDRs of the antigen-binding molecule. Typically, the affinity matured antigen-binding molecule binds to the same epitope as the initial reference antigen-binding molecule.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG1, IgG2, IgG3, IgG4, IgA1, and IgA2. The heavy chain constant domains that correspond to the different classes of immunoglobulins are called α, δ, ε, γ, and m respectively.

A “blocking” antibody or an “antagonist” antibody is one which inhibits or reduces biological activity of the antigen it binds. Preferred blocking antibodies or antagonist antibodies substantially or completely inhibit the biological activity of the antigen. An “agonist antibody”, as used herein, is an antibody which mimics at least one of the functional activities of a polypeptide of interest.

The term “specifically binds” refers to the number of different types of antigens or antigenic determinants to which a particular antigen-binding region or antigen-binding protein can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein. Alternatively, the affinity can also be expressed as the affinity constant (K_{A}), which is 1/K_{D}. Affinity can be determined in a manner known per se, depending on the specific combination of antigen-binding protein and antigen of interest. Avidity is herein understood to refer to the strength of binding of a target molecule with multiple binding sites by a larger complex of binding agents, i.e. the strength of binding of multivalent binding. Avidity is related to both the affinity between an antigenic determinant and its antigen-binding site on the antigen-binding protein and the valency, i.e. the number of binding sites present on the antigen-binding protein. Affinity, on the other hand refers to simple monovalent receptor ligand systems.

Typically, an antigen-binding region or a region that has affinity for a surface antigen expressed on NK cells of a multispecific antigen binding protein of the invention thereof will bind its target molecule (antigen) with a dissociation constant (K_{D}) of about 10⁻⁶ to 10⁻¹² M or less, and preferably 10⁻⁸ to 10⁻¹² M or less, and/or with a binding affinity of at least 10⁻⁶ M or 10⁻⁷ M, preferably at least 10⁻⁸ M, more preferably at least 10⁻⁹ M, such as at least 10⁻¹⁰, 10⁻¹¹, 10⁻¹² M or more. Any K_{D} value greater than 10⁻⁴ M (i.e. less than 100 μM) is generally considered to indicate non-specific binding. Thus, an antigen-binding region that “specifically binds” an antigen, is an antigen-binding domain that binds the antigen with a K_{D} value of no more than 10⁻⁴ M, as may be determined as herein described below. Preferably, an antigen-binding region or a region that has affinity for a surface antigen expressed on NK cells of a multispecific antigen binding protein of the invention will bind to the target molecule with an affinity less than 800, 400, 200, 100, 50, 10 or 5 nM, more preferably less than 1 nM, such as less than 500, 200, 100, 50, 10 or 5 pM. A variety of methods of measuring binding affinity are known in the art, any of which can be used for purposes of the present invention (see e.g. Harlow, et al., Antibodies: A Laboratory Manual, Cold Spring Harbor Laboratory Press, Cold Spring Harbor, N.Y., 1988), Coligan et al., eds. Current Protocols in Immunology, Greene Publishing Assoc, and Wiley Interscience, N.Y., (1992, 1993), and Muller, Meth. Enzymol. 92:589-601 (1983)). Specific illustrative embodiments are described in the following.

A “K_{D}” or “K_{D} value” can be measured by using an ELISA as described in the Examples herein or by using surface plasmon resonance assays using a BIAcore™-2000 or a BIAcore™ 3000 (BIAcore, Inc., Piscataway, NJ) at 25° C. with immobilized antigen CM5 chips at ~10-50 response units (RU). Briefly, carboxymethylated dextran biosensor chips (CM5, BIAcore Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, into 5 μg/ml (~0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of the antibody or Fab (0.78 nM to 500 nM) are injected in PBS with 0.05% Tween 20 (PBST) at 25° C. at a flow rate of approximately 25 μl/min. Association rates (kₒₙ) and dissociation rates (k_{off}) are calculated using a simple one-to-one Langmuir binding model (BIAcore Evaluation Software version 3.2) by simultaneous fitting the association and dissociation sensorgram. The equilibrium dissociation constant (K_{D}) is calculated as the ratio k_{off}/kₒₙ. See, e.g., Chen, Y., et al., (1999) J. Mol Biol 293:865-881. If the on-rate exceeds 10⁶ M⁻¹ S⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-Aminco spectrophotometer (ThermoSpectronic) with a stir red cuvette.

The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85%, at least 90%, and at least 95% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489 498 (1991); Studnicka et al., Prot. Eng. 7:805 814 (1994); Roguska et al., Proc. Natl. Acad. Sci. 91:969 973 (1994), and chain shuffling (U.S. Pat. No. 5,565,332), all of which are hereby incorporated by reference in their entireties.

One class of antigen-binding regions for use in the invention comprises immunoglobulin single variable domains (ISVDs) with an amino acid sequence that corresponds to the amino acid sequence of a naturally occurring single variable domain, but that has been “humanized”, i.e. by replacing one or more amino acid residues in the amino acid sequence of said naturally occurring single variable domain sequence by one or more of the amino acid residues that occur at the corresponding position(s) in a V_{H} domain from a conventional 4-chain antibody from a human being. This can be performed in a manner known per se, which will be clear to the skilled person, for example on the basis of the prior art on humanization including e.g. Jones et al. (Nature 321:522-525, 1986); Riechmann et al., (Nature 332:323-329, 1988); Presta (Curr. Op. Struct. Biol. 2:593-596, 1992), Vaswani and Hamilton (Ann. Allergy, Asthma and Immunol., 1:105-115 1998); Harris (Biochem. Soc. Transactions, 23:1035-1038, 1995); Hurle and Gross (Curr. Op. Biotech., 5:428-433, 1994), and specific prior art relating to humanization of V_{H}Hs such as e.g. Vincke et al. (2009, J. Biol. Chem. 284:3273-3284). Again, it should be noted that such humanized single variable domains of the invention can be obtained in any suitable manner known per se and thus are not strictly limited to polypeptides that have been obtained using a polypeptide that comprises a naturally occurring single variable domain as a starting material.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

As an alternative to humanization, human antibodies can be generated. By “human antibody” is meant an antibody containing entirely human light and heavy chains as well as constant regions, produced by any of the known standard methods. For example, transgenic animals (e.g., mice) are available that are capable, upon immunization, of producing a full repertoire of human antibodies in the absence of endogenous immunoglobulin production. For example, it has been described that the homozygous deletion of the antibody heavy-chain joining region PH gene in chimeric and germ-line mutant mice results in the complete inhibition of endogenous antibody production. Transfer of the human germ-line immunoglobulin gene array in such germ line mutant mice will result in the production of human antibodies after immunization. See, e.g., Jakobovits et al., Proc. Nat. Acad. Sci. USA, 90:255 1 (1993); Jakobovits et al., Nature, 362:255-258 (1993). Alternatively, phage display technology (McCafferty et al., Nature 348:552-553 (1990)) can be used to produce human antibodies and antibody fragments in vitro, from immunoglobulin variable (V) domain gene repertoires from donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B cell. Phage display can be performed in a variety of formats; for their review see, e.g., Johnson, Kevin S. and Chiswell, David J., Current Opinion in Structural Biology 3:564-57 1 (1993). Human antibodies may also be generated by in vitro activated B cells or SCID mice with its immune system reconstituted with human cells. Once a human antibody is obtained, its coding DNA sequences can be isolated, cloned and introduced into an appropriate expression system i.e. a cell line, preferably from a mammal, which subsequently express and liberate it into a culture media from which the antibody can be isolated.

The term “tumor associated antigen” (TAA) as used herein means any antigen including but not limited to a protein, glycoprotein, ganglioside, carbohydrate, lipid that is associated with cancer. Such antigen can be expressed on malignant cells or in the tumor microenvironment such as on tumor-associated blood vessels, extracellular matrix, mesenchymal stroma, or immune infiltrates. Expressly included in the term TAA are homologues of a wild-type TAA that differs therefrom as a result of tumor-specific mutations (which can be patient-specific or shared) and that result in altered amino acid sequences, i.e. so-called neoantigens.

A “nucleic acid construct” or “nucleic acid vector” is herein understood to mean a man-made nucleic acid molecule resulting from the use of recombinant DNA technology. The term “nucleic acid construct” therefore does not include naturally occurring nucleic acid molecules although a nucleic acid construct may comprise (parts of) naturally occurring nucleic acid molecules. The terms “expression vector” or expression construct” refer to nucleic acid molecules that are capable of effecting expression of a nucleotide sequence or gene in host cells or host organisms compatible with such expression vectors or constructs. These expression vectors typically include regulatory sequence elements that are operably linked to the nucleotide sequence to be expressed to effect its expression. Such regulatory elements usually at least include suitable transcription regulatory sequences and optionally, 3′ transcription termination signals. Additional elements necessary or helpful in effecting expression may also be present, such as expression enhancer elements. The expression vector will be introduced into a suitable host cell and be able to effect expression of the coding sequence in an in vitro cell culture of the host cell. The expression vector will be suitable for replication in the host cell or organism of the invention whereas an expression construct will usually integrate in the host cell's genome for it to be maintained. Techniques for the introduction of nucleic acid into cells are well established in the art and any suitable technique may be employed, in accordance with the particular circumstances. For eukaryotic cells, suitable techniques may include calcium phosphate transfection, DEAE-Dextran, electroporation, liposome-mediated transfection and transduction using retrovirus or other virus, e.g. adenovirus, AAV, lentivirus or vaccinia. For microbial, e.g. bacterial, cells, suitable techniques may include calcium chloride transformation, electroporation and transfection using bacteriophage. The introduced nucleic acid may be on an extra-chromosomal vector within the cell or the nucleic acid may be integrated into the genome of the host cell. Integration may be promoted by inclusion of sequences within the nucleic acid or vector which promote recombination with the genome, in accordance with standard techniques. The introduction may be followed by expression of the nucleic acid to produce the encoded fusion protein. In some embodiments, host cells (which may include cells actually transformed although more likely the cells will be descendants of the transformed cells) may be cultured in vitro under conditions for expression of the nucleic acid, so that the encoded fusion protein polypeptide is produced, when an inducible promoter is used, expression may require the activation of the inducible promoter.

As used herein, the term “promoter” or “transcription regulatory sequence” refers to a nucleic acid fragment that functions to control the transcription of one or more coding sequences, and is located upstream with respect to the direction of transcription of the transcription initiation site of the coding sequence, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active in most tissues under most physiological and developmental conditions. An “inducible” promoter is a promoter that is physiologically or developmentally regulated, e.g. by the application of a chemical inducer.

The term “selectable marker” is a term familiar to one of ordinary skill in the art and is used herein to describe any genetic entity which, when expressed, can be used to select for a cell or cells containing the selectable marker. The term “reporter” may be used interchangeably with marker, although it is mainly used to refer to visible markers, such as green fluorescent protein (GFP). Selectable markers may be dominant or recessive or bidirectional.

As used herein, the term “operably linked” refers to a linkage of polynucleotide elements in a functional relationship. A nucleic acid is “operably linked” when it is placed into a functional relationship with another nucleic acid sequence. For instance, a transcription regulatory sequence is operably linked to a coding sequence if it affects the transcription of the coding sequence. Operably linked means that the DNA sequences being linked are typically contiguous and, where necessary to join two protein encoding regions, contiguous and in reading frame.

The terms “protein” or “polypeptide” are used interchangeably and refer to molecules consisting of a chain of amino acids, without reference to a specific mode of action, size, 3-dimensional structure or origin.

The term “signal peptide” (sometimes referred to as signal sequence) is a short peptide (usually 16-30 amino acids long) present at the N-terminus of the majority of newly synthesized proteins that are destined towards the secretory pathway. At the end of the signal peptide there is usually a stretch of amino acids that is recognized and cleaved by signal peptidase either during or after completion of translocation (from the cytosol into the secretory pathway, i.e. ER) to generate a free signal peptide and a mature protein. Signal peptides are extremely heterogeneous, and many prokaryotic and eukaryotic signal peptides are functionally interchangeable even between different species however the efficiency of protein secretion may depend on the signal peptide. Suitable signal peptides are generally known in the art e.g. from Käll et al. (2004 J. Mol. Biol. 338:1027-1036) and von Heijne (1985, J Mol Biol. 184 (1): 99-105).

The term “gene” means a DNA fragment comprising a region (transcribed region), which is transcribed into an RNA molecule (e.g. an mRNA) in a cell, operably linked to suitable regulatory regions (e.g. a promoter). A gene will usually comprise several operably linked fragments, such as a promoter, a 5′ leader sequence, a coding region and a 3′ non-translated sequence (3′ end) comprising a polyadenylation site. “Expression of a gene” refers to the process wherein a DNA region which is operably linked to appropriate regulatory regions, particularly a promoter, is transcribed into an RNA, which is biologically active, i.e. which is capable of being translated into a biologically active protein or peptide.

The term “homologous” when used to indicate the relation between a given (recombinant) nucleic acid or polypeptide molecule and a given host organism or host cell, is understood to mean that in nature the nucleic acid or polypeptide molecule is produced by a host cell or organisms of the same species, preferably of the same variety or strain. If homologous to a host cell, a nucleic acid sequence encoding a polypeptide will typically (but not necessarily) be operably linked to another (heterologous) promoter sequence and, if applicable, another (heterologous) secretory signal sequence and/or terminator sequence than in its natural environment. It is understood that the regulatory sequences, signal sequences, terminator sequences, etc. may also be homologous to the host cell. When used to indicate the relatedness of two nucleic acid sequences the term “homologous” means that one single-stranded nucleic acid sequence may hybridize to a complementary single-stranded nucleic acid sequence. The degree of hybridization may depend on a number of factors including the amount of identity between the sequences and the hybridization conditions such as temperature and salt concentration as discussed later.

The term “heterologous” when used with respect to a nucleic acid (DNA or RNA) or protein refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins may also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

### DETAILED DESCRIPTION OF THE INVENTION

The present invention arises in part from the observation that multispecific antigen binding proteins which comprise an NK cell-activating cytokines that triggers at least one of the NK cell's interleukin 21 receptor and 4-1BB, and which comprise a region that has affinity for a surface antigen expressed on NK cells, such as an Fc region, are capable of inducing hyper-functionality in NK cells, which includes their proliferation, resistance to the tumor microenvironment, their enhanced capability to mediate lysis of the target cell and the prolongation of these capabilities (FIG. 1). Such capabilities can be even further enhanced if the multispecific antigen binding protein comprises at least one antigen-binding region that specifically binds an NK cell activating receptor.

### a Multispecific Antigen Binding Protein

In a first aspect, the invention pertains to a multispecific antigen binding protein. In one embodiment, the multispecific antigen binding protein comprises a) an NK cell-activating cytokine; and, ii) a 4-1BB agonist; and, b) a region that has affinity for a surface antigen expressed on natural killer (NK) cells. The NK cell-activating cytokine preferably is at least one of i) an interleukin 21 receptor (IL21R) agonist; and, ii) a 4-1BB agonist. The region with affinity for NK cell surface antigen allows the multispecific antigen binding protein to bind to an NK cell, after which the NK cell-activating cytokine(s) can induce hyper-functionality phenotype in the NK cell. In one embodiment, the multispecific antigen binding protein further comprises c) at least one antigen-binding region that specifically binds an NK cell activating receptor.

Thus, in one embodiment, the multispecific antigen binding protein comprises: a) an NK cell-activating cytokine that is at least one of: i) an interleukin 21 receptor (IL21R) agonist; and, ii) a 4-1BB agonist; b) a region that has affinity for a surface antigen expressed on NK cells; and, c) at least one antigen-binding region that specifically binds an NK cell activating receptor; an NK cell activating receptor.

### NK Cell-Activating Cytokines

A multispecific antigen binding protein as described herein, thus comprises at least one NK cell-activating cytokine. In one embodiment, NK cell-activating cytokine that is at least one of: i) an interleukin 21 receptor (IL21R) agonist; and, ii) a 4-1BB agonist. In one embodiment, the multispecific antigen binding protein at least comprises an IL21R agonist. In one embodiment, the multispecific antigen binding protein at least comprises a 4-1BB agonist. And, in one embodiment, the multispecific antigen binding protein at least comprises both an IL21R agonist and a 4-1BB agonist. In further embodiments, the multispecific antigen binding protein as described herein can comprises further NK cell-activating cytokines, in addition to at least one of an IL21R agonist and a 4-1BB agonist. Such further NK cell-activating cytokines can be selected from the group consisting of an IL15 receptor agonist, an IL2 receptor agonist, an IL12 receptor agonist and an IL18 receptor agonist, as further detailed below.

Thus, in one embodiment, the multispecific antigen binding protein as described herein at least comprises an interleukin 21 receptor (IL21R) agonist.

Interleukin 21 (IL21) is a protein that in humans is encoded by the IL21 gene (Entrez Gene ID: 59067). IL21 is a cytokine that has potent regulatory effects on cells of the immune system, including natural killer (NK) cells and which induces cell division/proliferation in its target cells. Amino acid sequences for human IL21 precursor (including its signal sequence) are described in NCBI accession numbers NP_001193935 and NP_068575, the disclosures of which are incorporated herein by reference. IL21 (mature/processed) comprises amino acids 30-153 of NP_001193935 or amino acids 30-162 of NP_068575 (i.e. SEQ ID NO: 38). IL21 exerts its effects on target cells through the IL-21 receptor (IL21R) is expressed on the surface of T, B and NK cells. IL21R is similar in structure to the receptors for other type I cytokines like IL-2R or IL-15 and requires dimerization with the common gamma chain (γc) in order to bind IL-21. IL21R is encoded in humans by the IL21R gene (Entrez Gene ID: 50615). Amino acid sequences for human IL21R are described in NCBI accession numbers NP_068570, NP_851564 and NP_851565, the disclosures of which are incorporated herein by reference.

As used herein, an “IL21R agonist” is an agent that has “agonist” activity at the IL21 receptor, which means that the agent that can cause or increase “IL21R signaling”. “IL21R signaling” refers to an ability of IL21R, e.g. when expressed on the surface of T, B and NK cells and triggered by its natural ligand IL21, to activate or transduce an intracellular signaling pathway. The “natural ligand IL21” is herein understood as a human wild type IL21 comprising or consisting of an amino acid sequence as indicated above. When bound to IL-21, the IL-21 receptor acts through the Jak/STAT pathway, utilizing Jak1 and Jak3 and a STAT3 homodimer to activate its target genes. IL21R agonist activity, i.e. changes in IL21R signaling activity, can be measured, for example, by assays designed to measure changes in the IL21R signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of IL21R-sensitive promoters and enhancers, or indirectly by a downstream effect mediated by IL21R (e.g. activation of specific cytolytic machinery in NK cells). A suitable cell-based assay for biological activity of an IL21R agonist, is e.g. described in Maurer et al. (mAbs. 2012; 4 (1): 69-83.), wherein a murine pre-B-cell line is transfected with both the human IL21R and a STAT-responsive luciferase reporter gene. IL21R agonist activity can be determined using this cell line by measuring the level of STAT3 phosphorylation using anti-pSTAT3 antibody-conjugated beads and/or by detecting luciferase luminescence, upon contacting the cell line with an IL21R agonist. The natural ligand IL21 can serve as a positive control in an assay for IL21R agonist activity and can also be used as a reference for the amount of IL21R agonist activity of a given non-natural IL21R agonist, such as a multispecific antigen binding protein as described herein comprising an IL21R agonist.

In one embodiment, a multispecific antigen binding protein as described herein comprises an IL21R agonist that has reduced IL21R agonist activity as compared to human wild type IL21. In one embodiment, the IL21R agonist has an IL21R agonist activity that is a factor 2, 5, 10, 20, 50, 100, 200, 500 or 1000 less than that of human wild type IL21.

In one embodiment, a multispecific antigen binding protein as described herein comprises an IL21R agonist that has enhanced IL21R agonist activity as compared to human wild type IL21. In one embodiment, the IL21R agonist has an IL21R agonist activity that is a factor 2, 5, 10, 20, 50, 100, 200, 500 or 1000 higher than that of human wild type IL21.

In one embodiment, a multispecific antigen binding protein as described herein comprises an IL21R agonist that is an IL21 polypeptide comprising an amino acid sequence with at least 50, 55, 60, 65, 70, 75, 80, 85, 90, 95, 96, 97, 98, 99, or 100% sequence identity to SEQ ID NO: 38, and preferably having an IL21R agonist activity as defined above, and/or preferably having an affinity for the IL21R as defined below.

In one embodiment, a multispecific antigen binding protein as described herein comprises an IL21R agonist of which the affinity for the IL21R is reduced or enhanced as compared to human wild type IL21. The affinity of an IL21R agonist of the affinity for the IL21R can be assayed using methods generally known in the art, such as surface plasmon resonance.

In one embodiment, a multispecific antigen binding protein as described herein comprises an IL21R agonist that has reduced affinity for IL21R as compared to human wild type IL21. In one embodiment, the affinity of the IL21R agonist for IL21R is a factor 2, 5, 10, 20, 50, 100, 200, 500, 1000, 10000, or 100000 less than that of human wild type IL21.

In one embodiment, a multispecific antigen binding protein as described herein comprises an IL21R agonist that has enhanced affinity for IL21R as compared to human wild type IL21. In one embodiment, the affinity of the IL21R agonist for IL21R is a factor 2, 5, 10, 20, 50, 100, 200, 500 or 1000 higher than that of human wild type IL21.

In one embodiment, a multispecific antigen binding protein as described herein comprises an IL21R agonist that is IL21 or a fragment thereof that has IL21R agonist activity. Preferably, the IL21R agonist is human IL21 or a fragment thereof that has IL21R agonist activity. In one embodiment, the IL21R agonist is an IL21 mutein with reduced affinity for IL21R as compared to human wild type IL21. IL21 muteins with reduced affinity for IL21R as compared to human wild type IL21 are described in Shen et al. (Front Immunol. 2020; 11:832). Thus, in one embodiment, the IL21R agonist is an IL21 mutein with a mutation (i.e. amino acid substitution, deletion or insertion) of one or more amino acids selected from the group consisting of 116, 166, 18, K72, K73, K75, K77, L13, P78, Q12, Q19, R5, R65, R76, R9, S70, S80, V69 and Y23 (amino acid positions referring to position in SEQ ID NO: 38 or a corresponding position in an IL-21 allelic variant). Preferably, the IL21R agonist is an IL21 mutein comprising one or more amino acid substitutions selected from the group consisting of I8A, K72D, K73A, K75D, K77D, L13D, P78D, Q12A, Q19D, R5A, R65D, R76A, R9A, S70E, S80G, V69D, Y23D, I16E, I66G, I8D, K72G, K73D, K75G, K77G, P79D, Q12D, R5D, R65G, R76D, R9D, S70G, S80P, V69G, I66P, I8E, K72P, K73E, K75P, K77P, Q12E, R5E, R65P, R76E, R9E, S70P, V69P, I8G, K73G, Q12N, R5G, R76G, R9G, S70Y, I8N, K73H, Q12S, R5H, R76H, R9H, I8S, K73I, Q12T, R51, R76I, R9I, K73N, Q12V, R5K, R76K, R9K, K73P, R5L, R76L, R9L, K73Q, R5M, R76M, R9M, K73S, R5N, R76N, R9N, K73V, R5Q, R76P, R9Q, R5S, R76Q, R9S, R5T, R76S, R9T, R5V, R76T, R9V, R5Y, R76V, R9Y and R76Y.

In one embodiment, the IL21R agonist is an IL21 mutein that has an affinity for human IL21R-Fc in Table 2 of Shen et al. (2020; supra) between 0.028 and 0.099 nM. In one embodiment, the IL21R agonist is an IL21 mutein that has an affinity for human IL21R-Fc in Table 2 of Shen et al. (2020; supra) between 0.10 and 0.29 nM. In one embodiment, the IL21R agonist is an IL21 mutein that has an affinity for human IL21R-Fc in Table 2 of Shen et al. (2020; supra) between 0.30 and 0.99 nM. In one embodiment, the IL21R agonist is an IL21 mutein that has an affinity for human IL21R-Fc in Table 2 of Shen et al. (2020; supra) between 1.0 and 2.9 nM. In one embodiment, the IL21R agonist is an IL21 mutein that has an affinity for human IL21R-Fc in Table 2 of Shen et al. (2020; supra)>2.9 nM.

In one embodiment, a multispecific antigen binding protein as described herein comprises an IL21R agonist that is an antigen-binding region that specifically binds IL21R and that has IL21R agonist activity. The antigen-binding region can be an antigen-binding region as described herein above.

In one embodiment, a multispecific antigen binding protein as described herein comprises more than one IL21R agonist as described above. Thus, in one embodiment, the multispecific antigen binding protein has an IL21R agonist-valency that is higher than one. The IL21R agonist-valency of a multispecific antigen binding protein can for example be 2, 3, 4, 5, 6, 7, 8, 9, 10, 11, 12, 13, 14, 15 or more.

In one embodiment, the multispecific antigen binding protein as described herein at least comprises a 4-1BB agonist.

4-1BB is a member of the tumor necrosis factor receptor family. Its alternative names are tumor necrosis factor receptor superfamily member 9 (TNFRSF9), CD137 and induced by lymphocyte activation (ILA). 4-1BB is encoded by the TNFRSF9 gene (Entrez Gene ID: 3604). An amino acid sequence for human 4-1BB is described in NCBI accession numbers NP_001552, the disclosure of which is incorporated herein by reference. 4-1BB is known as a co-stimulatory immune checkpoint molecule. 4-1BB is expressed by activated T cells of both the CD4+ and CD8+ lineages, as well as on activated NK cells. NK cells with increased 4-1BB expression are known to be highly active against target cells (e.g. tumor cells) expressing 4-1BB ligand. 4-1BB ligand (4-1BBL), also known as TNFSF9 or CD137L, is a protein that in humans is encoded by the TNFSF9 gene (Entrez Gene ID: 8744). An amino acid sequence for human 4-1BBL is described in NCBI accession numbers NP_003802, the disclosure of which is incorporated herein by reference. The 4-1BB/4-1BBL complex consists of three monomeric 4-1BBs bound to a trimeric 4-1BBL. Each 4-1BB monomer binds to two 4-1BBLs via cysteine-rich domains (CRDs). The interaction between 4-1BB and the second 4-1BBL is required to stabilize their interactions.

As used herein, an “4-1BB agonist” is an agent that has “agonist” activity at the 4-1BB, which means that the agent that can cause or increase “4-1BB signaling”. “4-1BB signaling” refers to an ability of 4-1BB, e.g. when expressed on the surface of T, B and NK cells and triggered by its natural ligand 4-1BBL, to activate or transduce an intracellular signaling pathway. The “natural 4-1BB ligand” is herein understood as the extracellular domain (ECD) of a human wild type 4-1BBL comprising or consisting of an amino acid sequence from position 71 to 254 of the amino acid sequence of human 4-1BBL (i.e. SEQ ID NO: 37). A 4-1BBL extracellular domain (ECD) is herein thus understood as a polypeptide comprising or consisting of an amino acid sequence from positions 71 to 254 of human 4-1BBL, or a fragment thereof having 4-1BB agonist activity.

4-1BB agonist activity, i.e. changes in 4-1BB signaling activity, can be measured, for example, by assays designed to measure changes in the 4-1BB signaling pathways, e.g. by monitoring phosphorylation of signal transduction components, assays to measure the association of certain signal transduction components with other proteins or intracellular structures, or in the biochemical activity of components such as kinases, or indirectly by a downstream effect mediated by 4-1BB (e.g. production of specific cytokines). A suitable cell-based assay for in vitro biological activity of a 4-1BB agonist, is e.g. described in Zhang et al. (Clin Cancer Res, 2007; 13 (9): 2758-2767), using measurement of IL-2 production from splenocytes aseptically removed from BALB/c mice in microtiter plates precoated with an anti-CD3 monoclonal antibody (145-11C clone). Other suitable cell-based assays for in vitro biological activity of a 4-1BB agonist, are described in WO2016/075278, Example 6 (see e.g. Example 6.1). The natural 4-1BB ligand, a 4-1BBL ECD trimer as described by Fellermeier et al. (Oncoimmunol. 2016, 5 (11): e1238540), e.g. a 4-1BBL ECD trimer comprising the amino acid sequence of SEQ ID NO: 36, or an anti-CD137 agonist antibody (such the antibody 2A, Epstein et al., Tumor necrosis imaging and treatment of solid tumors. In: V. P. Torchilin, editor. Handbook of targeted delivery of imaging agents, Vol. 16. Boca Raton: CRCPress; 1995. p. 259.) can serve as a positive control in an assay for 4-1BB agonist activity and can also be used as a reference for the amount of 4-1BB agonist activity of a given non-natural 4-1BB agonist, such as a multispecific antigen binding protein as described herein comprising a 4-1BB agonist.

In one embodiment, a multispecific antigen binding protein as described herein comprises a 4-1BB agonist that has reduced 4-1BB agonist activity as compared to human wild type 4-1BBL or the anti-4-1BB agonist antibody 2A. In one embodiment, the 4-1BB agonist has a 4-1BB agonist activity that is a factor 2, 5, 10, 20, 50, 100, 200, 500 or 1000 less than that of the ECD of human wild type 4-1BBL or the anti-4-1BB agonist antibody 2A.

In one embodiment, a multispecific antigen binding protein as described herein comprises a 4-1BB agonist that has enhanced 4-1BB agonist activity as compared to human wild type 4-1BBL. In one embodiment, the 4-1BB agonist has a 4-1BB agonist activity that is a factor 2, 5, 10, 20, 50, 100, 200, 500 or 1000 higher than that of the ECD of human wild type 4-1BBL or the anti-4-1BB agonist antibody 2A.

In one embodiment, a multispecific antigen binding protein as described herein comprises a 4-1BB agonist comprising at least one 4-1BBL ECD comprising an amino acid sequence with at least 50, 55, 60, 65, 70, 75, 80, 85, 90, 95, 96, 97, 98, 99, or 100% sequence identity to SEQ ID NO: 37, and preferably having an 4-1BB agonist activity as defined above, and/or preferably having an affinity for 4-1BB as defined below.

In one embodiment, a multispecific antigen binding protein as described herein comprises a 4-1BB agonist of which the affinity for the 4-1BB is reduced or enhanced as compared to the ECD of human wild type 4-1BBL. The affinity of a 4-1BB agonist of the affinity for the 4-1BB can be assayed using methods generally known in the art, such as surface plasmon resonance.

In one embodiment, a multispecific antigen binding protein as described herein comprises a 4-1BB agonist that has reduced affinity for 4-1BB as compared to human wild type 4-1BBL. In one embodiment, the affinity of the 4-1BB agonist for 4-1BB is a factor 2, 5, 10, 20, 50, 100, 200, 500, 1000 or 10000 less than that of the ECD of human wild type 4-1BBL or the anti-4-1BB agonist antibody 2A.

In one embodiment, a multispecific antigen binding protein as described herein comprises a 4-1BB agonist that has enhanced affinity for 4-1BB as compared to human wild type 4-1BBL. In one embodiment, the affinity of the 4-1BB agonist for 4-1BB is a factor 2, 5, 10, 20, 50, 100, 200, 500 or 1000 higher than that of the ECD of human wild type 4-1BBL or the anti-4-1BB agonist antibody 2A.

In one embodiment, a multispecific antigen binding protein as described herein comprises a 4-1BB agonist that comprises or consists of the ECD of 4-1BBL or a fragment thereof that has 4-1BB agonist activity. Preferably, the 4-1BB agonist is human 4-1BBL or a fragment thereof that has 4-1BB agonist activity. In one embodiment, the 4-1BB agonist is a mutein of the ECD of 4-1BBL with reduced affinity for 4-1BB as compared to the ECD of human wild type 4-1BBL or the anti-4-1BB agonist antibody 2A.

In one embodiment, a multispecific antigen binding protein as described herein comprises a 4-1BB agonist that comprises or consists of a fusion protein comprising three 4-1BBL ECD monomers fused together in a single polypeptide chain, as e.g. described in Fellermeier et al. (2016, supra). In one embodiment, the three 4-1BBL ECD monomers are connected by polypeptide linkers. In one embodiment, the three 4-1BBL ECD monomers are connected by polypeptide linkers selected from the group consisting of (GGGGS)₄, GGGSGGG, GGSGGGGSGG and G, of which (GGGGS)₄ is preferred. Other suitable flexible polypeptide linker(s) are described herein below. In one embodiment, a multispecific antigen binding protein as described herein comprises a 4-1BB agonist that comprises or consists of a fusion protein comprising three 4-1BBL ECD monomers fused together in a single polypeptide chain, e.g. comprising the amino acid sequence of SEQ ID NO: 36.

In one embodiment, a multispecific antigen binding protein as described herein comprises a 4-1BB agonist that comprises three 4-1BBL ECD monomers that are present in more than one polypeptide chain of the multispecific antigen binding protein. For example, two 4-1BBL ECD monomers can be fused together in a single polypeptide chain, optionally connected together through polypeptide linker as described above, that is part of a first polypeptide chain of the multispecific antigen binding protein and the third 4-1BBL ECD monomer is part of a second polypeptide chain of the multispecific antigen binding protein as e.g. described in WO2016/075278. The first and second polypeptide chains of the multispecific antigen binding protein can be the chains comprising the heavy and light chains, respectively, or vice versa, whereby preferably the 4-1BBL ECDs are fused to the N-termini of the variable domains. Alternatively, the first and second polypeptide chains of the multispecific antigen binding protein can be the two chains comprising the two heavy chains, whereby preferably the 4-1BBL ECDs are fused to the C-termini of the constant domains.

In one embodiment, a multispecific antigen binding protein as described herein comprises a 4-1BB agonist that comprises a antigen-binding region that specifically binds 4-1BB and that has 4-1BB agonistic activity. Antibodies against 4-1BB are e.g. described in WO2005035584, WO2006088464, US2006188439.

In one embodiment, a multispecific antigen binding protein as described herein comprises more than one 4-1BB agonist as described above. Thus, in one embodiment, the multispecific antigen binding protein has a 4-1BB agonist-valency that is higher than one. The 4-1BB agonist-valency of a multispecific antigen binding protein can for example be 2, 3, 4, 5, 6, 7, 8, 9, 10, 11, 12, 13, 14, 15 or more.

In a preferred embodiment, a multispecific antigen binding protein as described herein comprises both at least one IL21R agonist as herein described above and at least one 4-1BB agonist as herein described above.

In further embodiments, the multispecific antigen binding protein as described herein can comprise, in addition to at least one of an IL21R agonist and a 4-1BB agonist, a further NK cell-activating cytokines selected from the group consisting of an IL15 receptor agonist, a type I interferon (IFN-1) agonist, an IL2 receptor agonist, an IL12 receptor agonist and an IL18 receptor agonist. In one embodiment, the IL15 receptor agonist is an IL15 polypeptide or an agonistic antigen-binding region that specifically binds the IL15 receptor. In one embodiment, the IL2 receptor agonist is an IL2 polypeptide or an agonistic antigen-binding region that specifically binds the IL2 receptor. In one embodiment, the IL12 receptor agonist is an IL12 polypeptide or an agonistic antigen-binding region that specifically binds the IL12 receptor. In one embodiment, the IL18 receptor agonist is an IL18 polypeptide or an agonistic antigen-binding region that specifically binds the IL18 receptor.

a Region with Affinity for a Surface Antigen Expressed on NK Cells

In one embodiment, a multispecific antigen binding protein as described herein, can further comprise a region that has affinity for a surface antigen expressed on NK cells. Thus, the presence of such a region with affinity for a surface antigen expressed on NK cells in the multispecific antigen binding protein is optional.

In one embodiment of the multispecific antigen binding protein, the region that has affinity for a surface antigen expressed on NK cells cells comprises or consists of an immunoglobulin Fc region, or at least a portion thereof that binds the type III Fcγ receptor (FcγRIIIa) as expressed on (human) NK cells, also referred to herein as CD16A. In one embodiment, the immunoglobulin Fc region at least comprises at least one of a CH2 and CH3 domain. In one embodiment, the immunoglobulin Fc region at least comprises at least one of a CH2 and CH3 domain and a hinge region. In one embodiment, the immunoglobulin Fc region comprises or consists of a hinge region and a CH2 and CH3 domain. In one embodiment, the immunoglobulin Fc region is a dimeric Fc region or at least a portion thereof that binds CD16A.

In one embodiment, an Fc region or portion thereof that binds CD16A be a wild-type region or portion thereof.

In one embodiment, an Fc region or portion thereof that binds CD16A can be modified to enhance or reduce its binding affinity to CD16A. Within the Fc region, CD16A binding is mediated by the hinge region and the CH2 domain. For example, within human IgG1, the interaction with CD16 is primarily focused on amino acid residues D265-E269, N297-T299, A327-1332, L 234-S239, and carbohydrate residue N-acetyl-D-glucosamine in the CH2 domain (see, Sondermann et al., 2000 Nature, 406 (6793): 267-273). Based on the known domains, mutations can be selected to enhance or reduce the binding affinity to CD16A, such as by using phage-displayed libraries or yeast surface-displayed cDNA libraries or can be designed based on the known three-dimensional structure of the interaction.

Thus, in one embodiment, where a multispecific antigen binding protein is intended to have increased affinity for CD16A, an Fc region or portion thereof that binds CD16A, can comprise a modification to increase affinity for CD16A. Thus, an Fc region or portion thereof that binds CD16A, can comprise one or more amino acid modifications (e.g. amino acid substitutions, deletions, insertions) which increase binding to (human) CD16A and optionally another receptor such as FcRn. Typical modifications include modified human IgG1-derived constant regions comprising at least one amino acid modification (e.g. substitution, deletions, insertions), and/or altered types of glycosylation, e.g., hypofucosylation. A modification can, for example, increase binding of an Fc region to FcyRIIIa (CD16A) on NK cells. Examples of modifications are provided in U.S. Pat. No. 10,577,419, the disclosure of which is incorporated herein by reference. Specific mutations (in IgG1 Fc regions) which enhance FcyRIIIa (CD16A) binding, include E333A, S239D/I332E and S239D/A330L/I332E.

In one embodiment, the multispecific antigen binding protein comprises an Fc region or portion thereof that binds CD16A comprising at least one amino acid modification (for example, 1, 2, 3, 4, 5, 6, 7, 8, 9, or more amino acid modifications) relative to a wild-type Fc region, such that the molecule has enhanced binding affinity for (human) CD16A relative to a molecule comprising a wild-type Fc region, optionally wherein the variant Fc region comprises a substitution at any one or more of positions 239, 298, 330, 332, 333 and/or 334 (e.g. S239D, S298A, A330L, I332E, E333A and/or K334A substitutions), optionally wherein the variant Fc region comprises a substitution at residues S239 and I332, e.g. a S239D and I332E substitution (Kabat EU numbering).

In one embodiment, the multispecific antigen binding protein comprises an Fc region or portion thereof that binds CD16A comprising altered glycosylation patterns that increase binding affinity for (human) CD16A. Such carbohydrate modifications can be accomplished by, for example, by expressing a nucleic acid encoding the multispecific protein in a host cell with altered glycosylation machinery. Cells with altered glycosylation machinery are known in the art and can be used as host cells in which to express recombinant antibodies to thereby produce an antibody with altered glycosylation. See, for example. Shields, R. L. et al. (2002) J. Biol. Chem. 277:26733-26740; Umana et al. (1999) Nat. Biotech. 17:176-1, as well as, European Patent No: EP 1,176,195; WO 06/133148; WO 03/035835; WO 99/54342, each of which is incorporated herein by reference in its entirety. In one embodiment, the multispecific antigen binding protein comprises one or more hypofucosylated constant regions. Such a multispecific antigen binding protein can comprise an amino acid alteration or cannot comprise an amino acid alteration and/or may be expressed or synthesized or treated under conditions that result in hypofucosylation. In one embodiment, in a composition comprising the multispecific antigen binding protein described herein, at least 20, 30, 40, 50, 60, 75, 85, 90, 95% or substantially all of the multispecific antigen binding protein have a constant region comprising a core carbohydrate structure (e.g. complex, hybrid and high mannose structures) which lacks fucose. In one embodiment, provided is a multispecific antigen binding protein which is free of N-linked glycans comprising a core carbohydrate structure having fucose. The core carbohydrate will preferably be a sugar chain at Asn297.

In one embodiment, the multispecific antigen binding protein comprising an Fc region or portion thereof that binds CD16A that is modified to have increased binding affinity for CD16A, has a binding affinity for human CD16A that is at least 1, 2 or 3 log greater than that of a conventional or wild-type human IgG1 antibody, e.g., as assessed by surface plasmon resonance.

In another embodiment, where a multispecific antigen binding protein is intended to have reduced affinity for CD16A, a CH2 and/or CH3 domain, an Fc region or portion thereof that binds CD16A, can comprise a modification to decrease affinity for CD16A. For example, CH2 mutations in a dimeric Fc region protein at reside N297 (Kabat numbering) can eliminate CD16A binding. Other modification in the Fc region that reduce or eliminate binding to CD16A include the L234A/L235A, also known as “LALA” modifications, as exemplified herein. Modification of the Fc region that reduce or eliminate its binding to CD16A can be useful in a multispecific antigen binding protein to reduce or avoid NK cell fratricide. The lack of NK cell fratricide can be an advantageous feature for the multispecific antigen binding protein described herein. NK cell cross-linking with NK cells or other immune cells is expected to reduce therapeutic efficacy of NK cell-engagement. Most importantly, cross-linking of a NK cell with one or more NK cells or other immune cells through bivalent or multivalent interactions with FcRy or in combination with a second immune cell antigen (e.g. NKp46, NKG2D, NKp30 or SLAMF7) can cause immune cell activation. This might lead to induction of target cell-driven fratricide or immune cell killing (e.g. NK-NK cell lysis), ultimately resulting in efficient NK cell depletion in vivo, as previously described for a CD16-directed murine IgG antibody (3G8), the CD38-directed antibody daratumumab and other approaches (Choi et al 2008 Immunology 124 (2) 215-22; DOI: 10.111 l/j.I365-2567.2007.02757.x; Yoshida 2010 Front. Microbiol 1:128 DOI: 10.3389/fmicb.2010.00128; Wang et al 2018 Clin Cancer Res, 24 (16): 4006-4017; DOI: 10.1158/1078-0432.CCR-17-3117; His et al 2008; Nakamura 2013 PNAS; 110 (23) 9421-9426; DOI: 10.1073/pnas.1300140110; Breman et al 2018 Front Immunol, 12 (9) 2940; DOI: 10.3389/fimmu.2018.02940).

The person of skill in the art will appreciate that other configurations for modification of Fc regions can be implemented. For example, substitutions into human IgG1 or IgG2 residues at positions 233-236 and IgG4 residues at positions 327, 330 and 331 were shown to greatly reduce binding to Fcy receptors and thus ADCC and CDC. Furthermore, Idusogie et al. (2000) J. Immunol. 164 (8): 4178-84 demonstrated that alanine substitution at different positions, including K322, significantly reduced complement activation.

In one embodiment, the multispecific antigen binding protein comprising an Fc region or portion thereof that binds CD16A that is modified to have reduced binding affinity for CD16A, has a binding affinity for human CD16A that is at least 1, 2 or 3 log less than that of a conventional or wild-type human IgG1 antibody, e.g., as assessed by surface plasmon resonance.

In one embodiment, the multispecific antigen binding protein comprises an Fc region that has an amino acid sequence having at least 85, 90, 91, 92, 93, 94, 95, 96, 97, 98, 99 or 100% amino acid identity with an Fc region in at least one of SEQ ID NO.'s: 1, 3, 11-19 and 23, and preferably having one or more of the above structural and/or functional features.

Antigen-Binding Regions that Specifically Binds an NK Cell Activating Receptor

In one embodiment, a multispecific antigen binding protein described herein further comprises an antigen-binding region that specifically binds an NK cell activating receptor.

An antigen-binding region as used in a multispecific antigen binding protein described herein can be derived from any of a variety of immunoglobulin or non-immunoglobulin scaffolds, for example affibodies based on the Z-domain of staphylococcal protein A, engineered Kunitz domains, monobodies or adnectins based on the 10th extracellular domain of human fibronectin III, anticalins derived from lipocalins, DARPins (designed ankyrin repeat domains), Affilins, multimerized LDLR-A module, avimers or cysteine-rich knottin peptides. See, e.g., Gebauer and Skerra (2009) Current Opinion in Chemical Biology 13:245-255, the disclosure of which is incorporated herein by reference. In an alternative embodiment, the antigen-binding region that specifically binds an NK cell activating receptor is a ligand, e.g. a peptide ligand, that specifically binds, and preferably activates, an NK cell activating receptor.

In a preferred embodiment, an antigen-binding region as used in a multispecific antigen binding protein described herein comprises or consists of an immunoglobulin variable region. Such immunoglobulin variable regions can comprise or consist of variable domains that are commonly derived from antibodies (immunoglobulin chains), e.g. in the form of associated V_{L} and V_{H} domains found on two polypeptide chains, such as present in a Fab. Alternatively, immunoglobulin variable regions can comprise or consist of a single chain antigen binding domain such as a scFv, a V_{H} domain, a V_{L} domain, or an immunoglobulin single variable domain (ISVD) such as a dAb, a V-NAR domain or a V_{H}H domain. An immunoglobulin variable region to be used in a multispecific antigen binding protein described herein can be a human or humanized immunoglobulin variable region or an immunoglobulin single variable domain as herein defined above.

In one embodiment of a multispecific antigen binding protein described herein, the antigen-binding region that specifically binds an NK cell activating receptor is an agonistic antigen-binding region that activates the NK cell receptor. As used herein, an antigen-binding region that has “agonist” activity at an NK cell activating receptor is an agent that can cause or increase “signaling by the NK cell activating receptor”. “Signaling by the NK cell activating receptor” refers to an ability of an NK cell activating receptor to activate or transduce an intracellular signaling pathway. Changes in NK cell activating receptor-signaling activity can be measured, for example, by assays designed to measure changes in NK cell activating receptor-signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of NK cell activating receptor-sensitive promoters and enhancers, or indirectly by a downstream effect mediated by the NK cell activating receptor polypeptide (e.g. activation of specific cytolytic machinery in NK cells). Reporter genes can be naturally occurring genes (e.g. monitoring cytokine production) or they can be genes artificially introduced into a cell. Other genes can be placed under the control of such regulatory elements and thus serve to report the level of NK cell activating receptor-signaling activity.

In one embodiment, the antigen-binding region that specifically binds an NK cell activating receptor is an antigen-binding region derived from a of immunoglobulin or non-immunoglobulin scaffolds as defined above. Preferably, the antigen-binding region that specifically binds an NK cell activating receptor comprises or consists of at least one immunoglobulin variable region. More preferably, the antigen-binding region that specifically binds an NK cell activating receptor comprises or consists of a Fab that specifically binds an NK cell activating receptor or an immunoglobulin single variable domain (ISVD) that specifically binds an NK cell activating receptor. In one embodiment, the antigen-binding region that specifically binds an NK cell activating receptor is an antigen-binding region that binds the NK cell activating receptor with a K_{D} value of no more than 10⁻⁴ M, as may be determined as herein described above.

In one embodiment, the antigen-binding region that specifically binds an NK cell activating receptor comprises or consists of a human or humanized immunoglobulin variable region or immunoglobulin single variable domain as herein defined above.

In one embodiment, a multispecific antigen binding protein as described herein comprises two antigen-binding regions that specifically bind an NK cell activating receptor. In a multispecific antigen binding protein that comprises two antigen-binding regions that specifically bind an NK cell activating receptor, the two antigen-binding regions can bind one and the same NK cell activating receptor, or they can at least two different NK cell activating receptors. In one embodiment of a multispecific antigen binding protein that comprises two antigen-binding regions that specifically bind an NK cell activating receptor, the two antigen-binding regions are identical. Thus, as regards the two antigen-binding regions that specifically bind an NK cell activating receptor, a multispecific antigen binding protein as described herein can be a homodimeric or a heterodimeric antigen binding protein.

In one embodiment, a multispecific antigen binding protein as described herein comprises the antigen-binding region that specifically binds an NK cell activating receptor selected from the group consisting of: NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-BB, OX40, CRTAM, CD27, PSGL1, CD96, CD100, CEACAM1, CD59, PD-L1, Tim3 and NTB-A, of which NKp46, NKp30, NKG2D, CD16A, and SLAMF7 are preferred.

In one embodiment, a multispecific antigen binding protein as described herein comprises the antigen-binding region that specifically binds an NK cell activating receptor that is a natural cytotoxicity receptor (NCR). Natural cytotoxicity receptors are type 1 transmembrane proteins of the immunoglobulin superfamily, which upon stimulation mediate NK killing and release of IFNγ. They bind viral ligands such as hemagglutinins and hemagglutinin neuraminidases, some bacterial ligands and cellular ligands related to tumour growth such as PCNA. Natural Cytotoxicity Receptors include NKp46, NKp44, and NKp30. In one embodiment, a multispecific antigen binding protein as described herein comprises the antigen-binding region that specifically binds an NK cell activating receptor that is an NCR selected from the group consisting of NKp46, NKp44 and NKp30.

“NKp46” refers to a protein or polypeptide encoded by an Ncr1 gene or by a cDNA prepared from such a gene. NKp46 has also been designated as NCR1, CD335 (cluster of differentiation, NKP46, NK-p46, and LY94. Any naturally occurring isoform, allele, ortholog or variant is encompassed by the term NKp46 polypeptide (e.g., an NKp46 polypeptide 90%, 95%, 98% or 99% identical to SEQ ID NO: 50, or a contiguous sequence of at least 20, 30, 50, 100 or 200 amino acid residues thereof). The 304 amino acid residue sequence of human NKp46 (isoform a) is shown in in SEQ ID NO: 50, which corresponds to NCBI accession number NP_004820, the disclosure of which is incorporated herein by reference. The human NKp46 mRNA sequence is described in NCBI accession number NM_004829, the disclosure of which is incorporated herein by reference.

“NKp44” refers to a protein or polypeptide encoded by an Ncr2 gene or by a cDNA prepared from such a gene. NKp44 has also been designated as NCR2, CD336 (cluster of differentiation 336), NKP44, NK-p44, LY95, and dJ149M18.1. Any naturally occurring isoform, allele, ortholog or variant is encompassed by the term NKp44 polypeptide (e.g., an NKp44 polypeptide 90%, 95%, 98% or 99% identical to SEQ ID NO: 51, or a contiguous sequence of at least 20, 30, 50, 100 or 200 amino acid residues thereof). The 276 amino acid residue sequence of human NKp46 is shown in SEQ ID NO: 51, which corresponds to NCBI accession number NP_004819, the disclosure of which is incorporated herein by reference. The human NKp46 mRNA sequence is described in NCBI accession number NM_004828, the disclosure of which is incorporated herein by reference.

“NKp30” refers to a protein or polypeptide encoded by an Ncr3 gene or by a cDNA prepared from such a gene. NKp30 has also been designated as NCR3 and CD337 (cluster of differentiation 337). Any naturally occurring isoform, allele, ortholog or variant is encompassed by the term NKp30 polypeptide (e.g., an NKp30 polypeptide 90%, 95%, 98% or 99% identical to SEQ ID NO:52, or a contiguous sequence of at least 20, 30, 50, 100 or 200 amino acid residues thereof). The 201 amino acid residue sequence of human NKp30 is shown in below in SEQ ID NO: 52, which corresponds to NCBI accession number NP_667341, the disclosure of which is incorporated herein by reference. The human NKp30 mRNA sequence is described in NCBI accession number NM_147130, the disclosure of which is incorporated herein by reference.

NKG2D is an activating receptor (transmembrane protein) belonging to the NKG2 family of C-type lectin-like receptors. NKG2D is encoded by KLRK1 gene in humans. NKG2D recognizes induced-self proteins from MIC and RAET1/ULBP families which appear on the surface of stressed, malignant transformed, and infected cells. “NKG2D” refers to a protein or polypeptide encoded by a KLRK1 gene or by a cDNA prepared from such a gene. NKG2D has also been designated as KLRK1, CD314 (cluster of differentiation 314), D12S2489E, KLR, NKG2-D, natural killer group 2D, killer cell lectin-like receptor K1, killer cell lectin like receptor K1. Any naturally occurring isoform, allele, ortholog or variant is encompassed by the term NKG2D polypeptide (e.g., an NKG2D polypeptide 90%, 95%, 98% or 99% identical to SEQ ID NO: 53, or a contiguous sequence of at least 20, 30, 50, 100 or 200 amino acid residues thereof). The 216 amino acid residue sequence of human NKG2D is shown in SEQ ID NO: 53, which corresponds to NCBI accession number NP_001186734, the disclosure of which is incorporated herein by reference. The human NKG2D mRNA sequence is described in NCBI accession number NM_007360, the disclosure of which is incorporated herein by reference.

DNAM-1 is a ~65 kDa glycoprotein expressed on the surface of amongst others NK cells. It is a member of the immunoglobulin superfamily containing 2 Ig-like domains of the V-set. DNAM-1 mediates cellular adhesion to other cells bearing its ligands, CD112 and CD155, and cross-linking DNAM-1 with antibodies causes cellular activation. “DNAM-1” refers to a protein or polypeptide encoded by a KLRK1 gene or by a cDNA prepared from such a gene. DNAM-1 has also been designated as CD226 (cluster of differentiation 226), DNAM-1, DNAM1, PTA1 and TLiSA1. Any naturally occurring isoform, allele, ortholog or variant is encompassed by the term DNAM-1 polypeptide (e.g., an DNAM-1 polypeptide 90%, 95%, 98% or 99% identical to SEQ ID NO: 54, or a contiguous sequence of at least 20, 30, 50, 100 or 200 amino acid residues thereof). The 336 amino acid residue sequence of human DNAM-1 is shown in SEQ ID NO: 54, which corresponds to NCBI accession number NP_006557, the disclosure of which is incorporated herein by reference. The human DNAM-1 mRNA sequence is described in NCBI accession number NM_006566, the disclosure of which is incorporated herein by reference.

As indicated above, CD16A is an immunoglobulin gamma Fc region receptor (FcγRIIIa) that is expressed on NK cells and through which NK cells recognize IgG that is bound to the surface of a pathogen-infected or TAA-expressing target cell. Any naturally occurring isoform, allele, ortholog or variant is encompassed by the term CD16A polypeptide (e.g., an CD16A polypeptide 90%, 95%, 98% or 99% identical to SEQ ID NO: 55, or a contiguous sequence of at least 20, 30, 50, 100 or 200 amino acid residues thereof). The 254 amino acid residue sequence of human CD16A is shown in SEQ ID NO: 55, which corresponds to UniProt accession no. P08637, the disclosure of which is incorporated herein by reference.

“SLAMF7” is a protein that in humans is encoded by the human SLAMF7 gene. Isoform 1 SLAMF7 mediates NK cell activation through a SH2D1A-independent extracellular signal-regulated ERK-mediated pathway. SLAMF7 has also been designated as CD319 (cluster of differentiation 319), 19A, CRACC, and CS1. Any naturally occurring isoform, allele, ortholog or variant is encompassed by the term SLAMF7 polypeptide (e.g., a SLAMF7 polypeptide 90%, 95%, 98% or 99% identical to SEQ ID NO: 56, or a contiguous sequence of at least 20, 30, 50, 100 or 200 amino acid residues thereof). The 335 amino acid residue sequence of human SLAMF7 is shown in SEQ ID NO: 56, which corresponds to UniProt accession no. Q9NQ25-1, the disclosure of which is incorporated herein by reference.

In one embodiment, a multispecific antigen binding protein as described herein comprises at least one antigen-binding region that is obtained/obtainable from a monoclonal antibody against NK cell activating receptor as is known in the art. In one embodiment, the at least one antigen-binding region at least comprises the six CDR sequences that are obtained/obtainable from a monoclonal antibody against NK cell activating receptor as is known in the art. In one embodiment, the at least one antigen-binding region at least comprises the variable light (V_{L}) domain and variable heavy (V_{H}) domain sequences that are obtained/obtainable from a monoclonal antibody against NK cell activating receptor as is known in the art. Many examples of monoclonal antibodies against NK cell activating receptor have been described in the art. Anti-NKp46 monoclonal antibodies are described WO 2011/086179, WO 2016/209021 and in Gauthier et al. (2019, Cell 177, 1701-1713) or in WO 2016/207278, such as NKp46-1, -2, -3, -4, -6 or -9. Anti-NKG2D monoclonal antibodies described WO 2009/077483, WO 2018/148447, WO 2019/157366, WO 2018/148445, WO 2018/152518 and WO 2019/195409, which include the heavy and light chain sequences of SEQ ID NO.'s: 16 and 20, respectively. Monoclonal antibodies against NKG2A are e.g. described in WO 2008/009545, WO 2009/092805, WO 2016/032334, WO 2020/094071 and WO 2020/102501. Monoclonal antibodies against NKp30 are e.g. described in WO 2020/172605. Monoclonal antibodies against DNAM-1 are e.g. described in WO 2013/140787. Examples of anti-SLAMF7 monoclonal antibodies include Elotuzumab and others described in US2018208653. Monoclonal antibodies against 4-1BB (CD137) are e.g. described in WO 2005/035584, WO 2006/088464 and US2006188439. Monoclonal antibodies against OX40 are e.g. described in WO 2007/062245, US2010136030, US2019100596, WO 2013/008171 and WO 2013/028231. Monoclonal antibodies against CD96 are e.g. described in WO 2019/091449. Monoclonal antibodies against CD160 are e.g. described in US2012003224 and US2013122006. Monoclonal antibodies against KIR2DS1-5 are e.g. described in WO 2016/031936.

In one embodiment therefore, a multispecific antigen binding protein as described herein comprises a combination of complementarity-determining regions (CDRs) CDR-H1, CDR-H2, CDR-H3, CDR-L1, CDR-L2 and CDR-L3 selected from the group consisting of: a) the CDR-H1 (SEQ ID NO: 24), CDR-H2 (SEQ ID NO: 25) and CDR-H3 (SEQ ID NO: 26) sequences as comprised in SEQ ID NO: 1, and the CDR-L1 (SEQ ID NO: 27), CDR-L2 (SEQ ID NO: 28) and CDR-L3 (SEQ ID NO: 29) sequences as comprised in SEQ ID NO: 2; and b) the CDR-H1, CDR-H2 and CDR-H3 sequences as comprised in SEQ ID NO: 3, and the CDR-L1, CDR-L2 and CDR-L3 sequences as comprised in SEQ ID NO: 20.

In one embodiment, a multispecific antigen binding protein as described herein comprises a combination of variable heavy (V_{H}) and variable light (V_{L}) domains selected from the group consisting of: a) the V_{H} sequence as comprised in SEQ ID NO: 1 and the V_{L} sequence as comprised in SEQ ID NO: 2; and b) the V_{H} sequence as comprised in SEQ ID NO: 3 and the V_{L} sequence as comprised in SEQ ID NO: 20.

In one embodiment, a multispecific antigen binding protein as described herein comprises a combination of heavy and light chains selected from the group consisting of: a) a heavy chain comprising the amino acid sequence of SEQ ID NO: 1 and a light chain comprising the amino acid sequence of SEQ ID NO: 2; and b) a heavy chain comprising the amino acid sequence of SEQ ID NO: 3 and a light chain comprising the amino acid sequence of SEQ ID NO: 20.

### Structure of the Multispecific Antigen Binding Protein

In one embodiment, in a multispecific antigen binding protein as described herein, at least NK cell-activating cytokine is conjugated to the region that has affinity for a surface antigen expressed on NK cells. The conjugation of the cytokine to the region is understood to mean that they are covalently linked to each other. The cytokine can be chemically cross-linked to the region, using a cross-linker for linking two proteinaceous molecules, as are well-known in the art. There are a number of commercially available crosslinking reagents for preparing protein or peptide bioconjugates. Many of these crosslinkers allow dimeric homo- or heteroconjugation of biological molecules through free amine or sulfhydryl groups in protein side chains. Other crosslinking methods involve coupling through carbohydrate groups with hydrazide moieties. For cross-linking an NK cell-activating cytokine to an NK cell-binding region it is preferred that cross-linkers with heterofunctional specificity are used. In one embodiment, the cross-linker comprises a flexible spacer, to provide flexibility or freedom of motion of the two moieties with respect to each other.

In one embodiment, however, the at least one NK cell-activating cytokine is conjugated to the region that has affinity for a surface antigen expressed on NK cells by being comprised in a single polypeptide chain. As the region that has affinity for a surface antigen expressed on NK cells can comprise two polypeptide chains, such as a dimeric Fc region of an antibody, in one embodiment, at least one polypeptide chain of the region that has affinity for a surface antigen expressed on NK cells forms a single polypeptide chain with the at least one NK cell-activating cytokine. In one embodiment, the NK cell-activating cytokine is linked to the polypeptide chain of the region that has affinity for a surface antigen expressed on NK cells through a flexible linker. In one embodiment, wherein the single polypeptide chain comprises more than one NK cell-activating cytokines, wherein optionally the NK cell-activating cytokines are linked to each other or the polypeptide chain of the region that has affinity for a surface antigen expressed on NK cells through a flexible linker. The flexible linker can be an immunoglobulin hinge region or can be linker as described herein below.

Thus, in one embodiment, a multispecific antigen binding protein as described herein comprises a single polypeptide chain that comprises in an N- to C-terminal order: i) a first NK cell-activating cytokine, ii) optionally a first flexible linker; iii) the polypeptide chain of the region that has affinity for a surface antigen expressed on NK cells; iv) optionally a second flexible linker; and v) a second NK cell-activating cytokine; and wherein preferably, the first and second NK cell-activating cytokine are different NK cell-activating cytokines. The first and second NK cell-activating cytokine are NK cell-activating cytokines can be selected from an IL21R agonist and a 4-1BB agonist as herein defined above.

In one embodiment, a multispecific antigen binding protein as described herein, further comprises at least one antigen-binding region that specifically binds an NK cell activating receptor. In one embodiment, the at least one antigen-binding region that specifically binds an NK cell activating receptor is conjugated to the region that has affinity for a surface antigen expressed on NK cells. Preferably, the antigen-binding region that specifically binds an NK cell activating receptor is conjugated to at least one polypeptide chain of the region that has affinity for a surface antigen expressed on NK cells. As described above, conjugation of the two regions is understood to mean that they are covalently linked to each other, which can be by chemically cross-linked to each other, using a cross-linker for linking two proteinaceous molecules, as are well-known in the art.

In one embodiment, however, the at least one antigen-binding region that specifically binds an NK cell activating receptor is conjugated to the region that has affinity for a surface antigen expressed on NK cells by being comprised in a single polypeptide chain. As the antigen-binding region can comprise two polypeptide chains, such as a V_{H} and a V_{L} chain, in one embodiment, at least one polypeptide chain in an antigen-binding region that specifically binds an NK cell activating receptor forms a single polypeptide chain with at least one polypeptide chain of the region that has affinity for a surface antigen expressed on NK cells. Likewise, as also the region that has affinity for a surface antigen expressed on NK cells can comprise two polypeptide chains, such as a dimeric Fc region of an antibody, in one embodiment, at least one polypeptide chain of the region that has affinity for a surface antigen expressed on NK cells forms a single polypeptide chain with the at least one polypeptide chain in an antigen-binding region that specifically binds an NK cell activating receptor.

Thus, in one embodiment, a multispecific antigen binding protein as described herein comprises a single polypeptide chain that comprises in an N- to C-terminal order: i) at least one polypeptide chain in the at least one antigen-binding region that specifically binds an NK cell activating receptor; ii) optionally a flexible linker; and iii) (at least one polypeptide chain of) the region that has affinity for a surface antigen expressed on NK cells. The flexible linker can be an immunoglobulin hinge region or can be linker as described herein below.

In one embodiment, the region that has affinity for a surface antigen expressed on NK cells is a dimeric immunoglobulin Fc region, wherein each of the two polypeptide chains of the Fc region is linked to a CH1 domain, each of which CH1 domains is linked to an immunoglobulin variable region that specifically binds an NK cell activating receptor. The dimeric immunoglobulin Fc region preferably is a dimer of an Fc region that binds CD16A as herein described above. The immunoglobulin variable region can be an scFv, a V_{H} domain, a V_{L} domain, or an immunoglobulin single variable domain (ISVD) such as a dAb, a V-NAR domain or a V_{H}H domain. In one embodiment, the immunoglobulin variable region that is linked to the CH1 domain is a V_{H} domain that is paired with a V_{L} domain linked to a CK or CA domain. In this embodiment, preferably, the V_{H} and V_{L} domains together specifically bind the NK cell activating receptor.

In one embodiment, the two immunoglobulin variable regions bind the same NK cell activating receptor, or wherein the two immunoglobulin variable regions each bind a different NK cell activating receptor. With respect to the specificity for an NK cell activating receptor, a multispecific antigen binding protein as described herein can thus be homodimeric, with two identical immunoglobulin variable regions that both bind the same NK cell activating receptor. Alternatively, a multispecific antigen binding protein as described herein can thus be heterodimeric with respect to the specificity for NK cell activating receptors, wherein each of the two immunoglobulin variable regions each bind a different NK cell activating receptor. In embodiments, wherein multispecific antigen binding protein is bispecific with respect to the NK cell activating receptors, it is preferred that one of the two immunoglobulin variable regions is an immunoglobulin single variable domain, while the other immunoglobulin variable region is not. Next, the assembly of heterodimeric antibody heavy chains can be accomplished by expressing two different antibody heavy chain sequences in the same cell, which may lead the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, U.S. Ser. No. 14/773,418, U.S. Ser. No. 12/811,207, U.S. Ser. No. 13/866,756, U.S. Ser. No. 14/647,480, U.S. Ser. No. 14/830,336 and WO2019/195409. For example, mutations can be made in the CH3 domain based on human IgG1 and incorporating distinct pairs of amino acid substitutions within a first polypeptide and a second polypeptide that allow these two chains to selectively heterodimerize with each other. For example, CH3 domains which comprise amino acid substitutions, wherein the CH3 domain interface of the antibody Fc region is mutated to create altered charge polarity across the Fc dimer interface such that co-expression of electrostatically matched Fc chains supports favorable attractive interactions thereby promoting desired Fc heterodimer formation, whereas unfavorable repulsive charge interactions suppress unwanted Fc homodimer formation.

In one embodiment, a “knob-into-holes” approach is used in which the CH3 domain interface of the antibody Fc region is mutated so that the antibodies preferentially form heterodimers (further including the attached light chains). These mutations create altered charge polarity across the Fc dimer interface such that coexpression of electrostatically matched Fc chains support favorable attractive interactions thereby promoting desired Fc heterodimer formation, whereas unfavorable repulsive charge interactions suppress unwanted Fc homodimer formation. For example, one heavy chain comprises a T366W substitution and the second heavy chain comprises a T366S, L368A and Y407V substitution, see, e.g. Ridgway et al (1996) Protein Eng., 9, pp. 617-621; Atwell (1997) J. Mol. Biol., 270, pp. 26-35; and WO2009/089004, the disclosures of which are incorporated herein by reference. In another approach, one heavy chain comprises a F405L substitution, and the second heavy chain comprises a K409R substitution, see, e.g., Labrijn et al. (2013) Proc. Natl. Acad. Sci. U.S.A., 110, pp. 5145-5150. In another approach, one heavy chain comprises T350V, L351Y, F405A, and Y407V substitutions and the second heavy chain comprises T350V, T366S, K392L, and T394W substitutions, see, e.g. Von Kreudenstein et al., (2013) mAbs 5:646-654. In another approach, one heavy chain comprises both K409D and K392D substitutions and the second heavy chain comprises both D399K and E356K substitutions, see, e.g. Gunasekaran et al., (2010) J. Biol. Chem. 285:19637-19646. In another approach, one heavy chain comprises D221E, P228E and L368E substitutions and the second heavy chain comprises D221R, P228R, and K409R substitutions, see, e.g. Strop et al., (2012) J. Mol. Biol. 420:204-219. In another approach, one heavy chain comprises S364H and F405A substitutions and the second heavy chain comprises Y349Tand, T394F substitutions, see, e.g. Moore et al., (2011) mAbs 3:546-557. In another approach, one heavy chain comprises a H435R substitution, and the second heavy chain optionally may or may not comprise a substitution, see, e.g. U.S. Pat. No. 8,586,713. When such hetero-multimeric antibodies have Fc regions derived from a human IgG2 or IgG4, the Fc regions of these antibodies can be engineered to contain amino acid modifications that permit CD16 binding. In some embodiments, the antibody may comprise mammalian antibody-type N-linked glycosylation at residue N297 (Kabat EU numbering).

In one preferred embodiment, a multispecific antigen binding protein as described herein comprises a dimeric immunoglobulin Fc region that is a dimer of an Fc region that binds CD16A as herein described above, wherein each of the two Fc polypeptide chains is operably linked to a Fab that specifically binds a NK cell activating receptor. Apart from the presence of the NK cell-activating cytokine(s), a multispecific antigen binding protein comprising such a dimeric Fc linked to two Fab's thus forms an immunoglobulin structure, such as a conventional IgG immunoglobulin.

A multispecific antigen binding protein as described herein, which comprises an antigen-binding region that specifically binds an NK cell activating receptor, thus further also comprises at least one NK cell-activating cytokine. In one embodiment, the at least one of the NK cell-activating cytokine is conjugated to the at least one antigen-binding region that specifically binds a NK cell activating receptor, or to the region that has affinity for a surface antigen expressed on NK cells. As indicated above conjugation of two proteinaceous entities is understood to mean that they are covalently linked to each other, which can be done by chemical cross-linking, using cross-linkers for linking two proteinaceous molecules, as are well-known in the art, which cross-linker can comprise a flexible spacer.

In one embodiment, however, the at least one NK cell-activating cytokine forms a single polypeptide chain with at least one of: i) at least one polypeptide chain in the at least one antigen-binding region that specifically binds a NK cell activating receptor; and, ii) (at least one polypeptide chain in) the region that has affinity for a surface antigen expressed on NK cells. In one embodiment, a flexible linker (as described below) is present between the agonist and the region defined in i) or ii).

In one embodiment, the at least one NK cell-activating cytokine forms a single polypeptide chain with at least one of: i) a light chain in at least one of the two Fab's that specifically bind a NK cell activating receptor; and, ii) at least one of the two Fc chains in the dimeric immunoglobulin Fc region. In one embodiment, a flexible linker (as described below) is present between the agonist and the light chain defined in i) or the Fc chain defined in ii).

In one embodiment, the at least one NK cell-activating cytokine is fused to at least one of: i) the N-terminus of the light chain in at least one of the two Fab's that specifically bind a NK cell activating receptor, optionally through a flexible linker; ii) the C-terminus of the light chain in at least one of the two Fab's that specifically bind a NK cell activating receptor, optionally through a flexible linker; iii) the N-terminus of the heavy chain in at least one of the two Fab's that specifically bind a NK cell activating receptor; and, iv) the C-terminus of the heavy chain in at least one of the two Fc chains in the dimeric immunoglobulin Fc region, optionally through a flexible linker, whereby the flexible linker can be as described below.

In one embodiment, wherein the multispecific antigen binding protein comprises a (homo- or hetero) dimeric antigen binding protein as described above, the dimer can comprise at least one NK cell-activating cytokine on only one of the two monomers in the dimer, or the dimer can comprise at least one NK cell-activating cytokine on each (i.e. both) of the two monomers in the dimer. Thus, in one embodiment, wherein the multispecific antigen binding protein comprises an immunoglobulin structure, at least one of the NK cell-activating cytokine can be present on at least one or on both sides of the immunoglobulin structure. In embodiments wherein at least one NK cell-activating cytokine is present on each of the two monomers in the dimer or immunoglobulin structure, the multispecific antigen binding protein can for example comprises an IL21R agonist on both monomers, a 4-1BB agonist on both monomers or, an IL21R agonist on a first monomer and a 4-1BB agonist on a second monomer. It is understood that when the multispecific antigen binding protein comprises heterodimeric heavy chains, the “knob-into-hole” technology as described above can be applied, wherein the CH3 domain of the first chain is modified to have a “protuberance” (“knob”) and the second chain is modified to have a corresponding “cavity” (“hole”).

Thus, in one embodiment, a multispecific antigen binding protein as described herein is heterodimeric with respect to at least one i) the antigen-binding regions that specifically bind an NK cell activating receptor; and ii) at least one fused NK cell-activating cytokine, and wherein the dimeric Fc region comprises different first and a second polypeptide chains comprising knob-into-hole modifications promoting association of the first and the second polypeptide chains of the Fc region.

Suitable linker-amino acid sequences for linking the various functional domains and regions in a multispecific antigen binding protein as described herein, are known in the art (e.g. from Chen et al., 2013, Adv Drug Deliv Rev. 65 (10): 1357-1369). Linker amino acid sequence can be rigid but are usually flexible. Flexible linkers are usually applied when the joined domains require a certain degree of movement or interaction. They are generally composed of small, non-polar (e.g. Gly) or polar (e.g. Ser or Thr) amino acids. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. Preferred flexible linkers have sequences consisting primarily of stretches of Gly and Ser residues (“GS” linker). An example of preferred (and widely used) flexible linker has the sequence of (GGGGS)ₙ (SEQ ID NO: 30). By adjusting the copy number “n”, the length of this GS linker can be optimized to achieve appropriate separation of the functional domains, or to maintain necessary inter-domain interactions. Specific examples of GS linkers include (GGGGS) 4 (SEQ ID NO: 31), GGGSGGG (SEQ ID NO: 32), GGSGGGGSGG (SEQ ID NO: 33) and G. Besides the GS linkers, many other flexible linkers have been designed for recombinant fusion proteins. These flexible linkers are also rich in small or polar amino acids such as Gly and Ser, but can contain additional amino acids such as Thr and Ala to maintain flexibility, as well as polar amino acids such as Lys and Glu to improve solubility, such as e.g. the flexible linkers KESGSVSSEQLAQFRSLD (SEQ ID NO: 34) and EGKSSGSGSESKST (SEQ ID NO: 35), that have been applied for the construction of a bioactive scFvs.

In one embodiment, a multispecific antigen binding protein as described herein is a multispecific antigen binding protein as exemplified herein, such as e.g. AVC-009, AVC-010, AVC-011, AVC-012, AVC-013, AVC-014 or AVC-015 (see Tables 1.1.1 and 1.1.2) or a derivative thereof wherein the anti-NKp46 variable heavy (V_{H}) and variable light (V_{L}) domains are replaced by variable heavy (V_{H}) and variable light (V_{L}) domains from another monoclonal antibody against an NK cell activating receptor.

In one embodiment, a multispecific antigen binding protein as described herein is a multispecific antigen binding protein comprising: a) a first heavy chain of a monoclonal antibody against an NK cell activating receptor as described above, wherein preferably a first NK cell activating cytokine is fused to the C-terminus of the first heavy chain, optionally through a flexible linker; b) a second heavy chain of the monoclonal antibody against the NK cell activating receptor, wherein preferably a second NK cell activating cytokine is fused to the C-terminus of the second heavy chain, optionally through a flexible linker; c) a first light chain and a second light chain of the monoclonal antibody against the NK cell activating receptor, wherein optionally the first or the second NK cell activating cytokine is fused to the C-terminus of the light chain, optionally through a flexible linker, wherein preferably the amino acid sequences of the Fc regions of the first and second heavy chains comprising knob-into-hole modifications promoting association of the first and the second heavy chains. In one embodiment, the first NK cell activating cytokine is a 4-1BB agonist as described above, preferably a fusion protein comprising three 4-1BBL ECD monomers fused together in a single polypeptide chain as described above, or a single 4-1BBL ECD monomer as described above. In one embodiment, the second NK cell activating cytokine is an IL21R agonist as described above, preferably an IL21R polypeptide as described above. In one embodiment, the monoclonal antibody against the NK cell activating receptor is a monoclonal antibody against NKp46 as described herein. In one embodiment, the Fc regions in the heavy chains comprise modification to decrease affinity for CD16A as described above, such as the “LALA” modifications.

In one embodiment, the multispecific antigen binding protein (AVC-009) comprises: a) a first heavy chain comprising an amino acid sequence with at least 95, 96, 97, 98, 99 or 100% sequence identity to SEQ ID NO: 11; b) a second heavy chain comprising an amino acid sequence with at least 95, 96, 97, 98, 99 or 100% sequence identity to SEQ ID NO: 12; and c) a first light chain and a second light chain comprising an amino acid sequence with at least 95, 96, 97, 98, 99 or 100% sequence identity to SEQ ID NO: 2.

In one embodiment, the multispecific antigen binding protein (AVC-010) comprises: a) a first heavy chain comprising an amino acid sequence with at least 95, 96, 97, 98, 99 or 100% sequence identity to SEQ ID NO: 13; b) a second heavy chain comprising an amino acid sequence with at least 95, 96, 97, 98, 99 or 100% sequence identity to SEQ ID NO: 14; and c) a first light chain and a second light chain comprising an amino acid sequence with at least 95, 96, 97, 98, 99 or 100% sequence identity to SEQ ID NO: 2.

In one embodiment, the multispecific antigen binding protein (AVC-011) comprises: a) a first and second heavy chain comprising an amino acid sequence with at least 95, 96, 97, 98, 99 or 100% sequence identity to SEQ ID NO: 15; and b) a first light chain and a second light chain comprising an amino acid sequence with at least 95, 96, 97, 98, 99 or 100% sequence identity to SEQ ID NO: 21.

In one embodiment, the multispecific antigen binding protein (AVC-012) comprises: a) a first and second heavy chain comprising an amino acid sequence with at least 95, 96, 97, 98, 99 or 100% sequence identity to SEQ ID NO: 39; and b) a first light chain and a second light chain comprising an amino acid sequence with at least 95, 96, 97, 98, 99 or 100% sequence identity to SEQ ID NO: 21.

In one embodiment, the multispecific antigen binding protein (AVC-013) comprises: a) a first and second heavy chain comprising an amino acid sequence with at least 95, 96, 97, 98, 99 or 100% sequence identity to SEQ ID NO: 18; and b) a first light chain and a second light chain comprising an amino acid sequence with at least 95, 96, 97, 98, 99 or 100% sequence identity to SEQ ID NO: 2.

In one embodiment, a multispecific antigen binding protein as described herein is a multispecific antigen binding protein comprising: a first and second polypeptide chain of an Fc region as described above, wherein preferably a first NK cell activating cytokine is fused to the N-terminus of at least one polypeptide chain of the Fc region, optionally through a flexible linker, and preferably a second NK cell activating cytokine is fused to the C-terminus of of at least one polypeptide chain of the Fc region, optionally through a flexible linker. In one embodiment, the first NK cell activating cytokine is an IL21R agonist as described above, preferably an IL21R polypeptide as described above. In one embodiment, the second NK cell activating cytokine is a 4-1BB agonist as described above, preferably a fusion protein comprising three 4-1BBL ECD monomers fused together in a single polypeptide chain as described above, or a single 4-1BBL ECD monomer as described above. In one embodiment, the Fc regions in the heavy chains comprise modification to decrease affinity for CD16A as described above, such as the “LALA” modifications. In one embodiment, the multispecific antigen binding protein (AVC-014) comprises: a) a first and second polypeptide chain comprising an amino acid sequence with at least 95, 96, 97, 98, 99 or 100% sequence identity to SEQ ID NO: 19. In one embodiment, the multispecific antigen binding protein (AVC-015) comprises: a) a first and second polypeptide chain comprising an amino acid sequence with at least 95, 96, 97, 98, 99 or 100% sequence identity to SEQ ID NO: 40.

### Biological Activities of the Multispecific Antigen Binding Protein

A multispecific antigen binding protein as described herein can have a one or more biological activities, including e.g. antigen binding, binding to an NK cell, the ability to direct an NK cell to a target cell, activating an NK cell, including inducing hyper-functionality of the NK cell, and/or the ability to elicit lysis of target cell by the (activated/hyper-functional) NK cell.

In one embodiment, a multispecific antigen binding protein as described herein cause an increase in at least one NK cell activity selected from CD107a degranulation, CD107 or CD69 expression, IFNy production, NK cell proliferation and NK cell cytotoxicity, whereby preferably, the increase is at least a factor 0.1, 0.2, 0.5, 1.0, 2.0, 5.0, 10, 20 50 or 100 higher as compared to the increase achieved with the same effector:target cell ratio, with the same NK cells and target cells that are not brought into contact with the multispecific antigen binding protein.

In one embodiment, a multispecific antigen binding protein as described herein cause an increase in at least one NK cell activity selected from CD107a degranulation, CD107 or CD69 expression, IFNy production, NK cell proliferation and NK cell cytotoxicity, whereby preferably, the increase is at least a factor 0.1, 0.2, 0.5, 1.0, 2.0, 5.0, 10, 20 50 or 100 higher as compared to the increase achieved with the same effector:target cell ratio, with the same NK cells and target cells that are brought into contact (under otherwise identical conditions) with a reference antigen binding protein or with a reference feeder cell line.

In one embodiment, the reference antigen binding protein is a conventional human IgG1 monoclonal antibody that binds to the same NK cell activating receptor, preferably, that binds to the same epitope, more preferably that has same NK cell activating receptor-specific antigen-binding region(s) as the multispecific antigen binding protein.

In one embodiment, the reference antigen binding protein is a (multispecific) antigen binding protein comprising at least one antigen-binding region that binds to the same NK cell activating receptor, preferably, that binds to the same epitope, more preferably that has same NK cell activating receptor-specific antigen-binding region(s) as the multispecific antigen binding protein, and comprising at least one NK cell-activating cytokine cytokine other than an IL21R agonist, preferably, at least one NK cell-activating cytokine other than an IL21R agonist and a 4-1BB agonist. The NK cell-activating cytokine other than at least one of an IL21R agonist and a 4-1BB agonist, can be an IL-15 receptor agonist, such as IL15, for example a human modified IL-15 cross-linker as described in US2018282386 and Vallera et al. (2016, Clin Cancer Res.; 22 (14): 3440-3450).

In one embodiment, the reference antigen binding protein is an NK cell engager, such as e.g. described in WO2016/207278, WO 2018/148445, WO2018/152518, WO2019195409 US2018282386, Vallera et al. (2016, supra) and Demaria et al. (2021, supra). One example of a (multispecific) reference antigen binding protein is for example AVC-006 described in the Examples herein, comprising one HER2-binding region and one NKG2D-binding region.

In one embodiment, the reference feeder cell line is the (irradiated) K562 feeder cell line that has been modified to express membrane bound IL-21 (mbIL-21) and 4-1BB ligand (FC21 feeder cells) as described by Denman et al. (2012, supra).

Assays which detect the expression of an NK activation marker, or which detects NK cell cytotoxicity, or for detecting NK cell activation and cytotoxicity assays (e.g. short and long term cytotoxicity assays) are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; El-Sherbiny et al, (2007) Cancer Research 67 (18): 8444-9; Nolte-′t Hoen et al, (2007) Blood 109:670-673); WO 2016/207278 and WO 2018/148445.

In one embodiment, a multispecific antigen binding protein as described herein has the ability to induce hyper-functionality (or a hyper-functional phenotype) in an NK cell or in a population of NK cells. A hyper-functional NK cell phenotype is herein understood as a phenotype that has one or more of the features of the phenotype that is obtained by expanding NK cells obtained from donors ex vivo by co-culturing them with irradiated K562 feeder cells modified to express membrane bound IL-21 (mbIL-21) and 4-1BB ligand (FC21 feeder cells) as described by Denman et al. (2012, supra). Thus, in one embodiment, ex vivo expansion of donor NK cells by co-culturing (e.g. for 7, 14 or 21 days), with a multispecific antigen binding protein as described herein, produces a population of NK cells having one or more (or preferably all) of the features selected from the group: a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated FC21 feeder cells; b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of expanded NK cells obtained by ex vivo expansion by co-culturing with irradiated FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on NK cells obtained upon ex vivo expansion in the presence of FC21 feeder cells; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold of the secretion of the cytokine by NK cells obtained upon ex vivo expansion in the presence of FC21 feeder cells; and, e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of NK cells obtained upon ex vivo expansion in the presence of FC21 feeder cells. Protocols for ex vivo expansion of donor NK cells and assays for determining fold expansion, telomere length increase, expression level of NK cell activating receptors, cytokine secretion and cytotoxicity (e.g. short term or long term cytotoxicity assays) are described in Denman et al. (2012, supra) and in the Examples herein.

### Pharmaceutical Compositions

In a further aspect, the invention relates to a pharmaceutical composition comprising a multispecific antigen binding protein as described herein, and a pharmaceutically acceptable carrier (excipient). The pharmaceutically acceptable carrier such as an adjuvant, or vehicle, is for administration of the polypeptide to a subject. Said pharmaceutical composition can be used in the methods of treatment described herein below by administration of an effective amount of the composition to a subject in need thereof. The term “subject”, as used herein, refers to all animals classified as mammals and includes, but is not restricted to, primates and humans. The subject is preferably a male or female human of any age or race.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration (see e.g. “Handbook of Pharmaceutical Excipients”, Rowe et al eds. 7th edition, 2012, www.pharmpress.com). The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter ions such as sodium; metal complexes (e.g. Zn²⁺-protein complexes); and/or non-ionic surfactants such as TWEEN™, PLURONICS™ or polyethylene glycol (PEG).

Supplementary active compounds can also be incorporated into the pharmaceutical composition of the invention. Thus, in a particular embodiment, the pharmaceutical composition of the invention may also contain more than one active compound as necessary for the particular indication being treated, preferably those with complementary activities that do not adversely affect each other. For example, it may be desirable to further provide a chemotherapeutic agent, a cytokine, an analgesic agent, a thrombolytic or an immunomodulating agent, e.g. an immunosuppressive agent or an immunostimulating agent. The effective amount of such other active agents depends, among other things, on the amount of the polypeptide of the invention present in the pharmaceutical composition, the type of disease or disorder or treatment, etc.

In one embodiment, the polypeptide of the invention is prepared with carriers that will protect said compound against rapid elimination from the body, such as a controlled release formulation, including implants and microencapsulated delivery systems, e.g. liposomes. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions, including targeted liposomes can also be used as pharmaceutically acceptable carriers. These can be prepared according to methods known to those skilled in the art, for example, as described in U.S. Pat. No. 4,522,811 or WO2010/095940.

The administration route of the polypeptide of the invention can be parenteral. The term “parenteral” as used herein includes intravenous, intra-arterial, intralymphatic, intraperitoneal, intramuscular or subcutaneous. The intravenous or intramuscular forms of parenteral administration are preferred. By “systemic administration” is meant intravenous, intraperitoneal and intramuscular administration. The amount of the polypeptide required for therapeutic or prophylactic effect will, of course, vary with the polypeptide chosen, the nature and severity of the condition being treated and the patient. In addition, the polypeptide may suitably be administered by pulse infusion, e.g., with declining doses of the polypeptide. Preferably the dosing is given by injections, most preferably intravenous, intramuscular or subcutaneous injections, depending in part on whether the administration is brief or chronic.

Thus, in a particular embodiment, the pharmaceutical composition of the invention may be in a form suitable for parenteral administration, such as sterile solutions, suspensions or lyophilized products in the appropriate unit dosage form. Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, CremophorEM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol or sodium chloride in the composition.

Prolonged absorption of the injectable compositions can be brought about by including in the composition an agent which delays absorption, for example, aluminium monostearate and gelatin.

Sterile injectable solutions can be prepared by incorporating the active compound (e.g a polypeptide of the invention) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

In a particular embodiment, said pharmaceutical composition is administered via intravenous (IV), intramuscular (IM) or subcutaneous (SC) route. Adequate excipients can be used, such as bulking agents, buffering agents or surfactants. The mentioned formulations will be prepared using standard methods for preparing parenterally administrable compositions as are well known in the art and described in more detail in various sources, including, for example, “Remington: The Science and Practice of Pharmacy” (Ed. Allen, L. V. 22nd edition, 2012, www.pharmpress.com).

It is especially advantageous to formulate the pharmaceutical compositions, namely parenteral compositions, in dosage unit form for ease administration and uniformity of dosage. Dosage unit form as used herein refers to physically discrete units suited as unitary dosages for the subject to be treated; each unit containing a predetermined quantity of active compound (polypeptide of the invention) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Generally, for the prevention and/or treatment of the diseases and disorders mentioned herein and depending on the specific disease or condition to be treated and its severity, the potency of the specific polypeptide of the invention to be used, the specific route of administration and the specific pharmaceutical formulation or composition used, the polypeptide of the invention will generally be administered in the range of from 0.001 to 1,000 mg/kg body weight/day, preferably about 0.01 to about 100 mg/kg body weight/day, most preferably from about 0.05 to 10 mg/kg body weight/day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. The pharmaceutical compositions can be included in a container, pack, or dispenser together with instructions for administration.

### Therapeutic Use

In another aspect there is provided a multispecific antigen binding protein as described herein for use as a medicament. In one embodiment, the multispecific antigen binding is used as protein as described herein is used as an active ingredient, component or substance in a medicament.

In one aspect, the invention pertains to a use of a multispecific antigen binding protein as described herein for the manufacture of a medicament, e.g. a pharmaceutical preparation comprising the multispecific antigen binding protein as an active ingredient, for the treatment, prevention or diagnosis of a disease in a subject in need thereof.

In one aspect, the invention pertains to a multispecific antigen binding protein as described herein, or a pharmaceutical preparation comprising the multispecific antigen binding protein as an active ingredient, for use in the treatment, prevention or diagnosis of a disease in a subject in need thereof.

In one aspect, the invention pertains to a method for the treatment of a disease in a subject in need thereof, wherein the method comprises the step of administering to the subject (an effective amount of) a multispecific antigen binding protein as described herein, or a pharmaceutical preparation comprising the multispecific antigen binding protein as an active ingredient.

The disease to be treated, prevented or diagnosed using the multispecific antigen binding protein can be a cancer, an infectious disease, an inflammatory disease or an autoimmune disease.

In one embodiment, the disease to be treated, prevented or diagnosed using the multispecific antigen binding protein is a cancer, e.g. a cancer as described below.

In one embodiment, the invention pertains to a method for enhancing anti-tumor activity of an NK cell in a subject, the method comprising the step of administering to the subject a multispecific antigen binding protein as described herein, or a pharmaceutical preparation comprising the multispecific antigen binding protein as an active ingredient. In one embodiment, the subject has cancer, e.g. a cancer as described below.

In one embodiment, the invention pertains to a method for expanding and/or induce hyper-functionality NK cells a in a subject, the method comprising the step of administering to the subject a multispecific antigen binding protein as described herein, or a pharmaceutical preparation comprising the multispecific antigen binding protein as an active ingredient. The fold expansion and the hyper-functionality preferably is as herein described above. In one embodiment, the subject has cancer, e.g. a cancer as described below.

Subjects having cancer, often present with lower numbers of NK cells and/or with exhausted NK cells. The multispecific antigen binding proteins of the invention can thus be advantageously used to expand the numbers of NK cells and/or to induce hyper-functionality of the NK cells in a subject suffering from cancer. A further advantage of the hyper-functionality of NK cells as induced by the multispecific antigen binding protein of the invention, includes their increased secretion of cytokines such as TNF-α, IFN-γ and IL-6, which help shape adaptive immune response involving DCs and T cells. Indeed NK cells have been reported to promote the recruitment to the tumor micro environment of a DC subset specializing in the crosspresentation of tumor antigens to CD8+ T cells, suggesting a crucial role for NK cells in the potentiation of antitumor CD8+ T cell responses (Bottcher et al., Cell, 2018. 172:1022-1037; and Barry et al., Nat. Med. 2018. 24:1178-1191). The contribution of NK cells to the orchestration of antitumor T-cell responses has also been confirmed experimentally in mice, demonstrating that, in addition to their direct effector functions, NK cell can promote T-cell responses and long-lasting immune control of tumors (Bonavita et al., Immunity 2020. 53:1215-1229).

In one embodiment, the cancer to be treated, prevented or diagnosed using the multispecific antigen binding protein is a cancer selected from the group consisting of: carcinoma, including that of the bladder, head and neck, breast, colon, kidney, liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkin's lymphoma, non-Hodgkin's lymphoma, hairy cell lymphoma and Burkett's lymphoma; hematopoietic tumors of myeloid lineage, including acute and chronic myelogenous leukemias and promyelocytic leukemia; tumors of mesenchymal origin, including fibrosarcoma and rhabdomyosarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) preferably of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukemia (T-Lbly/T-ALL).

In one embodiment, a multispecific antigen binding protein as described herein can be used as a monotherapy (i.e. without other therapeutic agents). In another embodiment, a multispecific antigen binding protein as described herein can be used in combined treatments.

In one embodiment, a multispecific antigen binding protein as described herein is used in combination with another immunotherapy, e.g. a cellular immunotherapy. The multispecific antigen binding protein can thus be used in combination with the adoptive transfer of immune cells, including the adoptive transfer of T cells, e.g. CAR T cells, or NK cells. The NK cells can e.g. be enriched or expanded by methods known in the art or can be ex vivo NK cells as herein described.

In one embodiment, a multispecific antigen binding protein as described herein can be used in combined treatments with one or more other therapeutic agents. The additional therapeutic agent or agents will normally be administered in amounts and treatment regimens typically used for that agent in a monotherapy for the particular disease or condition being treated. Such therapeutic agents when used in the treatment of cancer, include, but are not limited to anti-cancer agents and chemotherapeutic agents. Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine flutamide, drogenil, butocin, carmofur, razoxane, sizofilann, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and luteinizing hormone releasing factor.

An additional class of agents that may be used as part of a combination therapy in treating cancer is immune checkpoint inhibitors. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG3, (v) B7-H3, (vi) B7-H4, and (vii) TIM3. Yet other agents that may be used as part of a combination therapy in treating cancer are cytotoxic monoclonal antibodies against TAA, including cytotoxic monoclonal antibody against a TAA known in the art such as: trastuzumab (to HER2), pertuzumab (to HER2), rituximab (to CD20), tositumomab (to CD20), ibritumomab (to CD20), obinutuzumab (to CD20), ofatumumab (to CD20), alemtuzumab (to CD52), blinatumomab (to CD19), inebilizumab (to CD19), tafasitamab (to CD19), daratumumab (to CD38), isatuximab (to CD38), polatuzumab (to CD79b), dinutuximab (to GD2), naxitamab (to GD2), bevacizumab (to VEGF-A), elotuzumab (to SLAMF7), enfortumab (to nectin-4) sacituzumab (to TROP2), mogamulizumab (to CCR4), ipilimumab (to CTLA-4), tremelimumab (to CTLA-4), durvalumab (to PD1), pidilizumab (to PD-1), pembrolizumab (to PD-1), nivolumab (to PD-1), cemiplimab (to PD-1), avelumab (to PD-L1), cetuximab (to EGFR), necitumumab (to EGFR), panitumumab (to EGFR), olaratumab (to PDGFRα) and ramucirumab (to VEGFR2).

In some embodiments the administration of the multispecific antigen binding protein and the other therapeutic agent can elicit an additive or synergistic effect on immunity and/or on therapeutic efficacy.

In one embodiment, a multispecific antigen binding protein as described herein is used as at least one of an neoadjuvant therapy and an adjuvant therapy, in addition to a primary therapy comprising e.g. surgery and/or radiation therapy. As an neoadjuvant therapy, the multispecific antigen binding protein is administered before the primary treatment, e.g. to help reduce the size of a tumor (such that less extensive surgery and/or radiation therapy is required), kill cancer cells that have spread (e.g. micrometastatic disease) and/or reduce the risk of tumor cells spreading post-surgery. As an adjuvant therapy, the multispecific antigen binding protein is administered after the primary treatment, e.g. to treat minimal residual disease (destroy remaining cancer cells). The use of the multispecific antigen binding protein as an neoadjuvant therapy and/or an adjuvant therapy lowers relapse rates. In the neoadjuvant therapy and/or adjuvant therapy, the multispecific antigen binding protein can be used as monotherapy or in in combined treatments as described above.

### Ex Vivo Methods

In a further aspect, the invention relates to methods wherein a multispecific antigen binding protein as described herein is used for ex vivo (in vitro) treatment of an NK cell or a population of NK cells. The method can be a method for at least one of expanding, preactivating, activating, enhancing cytotoxicity and/or cytokine production, and inducing a hyper-functional phenotype as defined above. The methods at least comprise the step of contacting an NK cell or a population thereof, with a multispecific antigen binding protein as described herein or with a composition comprising the multispecific antigen binding protein.

An NK cell or a population of NK cells for ex vivo treatment can be enriched from peripheral blood mononuclear cells (PBMCs). Methods for enrichment and ex vivo treatment of NK cells from PBMCs are e.g. described in Denman et al. (PLoS One. 2012; 7 (1): e30264) and in US2020/0061115. For example, NK cells enriched from PBMCs can be seeded at 0.1×10⁶ NK cells/mL in SCGM (CellGenix, Portsmouth, N.H.), supplemented with 10% FBS, 2 mM Glutamax, 100 U/mL IL-2 (Peprotech, Rocky Hill, N.J.) and 1, 2, 5, 10, 20, 50, 100, 200, 500, 1000 μg/mL of one or more multispecific antigen binding proteins as described herein. Media with supplements can be refreshed every 2-3 days after day 5.

It is understood that the duration of the contact between the NK cells and a multispecific antigen binding protein as described herein (i.e. the duration of the preactivation or activation (i.e., the duration of expanding, preactivating, activating, enhancing cytotoxicity and/or cytokine production and inducing a hyper-functional phenotype) can be for any length of time necessary to achieve the desired phenotype of the NK cells. For example, the contact can be as little as 1 minute or as much as 7 days (for example, culturing the NK cells in the presence of a multispecific antigen binding protein as described herein for 7 days). In one embodiment of the method, the NK cells are contacted with the multispecific antigen binding protein for 5, 6, 7, 8, 9, 10, 11, 12, 13, 14, 15, 16, 17, 18, 19, 20, 21, 22, 23, 24, 36, or 48 hours. In one embodiment of the method, the NK cells are contacted with the multispecific antigen binding protein for 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 days.

In one embodiment, the ex vivo treated (expanded) NK cells have one or more features selected from: a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated FC21 feeder cells; b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of expanded NK cells obtained by ex vivo expansion by co-culturing with irradiated FC21 feeder cells; c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on NK cells obtained upon ex vivo expansion in the presence of FC21 feeder cells; d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold of the secretion of the cytokine by NK cells obtained upon ex vivo expansion in the presence of FC21 feeder cells; and, e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of NK cells obtained upon ex vivo expansion in the presence of FC21 feeder cells.

In yet a further aspect, the invention pertains to a method for the treatment of a disease in a subject in need thereof, wherein the method comprises the step of administering to the subject (an effective amount of) NK cells obtained in the above method for ex vivo treatment of an NK cell or a population of NK cells. Once a sufficient number of NK cells with the desired (hyper-functional) phenotype has been expanded by the ex vivo treatment, the NK cells can be administered to a subject in need thereof.

In one embodiment, the method for the treatment comprises the administration of the ex vivo treated NK cells in combination with a multispecific antigen binding protein as described herein, or a pharmaceutical preparation comprising the multispecific antigen binding protein as an active ingredient.

In one embodiment, the method for the treatment comprises the administration of the ex vivo treated NK cells in combination with another NK cell engager, such as e.g. described in WO2016/207278, WO 2018/148445, WO2018/152518, WO2019195409 US2018282386, Vallera et al. (2016, supra) and Demaria et al. (2021, supra), or with a multispecific antigen binding protein as described in the co-pending application by the same applicant with reference no. P6110749EP. One example of another NK cell engager is for example AVC-006 described in the Examples herein, comprising one HER2-binding region and one NKG2D-binding region. In a further embodiment, the ex vivo treated NK cells can be used in combination with the other engager and with a multispecific antigen binding protein as described herein.

The disease to be treated can be a cancer, an infectious disease, an inflammatory disease or an autoimmune disease, as described above. Preferably, the disease to be treated is a cancer, as described above.

In one embodiment, the ex vivo treated NK are autologous to the subject. In another embodiment, the ex vivo treated NK are allogeneic, e.g. derived from donor PBMCs.

### Nucleic Acids, Host Cells and Methods for Producing Multispecific Antigen Binding Protein

In one aspect, the invention relates to a nucleic acid molecule comprising one or more nucleotide sequences encoding a polypeptide chain of a multispecific antigen binding protein as described herein. The nucleotide sequence encoding such a polypeptide chain preferably encodes a signal peptide operably linked to the polypeptide chain. A nucleic acid molecule comprising one or more of the nucleotide sequences encoding a polypeptide chain, further preferably comprises regulatory elements for (or conducive to) the expression of the polypeptide chain in an appropriate host cell, which regulatory elements are operably linked to the nucleotide sequence.

In one aspect, the invention relates to a host cell comprising the nucleic acid molecule comprising one or more nucleotide sequences encoding a polypeptide chain of a multispecific antigen binding protein as described herein. In one embodiment, the host cell is an isolated cell or a cultured cell. Among the host cells that may be employed are prokaryotes, yeast or higher eukaryotic cells. Prokaryotes include gram-negative or gram-positive organisms, for example Escherichia coli or bacilli. Suitable yeast cells include Saccharomyces cerevisiae and Pichia pastoris. Higher eukaryotic cells include insect cells and established cell lines of mammalian origin. Examples of suitable mammalian host cell lines include the COS-1, COS-7 line of monkey kidney cells (Gluzman et al., 1981, Cell 23:175), L cells, HEK 293 cells, C127 cells, 3T3 cells, Chinese hamster ovary (CHO) cells, Hela cells, BHK cell lines, e.g. BHK21, BSC-1, Hep G2, 653, SP2/0, and the CVI/EBNA cell line derived from the African green monkey kidney cell line CVI as described by McMahan et al. (1991, EMBO J. 10:2821). The host cell may be any suitable species or organism capable of producing N-linked glycosylated polypeptides, e.g. a mammalian host cell capable of producing human or rodent IgG type N-linked glycosylation. Appropriate cloning and expression vectors for use with bacterial, fungal, yeast, and mammalian cellular hosts are described by Pouwels et al. (Cloning Vectors: A Laboratory Manual, Elsevier, N.Y., 1985). Host cells comprising the nucleic acid molecule of the invention can be cultured under conditions that promote expression of the polypeptide.

Thus, another aspect the invention relates to a method for producing a multispecific antigen binding protein as described herein. The method preferably comprises culturing a host cell as described above such that one or more nucleotide sequences are expressed and the multispecific antigen binding protein is produced. The method preferably comprises the step of cultivating a host cell comprising one or more of the nucleotide sequences encoding a polypeptide chain of the multispecific antigen binding protein. The host cell is preferably cultured under conditions conducive to expression of the one or more polypeptide chains. The method can further comprise the step of recovering the multispecific antigen binding protein. The multispecific antigen binding protein can be recovered by conventional protein purification procedures, including e.g. protein A-Sepharose, hydroxylapatite chromatography, gel electrophoresis, dialysis, size exclusion chromatograpy or affinity chromatography, using e.g. strepavidin/biotin (see e.g. Low et al., 2007, J. Chromatography B, 848:48-63; Shukla et al., 2007, J. Chromatography B, 848:28-39).

In a further aspect, the invention relates to a method for producing a pharmaceutical composition comprising a multispecific antigen binding protein as described herein, the method comprising the steps of a) producing the multispecific antigen binding protein in a method as defined above; and b) formulating the multispecific antigen binding protein with a pharmaceutically acceptable carrier as defined above, to obtain a pharmaceutical composition.

The present invention has been described above with reference to a number of exemplary embodiments as shown in the drawings and examples. Modifications and alternative implementations of some parts or elements are possible and are included in the scope of protection as defined in the appended claims.

### DESCRIPTION OF THE FIGURES

FIG. 1. Schematic depiction of multispecific antigen binding protein and control proteins AVC-009 to AVC-015 (see Example 1 for sequence details). AVC-009 to AVC-010 are heterodimeric. AVC-011 to AVC-015 are homodimeric. NKp46Fab=Fab fragment specific for NKp46; TrasFc=trastuzumab Fc region; 41BBLT=4-1BB ligand trimer; 41BBLM=4-1BB ligand monomer; CD16-=L234A/L235A modifications in the Fc region for reduced binding to CD16.

FIG. 2. Ex vivo expansion of donor NK cells stimulated with the multispecific antigen binding proteins AVC-009 (AVC9), AVC-011 (AVC11) or AVC-014 (AVC14). FC21 is the reference expansion method using irradiated FC21 feeder cells.

FIG. 3. Cytotoxicity against MDA-MB-231 target cells of NK cells that have been stimulated for one week with the multispecific antigen binding proteins AVC-009 (AVC9), AVC-011 (AVC11) or AVC-014 (AVC14), or FC21 feeder cells as a reference. All three of AVC-009, AVC-011 and AVC-014 induce strong cytotoxicity against MDA-MB-231 target cells. The cytotoxicity of NK cells expanded in the presence of AVC-009 or AVC-014 does not significantly differ from that of NK cell stimulated by the reference FC21 feeder cells.

FIG. 4. Shown here is a viSNE analysis as described in Example 4.3, using 30 markers, which shows the population distribution of cells from different expansion methods. AVC11, AVC14, AVC9 and FC21 are after one weeks of expansion as described in Example 3.5, in the presence of the multispecific antigen binding proteins AVC-009, AVC-011 or AVC-014, or FC21 feeder cells, resp. The color mapping indicates CD56 or Ki67 expression, which is used to identify NK cells. Compare to the control condition of unexpanded NK cells, all 4 expansion methods result in a enrichment of highly CD56-expressing NK cells which have a very similar phenotype.

FIG. 5. Up- or down regulation of NK cell-surface markers as indicated after one weeks of expansion of NK cells as described in Example 3.5, in the presence of the multispecific antigen binding proteins AVC-009, AVC-011 or AVC-014, or FC21 feeder cells (FC21), resp. Fold change (Log 2) is compared to the control condition of unexpanded NK cells.

FIG. 6: Comparison of the proliferation of NK cells induced by culturing the cells as described in Example 3.5 in the presence of the multispecific antigen binding proteins AVC9 (IL-21 and 4-1BBL), AVC81 (IL-21) or AVC82 (4-1BBL).

### EXAMPLES

### Example 1 Preparation of Multispecific Antigen Binding Proteins

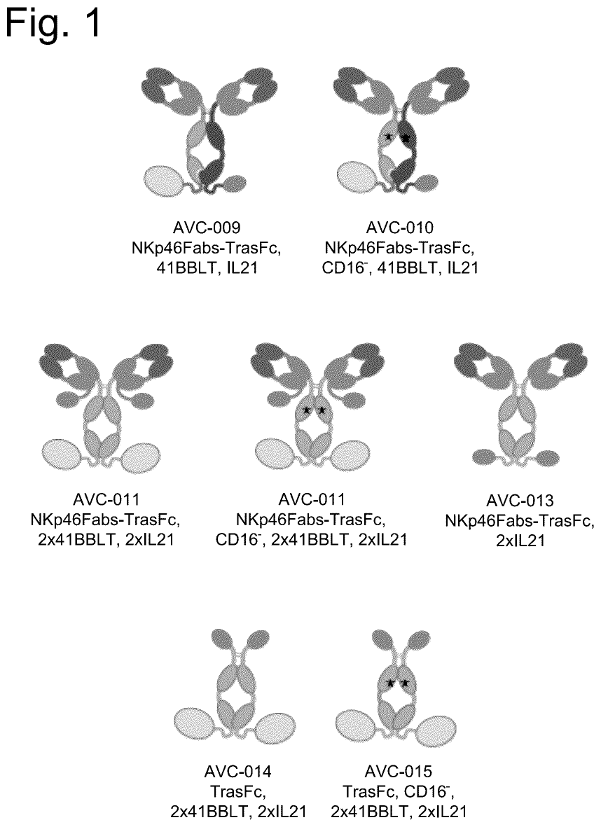

Coding sequences were generated for the multispecific antigen binding proteins and control proteins, including proteins AVC-009 to AVC-015 as schematically depicted in FIG. 1, the amino acid sequences of which are depicted in Tables 1.1.1 and 1.1.2.

Expression constructs for expression of the coding sequences in the HEK293-E-253 cell line were prepared using standard molecular biological materials and techniques. Briefly, coding sequences were generated by direct synthesis and/or by PCR. PCR was performed using the PrimeSTAR MAX DNA polymerase (Takara, #R045A) and PCR products were purified from 1% agarose gel using the NucleoSpin gel and PCR clean-up kit (Macherey-Nagel, #740609.250). Once purified the PCR products were quantified prior to the In-Fusion ligation reaction which was performed as described in the manufacturer's protocol (ClonTech, #ST0345). The plasmids were obtained after a miniprep preparation. Plasmids were then sequenced for sequence confirmation before being transfecting into the HEK293-E-253 cell line.

All used buffers were made using Versylene (endotoxin-free and sterile) water. Endotoxin was removed from instruments by incubation with 0.1 M NaOH for at least 16 hours. Line clearance was performed at start and end of the production.

HEK293E-253 cells were transfected with endotoxin-free plasmid DNA using the rPEx® technology. Six days post transfection conditioned medium containing recombinant protein was harvested by centrifugation and filtration over a 0.22 μm bottletop filter. A 100 μl sample was stored at 4° C. To avoid binding of a-specific proteins or cell fragments to the MabSelect PrismA resin, the sample was conditioned with 40 ml, 5 M NaCl per 1 L medium. This results in an increase of the NaCl concentration with 0.2 M.

A HiScreen Fibro PrismA column was equilibrated in 20 mM Tris 150 mM NaCl pH 7.8. The recombinant protein in the conditioned medium was loaded onto the column using a Teledyne ASX-560 autosampler and a-specific bound proteins were removed by washing the column with 20 mM Tris 1 M NaCl pH 7.8 and with 20 mM citrate 150 mM NaCl pH 5.0. The bound recombinant protein was eluted by a 12 CV block gradient to 20 mM citrate 150 mM NaCl pH 3.5 and a 6 CV block gradient to 20 mM citrate 150 mM NaCl pH 3.0. The eluate was directly in-line neutralized by mixing-in of 1 M Tris pH 8.0 for62ot hat62zationn to pH 7 in a 1.0/0.2 ratio and 12.5 ml fractions were collected. Between injections, the HiScreen Fibro PrismA column was regenerated using 5 CV 0.5 M NaOH, 2 CV 1 M Tris pH 8.0 and equilibrated in 20 mM Tris 150 mM NaCl pH 7.8. The Teledyne autosampler was cleaned with 15 ml 0.1 M NaOH and equilibrated in PBS.

The HiTrap Fibro PrismA pool was concentrated to 2-3 ml per injection on a Superdex200 increase 16/40 column using an Amicon 30 kDa spin filter. Aggregates in the concentrated pool were removed by filtration over a 0.22 μm syringe filter. The concentrated sample was stored at 4° C. before gelfiltration.

The recombinant protein products were analyzed by Labchip capillary electrophoresis and LAL assay.

TABLE 1.1.1
Overview of the multispecific antigen binding proteins AVC-009-AVC-015 and reference protein AVC-006.

| Construct | Heavy chain 1 | | Heavy chain 2 | | Light chain 1 | | Light chain 2 | |
|---|---|---|---|---|---|---|---|---|
| AVC-009 | Anti-NKp46 | SEQ ID | Anti-NKp46 | SEQ ID | Anti-NKp46 | SEQ ID | Anti-NKp46 | SEQ ID |
| | HC1-4-1BBL | NO: 11 | HC2-IL21 | NO: 12 | LC | NO: 2 | LC | NO: 2 |
| | trimer fusion | | fusion | | | | | |
| AVC-010 | Anti-NKp46 | SEQ ID | Anti-NKp46 | SEQ ID | Anti-NKp46 | SEQ ID | Anti-NKp46 | SEQ ID |
| | CD16⁻ HC1-4- | NO: 13 | CD16⁻ HC2- | NO: 14 | LC | NO: 2 | LC | NO: 2 |
| | 1BBL trimer | | IL21 fusion | | | | | |
| | fusion | | | | | | | |
| AVC-011 | Anti-NKp46 | SEQ ID | Anti-NKp46 | SEQ ID | Anti-NKp46 | SEQ ID | Anti-NKp46 | SEQ ID |
| | HC-4-1BBL | NO: 15 | HC-4-1BBL | NO: 15 | LC-IL21 | NO: 21 | LC-IL21 | NO: 21 |
| | trimer fusion | | trimer fusion | | fusion | | fusion | |
| AVC-012 | Anti-NKp46 | SEQ ID | Anti-NKp46 | SEQ ID | Anti-NKp46 | SEQ ID | Anti-NKp46 | SEQ ID |
| | CD16⁻ HC-4- | NO: 39 | CD16⁻ HC-4- | NO: 39 | LC-IL21 | NO: 21 | LC-IL21 | NO: 21 |
| | 1BBL trimer | | 1BBL trimer | | fusion | | fusion | |
| | fusion | | fusion | | | | | |
| AVC-013 | Anti-NKp46 | SEQ ID | Anti-NKp46 | SEQ ID | Anti-NKp46 | SEQ ID | Anti-NKp46 | SEQ ID |
| | HC-IL21 | NO: 18 | HC-IL21 | NO: 18 | LC | NO: 2 | LC | NO: 2 |
| | fusion | | fusion | | | | | |
| AVC-014 | IL21-Fc-region - | SEQ ID | IL21-Fc-region - | SEQ ID | — | — | — | — |
| | 4-1BBL trimer | NO: 19 | 4-1BBL trimer | NO: 19 | | | | |
| | fusion | | fusion | | | | | |
| AVC-015 | IL21- CD16⁻ | SEQ ID | IL21- CD16⁻ | SEQ ID | — | — | — | — |
| | Fc-region -4- | NO: 40 | Fc-region -4- | NO: 40 | | | | |
| | 1BBL trimer | | 1BBL trimer | | | | | |
| | fusion | | fusion | | | | | |
| AVC-006 | Anti-NKG2D | SEQ ID | Trastuzumab- | SEQ ID | Anti-NKG2D | SEQ ID | — | — |
| | HC1 | NO: 16 | scFv-HC2 | NO: 17 | LC | NO: 20 | | |

TABLE 1.1.2
Annotated amino acid sequences of multispecific binding molecules AVC-009-AVC-015.
Heavy chain 1 of AVC-009 (SEQ ID NO: 11) compared with VH3/3-11 (SEQ ID NO: 22) and
a human IgG1 constant region (SEQ ID NO: 23)

| | CDR-H1 CDR-H2 | |
|---|---|---|
| AVC-009 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-009 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-009 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-009 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-009 | EYKCKVSNKA LPAPIEKTIS KAKGQPREPQ VYTLPPCREE MTKNQVSLWC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-009 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-009 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-009 | HLQPLRSAAG AAALALTVDL PPASSEARNS AFGFQGRLLH LSAGQRLGVH LHTEARARHA WQLTQGATVL GLFRVTPEIP | 640 |
| | Linker | |
| AVC-009 | AGLPSPRSEG GGGSGGGGSG GGGSGGGGSR EGPELSPDDP AGLLDLRQGM FAQLVAQNVL LIDGPLSWYS DPGLAGVSLT | 720 |
| | 4-1BBL ECD | |
| AVC-009 | GGLSYKEDTK ELVVAKAGVY YVFFQLELRR VVAGEGSGSV SLALHLQPLR SAAGAAALAL TVDLPPASSE ARNSAFGFQG | 800 |
| | Linker | |
| AVC-009 | GLLHLSAGQR LGVHLHTEAR ARHAWQLTQG ATVLGLFRVT PEIPAGLPSP RSEGGGGSGG GGSGGGGSGG GGSREGPELS | 880 |
| | 4-1BBL ECD | |
| AVC-009 | PDDPAGLLDL RQGMFAQLVA QNVLLIDGPL SWYSDPGLAG VSLTGGLSYK EDTKELVVAK AGVYYVFFQL ELRRVVAGEG | 960 |
| AVC-009 | SGSVSLALHL QPLRSAAGAA ALALTVDLPP ASSEARNSAF GFQGRLLHLS AGQRLGVHLH TEARARHAWQ LTQGATVLGL | 1040 |
| AVC-009 | FRVTPEIPAG LPSPRSE | 1057 |

Heavy chain 2 of AVC-009 (SEQ ID NO: 12) compared with VH3/3-11 (SEQ ID NO: 22) and
a human IgG1 constant region (SEQ ID NO: 23)

| | | |
|---|---|---|
| | CDR-H1 CDR-H2 | |
| AVC-009 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-009 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-009 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-009 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-009 | EYKCKVSNKA LPAPIEKTIS KAKGQPREPQ VCTLPPSREE MTKNQVSLSC AVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Knobs-Into-Hole Linker | |
| AVC-009 | LDSDGSFFLV SKLTVDKSRW QQGNVFSCSV MHEALHNHYT QKSLSLSPGK GGGGSQGQDR HMIRMRQLID IVDQLKNYVN | 480 |
| HuIgG1 | LDSDGSFFLY SKLTVDKSRW QQGNVFSCSV MHEALHNHYT QKSLSLSPGK | |
| | IL-21 | |
| AVC-009 | DLVPEFLPAP EDVETNCEWS AFSCFQKAQL KSANTGNNER IINVSIKKLK RKPPSTNAGR RQKHRLTCPS CDSYEKKPPK | 560 |
| AVC-009 | EFLERFKSLL QKMIHQHLSS RTHGSEDS | 588 |

Heavy chain 1 of AVC-010 (SEQ ID NO: 13) compared with VH3/3-11 (SEQ ID NO: 22) and
a human IgG1 constant region (SEQ ID NO: 23)

| | | |
|---|---|---|
| | CDR-H1 CDR-H2 | |
| AVC-010 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-010 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge LALA | |
| AVC-010 | WNSGALTSGV HTFPAVLQSS GLYSLSSVVT VPSSSLGTQT YICNVNHKPS NTKVDKKVEP KSCDKTHTCP PCPAPEAAGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-010 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-010 | EYKCKVSNKA LPAPIEKTIS KAKGQPREPQ VYTLPPCREE MTKNQVSLWC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-010 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-010 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-010 | HLQPLRSAAG AAALALTVDL PPASSEARNS AFGFQGRLLH LSAGQRLGVH LHTEARARHA WQLTQGATVL GLFRVTPEIP | 640 |
| | Linker | |
| AVC-010 | AGLPSPRSEG GGGSGGGGSG GGGSGGGGSR EGPELSPDDP AGLLDLRQGM FAQLVAQNVL LIDGPLSWYS DPGLAGVSLT | 720 |
| | 4-1BBL ECD | |
| AVC-010 | GGLSYKEDTK ELVVAKAGVY YVFFQLELRR VVAGEGSGSV SLALHLQPLR SAAGAAALAL TVDLPPASSE ARNSAFGFQG | 800 |
| | Linker | |
| AVC-010 | GLLHLSAGQR LGVHLHTEAR ARHAWQLTQG ATVLGLFRVT PEIPAGLPSP RSEGGGGSGG GGSGGGGSGG GGSREGPELS | 880 |
| | 4-1BBL ECD | |
| AVC-010 | PDDPAGLLDL RQGMFAQLVA QNVLLIDGPL SWYSDPGLAG VSLTGGLSYK EDTKELVVAK AGVYYVFFQL ELRRVVAGEG | 960 |
| AVC-010 | SGSVSLALHL QPLRSAAGAA ALALTVDLPP ASSEARNSAF GFQGRLLHLS AGQRLGVHLH TEARARHAWQ LTQGATVLGL | 1040 |
| AVC-010 | FRVTPEIPAG LPSPRSE | 1057 |

Heavy chain 2 of AVC-010 (SEQ ID NO: 14) compared with VH3/3-11 (SEQ ID NO: 22) and
a human IgG1 constant region (SEQ ID NO: 23)

| | | |
|---|---|---|
| | CDR-H1 CDR-H2 | |
| AVC-010 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-010 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge LALA | |
| AVC-010 | WNSGALTSGV HTFPAVLQSS GLYSLSSVVT VPSSSLGTQT YICNVNHKPS NTKVDKKVEP KSCDKTHTCP PCPAPEAAGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-010 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ Knobs-Into-Hole | |
| AVC-010 | EYKCKVSNKA LPAPIEKTIS KAKGQPREPQ VCTLPPSREE MTKNQVSLSC AVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Knobs-Into-Hole Linker | |
| AVC-010 | LDSDGSFFLV SKLTVDKSRW QQGNVFSCSV MHEALHNHYT QKSLSLSPGK GGGGSQGQDR HMIRMRQLID IVDQLKNYVN | 480 |
| HuIgG1 | LDSDGSFFLY SKLTVDKSRW QQGNVFSCSV MHEALHNHYT QKSLSLSPGK | |
| | IL-21 | |
| AVC-010 | DLVPEFLPAP EDVETNCEWS AFSCFQKAQL KSANTGNNER IINVSIKKLK RKPPSTNAGR RQKHRLTCPS CDSYEKKPPK | 560 |
| AVC-010 | EFLERFKSLL QKMIHQHLSS RTHGSEDS | 588 |

Heavy chain of AVC-011 (SEQ ID NO: 15) compared with VH3/3-11 (SEQ ID NO: 22) and
a human IgG1 constant region (SEQ ID NO: 23)

| | | |
|---|---|---|
| | CDR-H1 CDR-H2 | |
| AVC-011 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-011 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-011 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-011 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ | |
| AVC-011 | EYKCKVSNKA LPAPIEKTIS KAKGQPREPQ VYTLPPSREE MTKNQVSLTC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-011 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-011 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-011 | HLQPLRSAAG AAALALTVDL PPASSEARNS AFGFQGRLLH LSAGQRLGVH LHTEARARHA WQLTQGATVL GLFRVTPEIP | 640 |
| | Linker | |
| AVC-011 | AGLPSPRSEG GGGSGGGGSG GGGSGGGGSR EGPELSPDDP AGLLDLRQGM FAQLVAQNVL LIDGPLSWYS DPGLAGVSLT | 720 |
| | 4-1BBL ECD | |
| AVC-011 | GGLSYKEDTK ELVVAKAGVY YVFFQLELRR VVAGEGSGSV SLALHLQPLR SAAGAAALAL TVDLPPASSE ARNSAFGFQG | 800 |
| | Linker | |
| AVC-011 | GLLHLSAGQR LGVHLHTEAR ARHAWQLTQG ATVLGLFRVT PEIPAGLPSP RSEGGGGSGG GGSGGGGSGG GGSREGPELS | 880 |
| | 4-1BBL ECD | |
| AVC-011 | PDDPAGLLDL RQGMFAQLVA QNVLLIDGPL SWYSDPGLAG VSLTGGLSYK EDTKELVVAK AGVYYVFFQL ELRRVVAGEG | 960 |
| AVC-011 | SGSVSLALHL QPLRSAAGAA ALALTVDLPP ASSEARNSAF GFQGRLLHLS AGQRLGVHLH TEARARHAWQ LTQGATVLGL | 1040 |
| AVC-011 | FRVTPEIPAG LPSPRSE | 1057 |

Light chain of AVC-011 and AVC-012 (SEQ ID NO: 21) compared with VK3/A27 (SEQ ID NO: 49)

| | | |
|---|---|---|
| | CDR-L1 CDR-L2 | |
| AVC-011 | DIQMTQTTSS LSASLGDRVT ISCRASQDIS -NYLNWYQQKP DGTVKLLIYY TSRLHSGVPS RFSGSGSGTD YSLTINNLEQ | 80 |
| VK3/A27 | EIVLTQSPGT LSLSPGERAT LSCRASQSVS SSYLAWYQQKP GQAPRLLIYG ASSRATGIPD RFSGSGSGTD FTLTISRLEP | |
| | CDR-L3 CL→ | |
| AVC-011 | EDIATYFCQQ GNTRPWTFGG GTKLEIKRTV AAPSVFIFPP SDEQLKSGTA SVVCLLNNFY PREAKVQWKV DNALQSGNSQ | 160 |
| VK3/A27 | EDFAVYYCQQ YGSSP | |
| | Linker | |
| AVC-011 | ESVTEQDSKD STYSLSSTLT LSKADYEKHK VYACEVTHQG LSSPVTKSFN RGECGGGGSQ GQDRHMIRMR QLIDIVDQLK | 240 |
| | IL-21 | |
| AVC-011 | NYVNDLVPEF LPAPEDVETN CEWSAFSCFQ KAQLKSANTG NNERIINVSI KKLKRKPPST NAGRRQKHRL TCPSCDSYEK | 320 |
| AVC-011 | KPPKEFLERF KSLLQKMIHQ HLSSRTHGSE DS | 352 |

Heavy chain of AVC-012 (SEQ ID NO: 39) compared with VH3/3-11 (SEQ ID NO: 22) and
a human IgG1 constant region (SEQ ID NO: 23)

| | | |
|---|---|---|
| | CDR-H1 CDR-H2 | |
| AVC-012 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-012 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge LALA | |
| AVC-012 | WNSGALTSGV HTFPAVLQSS GLYSLSSVVT VPSSSLGTQT YICNVNHKPS NTKVDKKVEP KSCDKTHTCP PCPAPEAAGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-012 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ | |
| AVC-012 | EYKCKVSNKA LPAPIEKTIS KAKGQPREPQ VYTLPPSREE MTKNQVSLTC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-012 | LDSDGSFFLY SKLTVDKSRW QQGNVFSCSV MHEALHNHYT QKSLSLSPGK GGGGSGGGGS GGGGSREGPE LSPDDPAGLL | 480 |
| HuIgG1 | LDSDGSFFLY SKLTVDKSRW QQGNVFSCSV MHEALHNHYT QKSLSLSPGK | |
| AVC-012 | DLRQGMFAQL VAQNVLLIDG PLSWYSDPGL AGVSLTGGLS YKEDTKELVV AKAGVYYVFF QLELRRVVAG EGSGSVSLAL | 560 |
| | 4-1BBL ECD | |
| AVC-012 | HLQPLRSAAG AAALALTVDL PPASSEARNS AFGFQGRLLH LSAGQRLGVH LHTEARARHA WQLTQGATVL GLFRVTPEIP | 640 |
| | Linker | |
| AVC-012 | AGLPSPRSEG GGGSGGGGSG GGGSGGGGSR EGPELSPDDP AGLLDLRQGM FAQLVAQNVL LIDGPLSWYS DPGLAGVSLT | 720 |
| | 4-1BBL ECD | |
| AVC-012 | GGLSYKEDTK ELVVAKAGVY YVFFQLELRR VVAGEGSGSV SLALHLQPLR SAAGAAALAL TVDLPPASSE ARNSAFGFQG | 800 |
| | Linker | |
| AVC-012 | GLLHLSAGQR LGVHLHTEAR ARHAWQLTQG ATVLGLFRVT PEIPAGLPSP RSEGGGGSGG GGSGGGGSGG GGSREGPELS | 880 |
| | 4-1BBL ECD | |
| AVC-012 | PDDPAGLLDL RQGMFAQLVA QNVLLIDGPL SWYSDPGLAG VSLTGGLSYK EDTKELVVAK AGVYYVFFQL ELRRVVAGEG | 960 |
| AVC-012 | SGSVSLALHL QPLRSAAGAA ALALTVDLPP ASSEARNSAF GFQGRLLHLS AGQRLGVHLH TEARARHAWQ LTQGATVLGL | 1040 |
| AVC-012 | FRVTPEIPAG LPSPRSE | 1057 |

Heavy chain of AVC-013 (SEQ ID NO: 18) compared with VH3/3-11 (SEQ ID NO: 22) and
a human IgG1 constant region (SEQ ID NO: 23)

| | | |
|---|---|---|
| | CDR-H1 CDR-H2 | |
| AVC-013 | QVQLQQSGPE LVKPGASVKM SCKASGYTFT DYVINWGKQR SGQGLEWIGE IYPGSGTNYY NEKFKAKATL TADKSSNIAY | 80 |
| VH3/3-11 | QVQLVESGGG LVKPGGSLRL SCAASGFTFS DYYMSWIRQA PGKGLEWVSY ISSSGSTIYY ADSVKGRFTI SRDNAKNSLY | |
| | CDR-H3 CH1→ | |
| AVC-013 | MQLSSLTSED SAVYFCARRG RYGLYAMDYW GQGTSVTVSS ASTKGPSVFP LAPSSKSTSG GTAALGCLVK DYFPEPVTVS | 160 |
| VH3/3-11 | LQMNSLRAED TAVYYCAR ASTKGPSVFP LAPSSKSTSG GTAALGCLVK DYFPEPVTVS | |
| | Hinge | |
| AVC-013 | WNSGALTSGV HTFPAVLQSS GLYSLSSVVT VPSSSLGTQT YICNVNHKPS NTKVDKKVEP KSCDKTHTCP PCPAPELLGG | 240 |
| HuIgG1 | WNSGALTSGV HTFPAVLQSS GLYSLSSVVT VPSSSLGTQT YICNVNHKPS NTKVDKKVEP KSCDKTHTCP PCPAPELLGG | |
| | CH2→ | |
| AVC-013 | PSVFLFPPKP KDTLMISRTP EVTCVVVDVS HEDPEVKFNW YVDGVEVHNA KTKPREEQYN STYRVVSVLT VLHQDWLNGK | 320 |
| HuIgG1 | PSVFLFPPKP KDTLMISRTP EVTCVVVDVS HEDPEVKFNW YVDGVEVHNA KTKPREEQYN STYRVVSVLT VLHQDWLNGK | |
| | CH3→ | |
| AVC-013 | EYKCKVSNKA LPAPIEKTIS KAKGQPREPQ VYTLPPSREE MTKNQVSLTC LVKGFYPSDI AVEWESNGQP ENNYKTTPPV | 400 |
| HuIgG1 | EYKCKVSNKA LPAPIEKTIS KAKGQPREPQ VYTLPPSRDE LTKNQVSLTC LVKGFYPSDI AVEWESNGQP ENNYKTTPPV | |
| | Linker | |
| AVC-013 | LDSDGSFFLY SKLTVDKSRW QQGNVFSCSV MHEALHNHYT QKSLSLSPGK GGGGSQGQDR HMIRMRQLID IVDQLKNYVN | 480 |
| HuIgG1 | LDSDGSFFLY SKLTVDKSRW QQGNVFSCSV MHEALHNHYT QKSLSLSPGK | |
| | IL-21 | |
| AVC-013 | DLVPEFLPAP EDVETNCEWS AFSCFQKAQL KSANTGNNER IINVSIKKLK RKPPSTNAGR RQKHRLTCPS CDSYEKKPPK | 560 |
| AVC-013 | EFLERFKSLL QKMIHQHLSS RTHGSEDS | 588 |

Light chain of AVC-009, AVC-010 and AVC-013 (SEQ ID NO: 2)
compared with VK3/A27 (SEQ ID NO: 49)

| | | |
|---|---|---|
| | CDR-L1 CDR-L2 | |
| AVC-009 | DIQMTQTTSS LSASLGDRVT ISCRASQDIS -NYLNWYQQKP DGTVKLLIYY TSRLHSGVPS RESGSGSGTD YSLTINNLEQ | 80 |
| VK3/A27 | EIVLTQSPGT LSLSPGERAT LSCRASQSVS SSYLAWYQQKP GQAPRLLIYG ASSRATGIPD RFSGSGSGTD FTLTISRLEP | |
| | CDR-L3 CL→ | |
| AVC-009 | EDIATYFCQQ GNTRPWTFGG GTKLEIKRTV AAPSVFIFPP SDEQLKSGTA SVVCLLNNFY PREAKVQWKV DNALQSGNSQ | 160 |
| VK3/A27 | EDFAVYYCQQ YGSSP | |
| AVC-009 | ESVTEQDSKD STYSLSSTLT LSKADYEKHK VYACEVTHQG LSSPVTKSFN RGEC | 214 |

Heavy chain of AVC-014 (SEQ ID NO: 19) compared
with a human IgG1 constant region (SEQ ID NO: 23)

| | | |
|---|---|---|
| | IL-21 | |
| AVC-014 | QGQDRHMIRM RQLIDIVDQL KNYVNDLVPE FLPAPEDVET NCEWSAFSCF QKAQLKSANT GNNERIINVS IKKLKRKPPS | 80 |
| | Hinge CH2→ | |
| AVC-014 | TNAGRRQKHR LTCPSCDSYE KKPPKEFLER FKSLLQKMIH QHLSSRTHGS EDSVEPKSCD KTHTCPPCPA PELLGGPSVF | 160 |
| HuIgG1 | VEPKSCD KTHTCPPCPA PELLGGPSVF | |
| AVC-014 | LFPPKPKDTL MISRTPEVTC VVVDVSHEDP EVKFNWYVDG VEVHNAKTKP REEQYNSTYR VVSVLTVLHQ DWLNGKEYKC | 240 |
| HuIgG1 | LFPPKPKDTL MISRTPEVTC VVVDVSHEDP EVKFNWYVDG VEVHNAKTKP REEQYNSTYR VVSVLTVLHQ DWLNGKEYKC | |
| | CH3→ | |
| AVC-014 | KVSNKALPAP IEKTISKAKG QPREPQVYTL PPSREEMTKN QVSLTCLVKG FYPSDIAVEW ESNGQPENNY KTTPPVLDSD | 320 |
| HuIgG1 | KVSNKALPAP IEKTISKAKG QPREPQVYTL PPSRDELTKN QVSLTCLVKG FYPSDIAVEW ESNGQPENNY KTTPPVLDSD | |
| | Linker | |
| AVC-014 | GSFFLYSKLT VDKSRWQQGN VFSCSVMHEA LHNHYTQKSL SLSPGKGGGG SGGGGSGGGG SREGPELSPD DPAGLLDLRQ | 400 |
| HuIgG1 | GSFFLYSKLT VDKSRWQQGN VFSCSVMHEA LHNHYTQKSL SLSPGK | |
| AVC-014 | GMFAQLVAQN VLLIDGPLSW YSDPGLAGVS LTGGLSYKED TKELVVAKAG VYYVFFQLEL RRVVAGEGSG SVSLALHLQP | 480 |
| | 4-1BBL ECD | |
| AVC-014 | LRSAAGAAAL ALTVDLPPAS SEARNSAFGF QGRLLHLSAG QRLGVHLHTE ARARHAWQLT QGATVLGLFR VTPEIPAGLP | 560 |
| | Linker | |
| AVC-014 | SPRSEGGGGS GGGGSGGGGS GGGGSREGPE LSPDDPAGLL DLRQGMFAQL VAQNVLLIDG PLSWYSDPGL AGVSLTGGLS | 640 |
| | 4-1BBL ECD | |
| AVC-014 | YKEDTKELVV AKAGVYYVFF QLELRRVVAG EGSGSVSLAL HLQPLRSAAG AAALALTVDL PPASSEARNS AFGFQGGLLH | 720 |
| | Linker | |
| AVC-014 | LSAGQRLGVH LHTEARARHA WQLTQGATVL GLFRVTPEIP AGLPSPRSEG GGGSGGGGSG GGGSGGGGSR EGPELSPDDP | 800 |
| | 4-1BBL ECD | |
| AVC-014 | AGLLDLRQGM FAQLVAQNVL LIDGPLSWYS DPGLAGVSLT GGLSYKEDTK ELVVAKAGVY YVFFQLELRR VVAGEGSGSV | 880 |
| AVC-014 | SLALHLQPLR SAAGAAALAL TVDLPPASSE ARNSAFGFQG RLLHLSAGQR LGVHLHTEAR ARHAWQLTQG ATVLGLFRVT | 960 |
| AVC-014 | PEIPAGLPSP RSE | 973 |

Heavy chain of AVC-015 (SEQ ID NO: 40) compared with
a human IgG1 constant region (SEQ ID NO: 23)

| | | |
|---|---|---|
| | IL-21 | |
| AVC-014 | QGQDRHMIRM RQLIDIVDQL KNYVNDLVPE FLPAPEDVET NCEWSAFSCF QKAQLKSANT GNNERIINVS IKKLKRKPPS | 80 |
| | Hinge LALA CH2→ | |
| AVC-014 | TNAGRRQKHR LTCPSCDSYE KKPPKEFLER FKSLLQKMIH QHLSSRTHGS EDSVEPKSCD KTHTCPPCPA PEAAGGPSVF | 160 |
| HuIgG1 | VEPKSCD KTHTCPPCPA PELLGGPSVF | |
| AVC-014 | LFPPKPKDTL MISRTPEVTC VVVDVSHEDP EVKFNWYVDG VEVHNAKTKP REEQYNSTYR VVSVLTVLHQ DWLNGKEYKC | 240 |
| HuIgG1 | LFPPKPKDTL MISRTPEVTC VVVDVSHEDP EVKFNWYVDG VEVHNAKTKP REEQYNSTYR VVSVLTVLHQ DWLNGKEYKC | |
| | CH3→ | |
| AVC-014 | KVSNKALPAP IEKTISKAKG QPREPQVYTL PPSREEMTKN QVSLTCLVKG FYPSDIAVEW ESNGQPENNY KTTPPVLDSD | 320 |
| HuIgG1 | KVSNKALPAP IEKTISKAKG QPREPQVYTL PPSRDELTKN QVSLTCLVKG FYPSDIAVEW ESNGQPENNY KTTPPVLDSD | |
| | Linker | |
| AVC-014 | GSFFLYSKLT VDKSRWQQGN VFSCSVMHEA LHNHYTQKSL SLSPGKGGGG SGGGGSGGGG SREGPELSPD DPAGLLDLRQ | 400 |
| HuIgG1 | GSFFLYSKLT VDKSRWQQGN VFSCSVMHEA LHNHYTQKSL SLSPGK | |
| AVC-014 | GMFAQLVAQN VLLIDGPLSW YSDPGLAGVS LTGGLSYKED TKELVVAKAG VYYVFFQLEL RRVVAGEGSG SVSLALHLQP | 480 |
| | 4-1BBL ECD | |
| AVC-014 | LRSAAGAAAL ALTVDLPPAS SEARNSAFGF QGRLLHLSAG QRLGVHLHTE ARARHAWQLT QGATVLGLFR VTPEIPAGLP | 560 |
| | Linker | |
| AVC-014 | SPRSEGGGGS GGGGSGGGGS GGGGSREGPE LSPDDPAGLL DLRQGMFAQL VAQNVLLIDG PLSWYSDPGL AGVSLTGGLS | 640 |
| | 4-1BBL ECD | |
| AVC-014 | YKEDTKELVV AKAGVYYVFF QLELRRVVAG EGSGSVSLAL HLQPLRSAAG AAALALTVDL PPASSEARNS AFGFQGGLLH | 720 |
| | Linker | |
| AVC-014 | LSAGQRLGVH LHTEARARHA WQLTQGATVL GLFRVTPEIP AGLPSPRSEG GGGSGGGGSG GGGSGGGGSR EGPELSPDDP | 800 |
| | 4-1BBL ECD | |
| AVC-014 | AGLLDLRQGM FAQLVAQNVL LIDGPLSWYS DPGLAGVSLT GGLSYKEDTK ELVVAKAGVY YVFFQLELRR VVAGEGSGSV | 880 |
| AVC-014 | SLALHLQPLR SAAGAAALAL TVDLPPASSE ARNSAFGFQG RLLHLSAGQR LGVHLHTEAR ARHAWQLTQG ATVLGLFRVT | 960 |
| AVC-014 | PEIPAGLPSP RSE | 973 |

CDR and hinge sequences are indicated and underlined. Constant regions are indicated with arrows. Linker sequences are indicated and underlined and in italics. Knobs-into-hole modifications are indicated and underlined, bold and in italics. L234A/L235A modifications in the Fc region for reduced binding to CD16 are indicated (“LALA”) and underlined in bold. Receptor agonists are indicated.

### Example 2 Biochemical Characterization of Multispecific Antigen Binding Proteins

### 2.1. Expression Level and Molecular Weight

Expression levels and molecular weight of the multispecific antigen binding proteins is determined by SDS-PAGE and Size Exclusion Chromatography (SEC). Coomassie blue-stained 4% to 15% SDS-PAGE is performed under both reducing and non-reducing conditions (Zhang et al. Clin Cancer Res, 2007; 13 (9): 2758-2767).

Size exclusion chromatography is performed using a Yarra SEC-3000 column (Phenomenex, 00H-4513-K0) and a Waters 2695 HPLC (Waters Corporation) in a 0.1 M Na₂HPO₄/NaH₂PO₄, 0.1 M Na₂SO₄, pH 6.7 mobile phase at a flow rate of 0.5 mL/min. Thyroglobulin (669 kDa, Sr 8.5 nm), b-amylase (200 kDa, Sr 5.4 nm), bovine serum albumin (67 kDa, Sr 3.55 nm), carbonic anhydrase (29 kDa, Sr 2.35 nm) and FLAG peptide (1 kDa) served as standard proteins.

### 2.2 Stability

Stability of the multispecific antigen binding proteins is determined by repeating the above SDS-PAGE and SEC on samples of the multispecific antigen binding proteins that are stored at room temperature for for 1, 3, and 7 days.

Stability of the multispecific antigen binding proteins in plasma is determined by incubating the proteins at 200 nM in 50% human plasma at 37° C. for 1, 3, and 7 days.

Samples are frozen at −20° C. immediately after preparation (0 d) or after the respective incubation period. The level of intact protein is determined using SDS-PAGE and SEC as described above.

### 2.3 Affinity for IL21R

Binding affinity between IL-21R and multispecific antigen binding proteins was analyzed using a Biacore T200 instrument at 25° C. and a flow rate of 50 μl/min. Anti-human Fc antibody was covalently immobilized onto a CM5 sensor chip to capture the multispecific antigen binding proteins in flow cells fc2 and fc4, with fc1 and fc3 as references. IL-21R was introduced at concentrations of 0.74 nM, 2.22 nM, 6.67 nM, 20 nM, and 60 nM, prepared through a series of 1:3 serial dilutions. The assay included a 180-second association phase and a 1200-second dissociation phase after the highest concentration exposure. The analysis buffer contained 10 mM HEPES (pH 7.4), 150 mM NaCl, 0.05% Tween 20, and 3 mM EDTA. Regeneration was performed under standard conditions to remove the complex from all surfaces

### 2.4 Affinity for 4-1BB

Interaction between the multispecific antigen binding proteins and the 4-1BB receptor was analyzed utilizing a Biacore T200 instrument at 25° C. with a 50 μl/min flow rate. The CM5 sensor chip from the Human Antibody Capture Kit (Cytiva) was used, and the analysis buffer was composed of 10 mM HEPES (pH 7.4), 150 mM NaCl, 0.05% Tween 20, and 3 mM EDTA. Anti-human Fc antibody was covalently immobilized onto the CM5 sensor chip to capture the multispecific antigen binding proteins on flow cells fc2 and fc4, while fc1 and fc3 served as references. Human 4-1BB was introduced in concentrations of 5 nM, 15 nM, 45 nM, 135 nM, and 405 nM, following a series of 1:3 serial dilutions. The assay protocol included a 120-second association phase and a 600-second dissociation phase post the introduction of the highest concentration sample. Following the interaction analyses, regeneration was conducted under standard conditions to effectively remove the complex from all surfaces. Recombinant biotinylated human, cynomolgus and murine 4-1BB Fc (kih) fusion molecules (see Example 3 of WO 2016/075278) are directly coupled on a SA chip using the standard coupling instruction (Biacore, Freiburg/Germany). The immobilization level is about 30 RU. The multispecific antigen binding or controls, are passed at a concentration range from 0.39 to 200 nM with a flow of 30 μL/minute through the flow cells over 120 seconds. The dissociation is monitored for 180 seconds. Bulk refractive index differences are corrected for by subtracting the response obtained on a reference empty flow cell.

For affinity measurement, direct coupling of around 7200 resonance units (RU) of an anti-human Fc specific antibody is performed on a CM5 chip at pH 5.0 using the standard amine coupling kit (GF Healthcare). 4-1BBL-containing multispecific antigen binding proteins or controls, at 50 nM are captured with a flow rate of 30 μL/minute for 60 seconds on flow cell 2. A dilution series (1.95 to 1000 nM) of human 4-1BB-avi-His (see Example 3 of WO 2016/075278) is passed on both flow cells at 30 μL/minute for 180 seconds to record the association phase. The dissociation phase is monitored for 180 s and triggered by switching from the sample solution to HBS-EP. The chip surface is regenerated after every cycle using a double injection of 60 sec 10 mM Glycine-HCl pH 2.1. Bulk refractive index differences are corrected for by subtracting the response obtained on reference flow cell 1. For the interaction between the 4-1BBL-containing multispecific antigen binding proteins and hu4-IBB avi His, the affinity constants are derived from the rate constants by fitting to a 1:1 Langmuir binding curve using the Biaeval software (GF Healthcare).

### 2.5 Affinity for NKp46

Affinity of the multispecific antigen binding proteins for NKp46 was determined by SPR, essentially as described in Gauthier et al., 2019, Cell 177, 1701-1713.

### Example 3 Functional In Vitro Characterization of Multispecific Antigen Binding Proteins

### 3.1 Binding to NK Cells

Binding of the multispecific antigen binding proteins to NK cells is demonstrated using flow cytometry and competitive inhibition with unlabeled competing antibodies, essentially as described by Fellermeier et al. (2016, supra). Assays are performed in triplicate against NK cells from four different donors. Mean fluorescent intensity is plotted against dilutions of multispecific antigen binding proteins to determine EC50 and EC90 concentrations for optimal effective engaging.

### 3.2 Short Term NK Cell Cytotoxicity

Short term (4-hour) NK cell cytotoxicity of the multispecific antigen binding proteins was determined using the calceine-acetoxymethyl-release assay (Somanchi et al., 2011, J Vis Exp., 2:2540; Lee et al., 2010, Methods Mol. Biol., 651:61-77). Target cells (MDA-MB-231, K562, Karpas 299, SK-N-AS and A253) were loaded with 0.025 μM Calcein-AM and incubated for 1 hour at 37° C. with gentle resuspension every 10-15 minutes. After this target cells were washed twice with medium and 10,000 target cells were seeded in a 96-well plate. NK cells were purified via negative selection using RosetteSep (In: Methods in Molecular Biology, Ex Vivo Expansion of Human NK Cells Using K562 Engineered to Express Membrane Bound IL21 Srinivas S. Somanchi and Dean A. Lee DOI 10.1007/978-1-4939-3684-7) from four normal donor buffy coats and expanded ex vivo with (saturating concentration) of the multispecific antigen binding proteins AVC-009 AVC-011 or AVC-014 as described in Example 3.5 below. As control, NK cells were stimulated with irradiated FC21 feeder cells at a ratio of 1:2 (NK:FC21). Assays were performed in triplicate. Cytotoxicity of NK cells expanded ex vivo for 1 week in the presence of a multispecific antigen binding protein (AVC-009 AVC-011 or AVC-014), was compared to that of the NK cells expanded ex vivo in the presence of irradiated FC21 feeder cells. NK cells were taken from the expansions and added in an E:T ration of 2:1 to the target cells, and co-cultured for 4 hours at 37° C. In addition two control conditions are setup as well:
1. Target cells only—this sample was used to quantify the spontaneous release of Calcein-AM from the tumor cells
2. Maximum release—this sample was treated with Triton X-100 to kill and permeabilize the target cells and used to quantify the maximum possible release of Calcein-AM
   After this incubation supernatant was transferred to a new plate and fluorescence was measured on a plate reader using an excitation filter at 485 nm and an emission filter at 530 nm. Specific lysis was calculated using the following formula:

$$\% Specic Lysis = \frac{( Fluores {ence}_{Experimental} - {Fluorescence}_{Spontaneous} )}{( {Fluorescence}_{Maximum} - {Fluorescence}_{Spontaneous} )} \times 100$$

The results are shown in FIG. 3 and demonstrate that all three multispecific antigen binding proteins AVC-009, AVC-011 and AVC-014 induce strong cytotoxicity of NK cells against MDA-MB-231 target cells. Moreover, the cytotoxicity of NK cells expanded in the presence of AVC-009 or AVC-014 does not even significantly differ from that of NK cell stimulated by FC21 feeder cells. Similar data were obtained with K562, Karpas 299, SK-N-AS and A253 as target cells (data not shown).

### 3.3 Long-Term NK Cell Cytotoxicity

Long-term NK cell cytotoxicity of the multispecific antigen binding proteins, to assess serial killing, is determined using the xCelligence assay (Naeimi Kararoudi et al., 2022, Cell Reports Methods 2, 100236 Jun. 20, 2022). Hematological target cells are K562 and Karpas 299 (T cell NHL). Solid tumor target cell lines are SK-N-AS (neuroblastoma) and A253 (salivary gland carcinoma). NK cells are purified via negative selection using RosetteSep (see above) from four normal donor buffy coats and co-cultured at low E:T ratio (0.5:1), with and without (saturating concentration) of the multispecific antigen binding proteins, or with the anti-NKp46 antibody of SEQ ID NO.'s: 1 and 2. Assays are performed in triplicate. Cytotoxicity of fresh NK cells (above) is compared with cytotoxicity of NK cells i) expanded ex vivo in the presence of a multispecific antigen binding protein, and ii) NK cells expanded ex vivo in the presence of with irradiated FC21 feeder cells. NK cells are expanded ex vivo as described in 3.5 below.

### 3.4 NK Cell Proliferation Assay

NK cells are labeled with CellTrace Violet Cell Proliferation Dye and expanded, with and without (saturating concentration or three dose levels, low, medium and high, e.g. 25, 50 and 100%) of the multispecific antigen binding proteins. Assays are performed in triplicate. After 7 days cells are harvested, stained for viability with Live/Dead stain and for surface markers to gate on the viable CD3-CD56+NK cell population. Proliferation is plotted against tumor cell and booster dilutions to identify EC50 and EC90 concentrations for optimal effective concentrations.

### 3.5 Ex Vivo Expansion of NK Cells

Protocols for ex vivo expansion of donor NK cells are described in Denman et al. (2012, supra).

Briefly, NK cells were isolated from 8 healthy donor buffy coats with RosetteSep enrichment and Ficoll (GE HealthCare, Piscataway, NJ). At day 0, NK cells were stimulated as follows: 1.0×10⁶ NK cells were incubated with 25 nM of each of the multispecific antigen binding proteins AVC-009, AVC-011 or AVC-014. As control, NK cells were stimulated with irradiated (100 cGy) FC21 feeder cells at a ratio of 1:2 (NK:FC21), in NK cell media at 1×10⁶ NK/mL. Cells were then plated at a density of 0.2×10⁶ cells/mL in AIM V™ media (12055091, Gibco, Thermo Scientific) supplemented with CTS™ immune cell serum replacement (A2596101, Gibco, Thermo Scientific) and with 50 IU/mL recombinant human IL-2 (Proleukin, Novartis Vaccines and Diagnostics, Inc). Every other day of expansion, NK Cells are counted using a Nexcelom Cellometer and AOPI staining solution. Total cell counts are determined. AIM V media supplemented with CTS immune cell serum replacement is added to keep the cells around a concentration of 0.3×10⁶ NK cells/ml of medium. 50 IU/mL human IL-2 is added to each condition/donor.

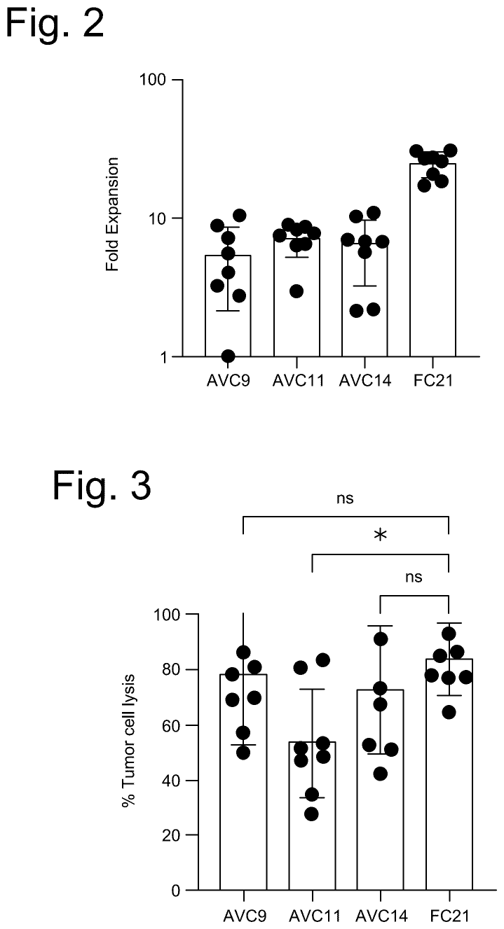

FIG. 2 shows that each of the multispecific antigen binding proteins AVC-009 or AVC-011, and AVC-014 as capable of inducing ex vivo expansion of NK cells. FC21 is the reference expansion method.

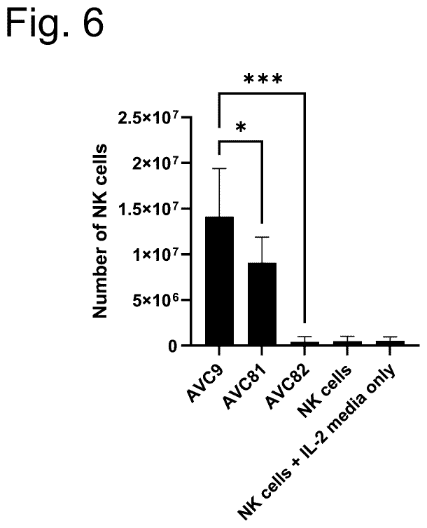

FIG. 6 shows represents a graph comparing the different rates of NK cell proliferation induced by AVC9 (IL-21 and 4-1BBL), AVC81 (IL-21) and AVC82 (4-1BBL). As can be seen, after 2 weeks of expansion cells were counted and expansion induced by AVC9 (IL-21 and 4-1BBL) is superior to both AVC81 (IL-21) and AVC82 (4-1BBL).

### Example 4 Phenotypic Characterization of NK Cells

### 4.1 Cytokine Secretion by Expanded NK Cells

NK cells are cultured for 7 days in the absence and presence of saturating concentrations of the multispecific antigen binding proteins. Supernatants are collected and concentrations of TNF-α, IFN-γ, IL-2 and IL-6 are simultaneously determined using microparticle-based cytokine capture on Cytometric Based Array kits. Concentrations were calculated from mean fluorescent intensity based on standard curves and formulas provided with the kit (Denman et al. PLOS ONE, 2012, supra). Cytokine secretion of fresh NK cells (above) is compared with cytokine secretion by NK cells i) expanded ex vivo in the presence of a multispecific antigen binding protein, and ii) NK cells expanded ex vivo in the presence of with irradiated FC21 feeder cells. NK cells are expanded ex vivo as described in 3.5 above.

### 4.2 Transcriptome Analysis of Expanded NK Cells

Gene expression in NK cells stimulated with the multispecific antigen binding proteins is assessed using the nCounter platform (nanoString Technologies, Inc., Seattle, WA; Geiss et al. 2008, Nat Biotechnol 26:317-325). Purified NK cells from four donors are stimulated for one week in parallel expansions with either a multispecific antigen binding protein or FC21 feeder cells (Denman et al. PLOS ONE, 2012, supra). Total RNA is purified from each sample and assessed for expression of 96 genes (Denman et al. PLOS ONE, 2012, supra). Gene expression is normalized to LDH (mean 6,076 copies detected from 100 ng of loaded mRNA), and plotted as mean±SEM. Genes with borderline detection ($10 normalized transcripts detected) are excluded from further analyses. Genes having >2-fold difference in mean expression between cultures with the multispecific antigen binding protein and FC21 are then identified.

### 4.3 Analysis of NK-Cell Phenotypes by Multiparameter Mass Cytometry

NK-cell phenotypes are identified by multiparameter mass cytometry. At the start of the expansion (baseline) and after one week of the expansion in the presence of the multispecific antigen binding proteins AVC-009, AVC-011 or AVC-014, or FC21 feeder cells (see Example 3.5), NK cells were collected and incubated with the antibodies listed in Table 4.3.1. Following this, cells were fixed in 2% formaldehyde in PBS and stored in this solution until acquisition.

TABLE 4.3.1
Antibodies conjugated to stable metal isotopes as used for mass cytometry. All the
antibodies were either purchased pre-conjugated from Fluidigm or were conjugated using X8
MaxPar conjugation kits according to the manufacture''s protocol.

| Antibody target | Metal tag | Antibody target | Metal tag |
|---|---|---|---|
| CD56 | 163Dy | TIGIT | 157Gd |
| CD16 | 207Pb | CXCR4 | 158Gd |
| CD57 | 176Yb | NKp30 | 159Tb |
| NKG2C | 142Ce | CXCR6 | 160Gd |
| CD137 | 145Nd | Ki67 | 161Dy |
| NKp44 | 146Nd | NKp46 | 162Dy |
| CD161 | 148Nd | Fas-L | 164Dy |
| CD244 | 149Sm | LAG3 | 165Ho |
| CD62L | 150Nd | NKG2D | 166Er |
| CD107a | 151Eu | NKG2A | 169Tm |
| KIR2DL1 | 152Sm | TIM3 | 170Er |
| HLA-DR | 153Eu | DNAM-1 | 171Yb |
| CD69 | 154Sm | NKp80 | 172Yb |
| CD27 | 155Gd | CD94 | 174Yb |
| CXCR3 | 156Gd | Perforin | 175Lu |

Immediately prior to acquisition, the samples were washed with Cell Staining Media (CSM; PBS+0.5% FBS), and resuspended in 1:20 dilution of EQT Four Element Calibration Beads (Fluidigm) at a concentration of 1 million cells/mL in CSM as previously described (Rahman et al., 2016, Cytometry A, 89:601-607).

After acquisition on a Helios mass cytometer (Fluidigm), the resulting FCS files were normalized using a normalization tool developed by Finck et al. and analyzed on Cyto-bank (www.cytobank.org) (Finck et al., 2013, Cytometry A, 83 (5): 483-94; Kotecha et al., Curr Protoc Cytom. 2010 July; Chapter 10: Unit10.17).

Once the singlet gate was established, cells were identified using markers (listed in Table 4.3.1) and analyzed using viSNE (a visualization tool for high-dimensional single-cell data based on the t-Distributed Stochastic Neighbor Embedding (t-SNE) algorithm) (see Amir et al., 2013, Nat. Biotechnol. 31 (6): 545-552).

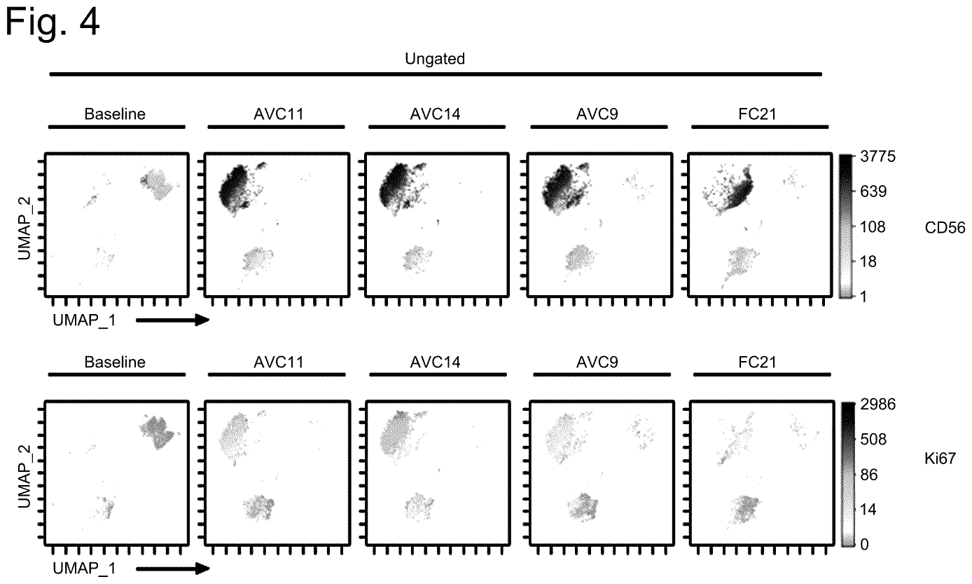

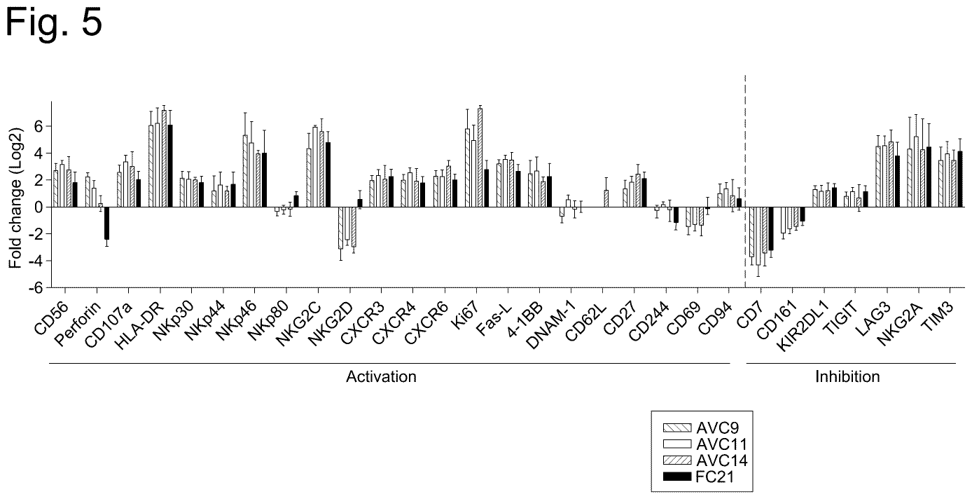

The results in FIGS. 4 and 5 show that the phenotype of NK cells stimulated and activated by AVC-009, AVC-011 or AVC-014 during expansion, closely matches the previously described hyper-functional phenotype of NK cells stimulated and activated by the reference FC21 feeder cells (Denman et al. (2012, supra). Main differences between the phenotypes induced by the multispecific antigen binding proteins and the FC21 feeder cells are increases in Ki67 and perforin expression (see also FIG. 5).

### Example 5 In Vivo Characterization of Multispecific Antigen Binding Proteins

In vivo PK and biodistribution of multispecific antigen binding proteins is performed using whole body clearance studies in BALB/c mice with radiolabeled fusion proteins as described in Zhang et al. (Clin Cancer Res, 2007, supra).

### EMBODIMENTS

1. A multispecific antigen binding protein comprising:
   a) an NK cell-activating cytokine that is at least one of:
      i) an interleukin 21 receptor (IL21R) agonist; and,
      ii) a 4-1BB agonist; and,
   b) a region that has affinity for a surface antigen expressed on natural killer (NK) cells.
2. A multispecific antigen binding protein according to embodiment 1, wherein the IL21R agonist comprises or consist of an IL21 polypeptide or an agonistic antigen-binding region that specifically binds IL21R.
3. A multispecific antigen binding protein according to embodiment 2, wherein the IL21 polypeptide comprises an amino acid sequence with at least 50, 55, 60, 65, 70, 75, 80, 85, 90, 95, 96, 97, 98, 99, or 100% sequence identity to SEQ ID NO: 38, and preferably has at least one of IL21R agonist activity and affinity for the IL21R.
4. A multispecific antigen binding protein according to embodiment 2 or 3, wherein the IL21 polypeptide is an IL21 mutein that is modified to reduce or enhance affinity for IL21R, relative to a corresponding wild type IL21 polypeptide.
5. A multispecific antigen binding protein according to embodiment 4, wherein the IL21 mutein has reduced affinity for IL21R, relative to a corresponding wild type IL21 polypeptide, and wherein the IL21 mutein has a mutation in one or more amino acids selected from the group consisting of 116, 166, 18, K72, K73, K75, K77, L13, P78, Q12, Q19, R5, R65, R76, R9, S70, S80, V69 and Y23.
6. A multispecific antigen binding protein according to any one of the preceding embodiments, wherein the multispecific antigen binding protein has an IL21R agonist-valency that is higher than one.
7. A multispecific antigen binding protein according to any one of the preceding embodiments, wherein the 4-1BB agonist comprises or consist of at least one 4-1BB ligand (4-1BBL) extracellular domain (ECD) or at least one agonistic antigen-binding region that specifically binds 4-1BB.
8. A multispecific antigen binding protein according to embodiment 6, wherein the 4-1BBL ECD comprises an amino acid sequence with at least 50, 55, 60, 65, 70, 75, 80, 85, 90, 95, 96, 97, 98, 99, or 100% sequence identity to SEQ ID NO: 37, and preferably has at least one of 4-1BB agonist activity and affinity for 4-1BB.
9. A multispecific antigen binding protein according to embodiment 7 or 8, wherein the 4-1BBL ECD is a mutein that is modified to reduce or enhance affinity for 4-1BB, relative to a corresponding wild type 4-1BBL ECD.
10. A multispecific antigen binding protein according to any one of embodiments 7-9, wherein the 4-1BB agonist comprises or consist of a fusion protein comprising three 4-1BBL ECD monomers fused together in a single polypeptide chain, and wherein, optionally, the three 4-1BBL ECD monomers are connected by polypeptide linkers.
11. A multispecific antigen binding protein according to any one of embodiments 7-10, wherein the multispecific antigen binding protein has a 4-1BB agonist-valency that is higher than one.
12. A multispecific antigen binding protein according to any one of the preceding embodiments, wherein the multispecific antigen binding protein comprises an IL21R agonist and a 4-1BB agonist.
13. A multispecific antigen binding protein according to any one of the preceding embodiments, wherein the multispecific antigen binding protein further comprises an NK cell-activating cytokine selected from an IL15 receptor agonist, a type I interferon (IFN-1) agonist, an IL2 receptor agonist, an IL12 receptor agonist and an IL18 receptor agonist.
14. A multispecific antigen binding protein according to any one of the preceding embodiments, wherein the region that has affinity for a surface antigen expressed on NK cells comprises or consists of an immunoglobulin Fc region.
15. A multispecific antigen binding protein according to embodiment 14, wherein the Fc region is a dimeric Fc region.
16. A multispecific antigen binding protein according to embodiment 14 or 15, wherein the Fc region binds to CD16A.
17. A multispecific antigen binding protein according to embodiment 16, wherein the immunoglobulin Fc region is modified to reduce or enhance affinity for CD16A, relative to a corresponding wild-type Fc region.
18. A multispecific antigen binding protein according to any one of the preceding embodiments, wherein the multispecific antigen binding protein further comprises:
   c) at least one antigen-binding region that specifically binds an NK cell activating receptor.
19. A multispecific antigen binding protein according to embodiment 18, wherein the antigen-binding region that specifically binds an NK cell activating receptor is an agonistic antigen-binding region that activates the NK cell receptor.
20. A multispecific antigen binding protein according to any one of embodiments 18-19, wherein the antigen-binding region comprises at least one immunoglobulin variable region.
21. A multispecific antigen binding protein according to embodiment 20, wherein the immunoglobulin variable region comprises or consists of a Fab or an immunoglobulin single variable domain (ISVD).
22. A multispecific antigen binding protein according to any one of embodiments 18-21, wherein the antigen-binding region is a human or humanized antigen-binding region.
23. A multispecific antigen binding protein according to any one of embodiments 18-22, wherein the multispecific antigen binding protein comprises two antigen-binding regions that specifically bind an NK cell activating receptor.
24. A multispecific antigen binding protein according to embodiment 23, wherein the two antigen-binding regions bind the same NK cell activating receptor or at least two different NK cell activating receptors.
25. A multispecific antigen binding protein according to embodiment 24, wherein the two antigen binding regions are identical.
26. A multispecific antigen binding protein according to any one of embodiments 18-25, wherein the NK cell activating receptor is selected from the group consisting of NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CD59, PD-L1, Tim3, CRACC, 4-BB, OX40, CRTAM, CD27, PSGL1, CD96, CD100, CEACAM1, and NTB-A.
27. A multispecific antigen binding protein according to embodiment 26, wherein the antigen-binding region comprises a combination of complementarity-determining regions (CDRs) CDR-H1, CDR-H2, CDR-H3, CDR-L1, CDR-L2 and CDR-L3 selected from the group consisting of:
   a) the CDR-H1 (SEQ ID NO: 24), CDR-H2 (SEQ ID NO: 25) and CDR-H3 (SEQ ID NO: 26) sequences as comprised in SEQ ID NO: 1, and the CDR-L1 (SEQ ID NO: 27), CDR-L2 (SEQ ID NO: 28) and CDR-L3 (SEQ ID NO: 29) sequences as comprised in SEQ ID NO: 2; and b) the CDR-H1, CDR-H2 and CDR-H3 sequences as comprised in SEQ ID NO: 3, and the CDR-L1, CDR-L2 and CDR-L3 sequences as comprised in SEQ ID NO: 20.
28. A multispecific antigen binding protein according to embodiment 27, wherein the antigen-binding region comprises a combination of variable light (V_{L}) and variable heavy (V_{H}) domains selected from the group consisting of:
   a) the V_{H} sequence as comprised in SEQ ID NO: 1 and the V_{L} sequence as comprised in SEQ ID NO: 2; and b) the V_{H} sequence as comprised in SEQ ID NO: 3 and the V_{L} sequence as comprised in SEQ ID NO: 20.
29. A multispecific antigen binding protein according to any one of the preceding embodiments, wherein at least one NK cell-activating cytokine is conjugated to the region that has affinity for a surface antigen expressed on NK cells.
30. A multispecific antigen binding protein according to embodiment 29, wherein the at least one NK cell-activating cytokine forms a single polypeptide chain with at least one polypeptide chain of the region that has affinity for a surface antigen expressed on NK cells, and wherein optionally the NK cell-activating cytokine is linked to the polypeptide chain of the region that has affinity for a surface antigen expressed on NK cells through a flexible linker.
31. A multispecific antigen binding protein according to embodiment 30, wherein the single polypeptide chain comprises in an N- to C-terminal order: i) a first NK cell-activating cytokine, ii) optionally a first flexible linker; iii) the polypeptide chain of the region that has affinity for a surface antigen expressed on NK cells; iv) optionally a second flexible linker; and v) a second NK cell-activating cytokine; and wherein preferably, the first and second NK cell-activating cytokine are different NK cell-activating cytokines.
32. A multispecific antigen binding protein according to any one of embodiments 18-30, wherein the at least one antigen-binding region that specifically binds an NK cell activating receptor is conjugated to the region that has affinity for a surface antigen expressed on NK cells.
33. A multispecific antigen binding protein according to embodiment 32, wherein at least one polypeptide chain in the at least one antigen-binding region that specifically binds an NK cell activating receptor forms a single polypeptide chain with at least one polypeptide chain of the region that has affinity for a surface antigen expressed on NK cells.
34. A multispecific antigen binding protein according to embodiment 33, wherein the single polypeptide chain comprises in an N- to C-terminal order: i) at least one polypeptide chain in the at least one antigen-binding region that specifically binds an NK cell activating receptor; ii) optionally a flexible linker; and iii) the region that has affinity for a surface antigen expressed on NK cells.
35. A multispecific antigen binding protein according to embodiment 33 or 34, wherein the region that has affinity for a surface antigen expressed on NK cells is a dimeric immunoglobulin Fc region, wherein each of the two polypeptide chains of the dimeric Fc region is linked to a CH1 domain, each of which CH1 domains is linked to an immunoglobulin variable region that specifically binds an NK cell activating receptor.
36. A multispecific antigen binding protein according to embodiment 35, wherein the two immunoglobulin variable regions bind the same NK cell activating receptor, or wherein the two immunoglobulin variable regions each bind a different NK cell activating receptor.
37. A multispecific antigen binding protein according to embodiment 35 or 36, wherein the protein comprises a dimeric immunoglobulin Fc region, wherein each of the two Fc polypeptide chains is operably linked to a Fab that specifically binds an NK cell activating receptor.
38. A multispecific antigen binding protein according to any one of embodiments 18-30, wherein at least one NK cell-activating cytokine, is conjugated to the at least one antigen-binding region that specifically binds an NK cell activating receptor, or to the region that has affinity for a surface antigen expressed on NK cells.
39. A multispecific antigen binding protein according to embodiment 37, wherein at least one of the NK cell-activating cytokines forms a single polypeptide chain with at least one of:
   i) at least one polypeptide chain in the at least one antigen-binding region that specifically binds an NK cell activating receptor; and,
   ii) at least one polypeptide chain in the region that has affinity for a surface antigen expressed on NK cells;
wherein optionally, a flexible linker is present between the agonist and the at least one polypeptide chain in the region defined in i) or ii).
40. A multispecific antigen binding protein according to embodiment 38 or 39, wherein at least one of the NK cell-activating cytokines forms a single polypeptide chain with at least one of:
   i) a light chain in at least one of the two Fabs that specifically bind an NK cell activating receptor; and,
   ii) at least one of the two Fc chains in the dimeric immunoglobulin Fc region;
wherein optionally, a flexible linker is present between the agonist and the light chain defined in i) or the Fc chain defined in ii).
41. A multispecific antigen binding protein according to embodiment 40, wherein at least one of the NK cell-activating cytokines is fused to at least one of:
   i) the N-terminus of the light chain in at least one of the two Fabs that specifically bind an NK cell activating receptor, optionally through a flexible linker;
   ii) the C-terminus of the light chain in at least one of the two Fabs that specifically bind an NK cell activating receptor, optionally through a flexible linker;
   iii) the N-terminus of the heavy chain in at least one of the two Fabs that specifically bind an NK cell activating receptor; and,
   iv) the C-terminus of the heavy chain in at least one of the two Fc chains in the dimeric immunoglobulin Fc region, optionally through a flexible linker.
42. A multispecific antigen binding protein according to embodiment 40 or 41, wherein at least one of the NK cell-activating cytokines is present on at least one or on both sides of the immunoglobulin structure.
43. A multispecific antigen binding protein according to any one of embodiments 35-42, wherein the multispecific antigen binding protein is heterodimeric with respect to at least one of i) the antigen-binding regions that specifically bind an NK cell activating receptor; and ii) at least one fused NK cell-activating cytokine, and wherein the dimeric Fc region comprises different first and a second polypeptide chains comprising knob-into-hole modifications promoting association of the first and the second polypeptide chains of the Fc region.
44. A multispecific antigen binding protein according to any one of the preceding embodiments, wherein the multispecific antigen binding protein has at least one biological activity selected from:
   a) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are not brought into contact with the multispecific antigen binding protein; and,
   b) the multispecific antigen binding protein cause an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are brought into contact with a conventional human IgG1 monoclonal antibody that has same NK cell activating receptor-specific antigen-binding regions as the multispecific antigen binding protein.
45. A multispecific antigen binding protein according to any one of the preceding embodiments, wherein ex vivo expansion of donor NK cells by co-culturing with the multispecific antigen binding protein as described herein, produces a population of expanded NK cells having one or more features selected from:
   a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
   b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of NK cells obtained upon ex vivo expansion in the presence of FC21 feeder cells;
   c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on expanded NK cells obtained by ex vivo expansion by co-culturing with irradiated FC21 feeder cells;
   d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold of the secretion of the cytokine by expanded NK cells obtained by ex vivo expansion by co-culturing with irradiated FC21 feeder cells; and,
   e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of expanded NK cells obtained by ex vivo expansion by co-culturing with irradiated FC21 feeder cells.
46. A pharmaceutical composition comprising a multispecific antigen binding protein according to any one of the preceding embodiments, and a pharmaceutically acceptable carrier.
47. An ex vivo method for expansion of NK cells, the method comprising the step of contacting an NK cell with a multispecific antigen binding protein according to any one of embodiments 1-45, or with a composition according to embodiment 46, and wherein preferably, the expanded NK cells have one or more features selected from:
   a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
   b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells;
   c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells;
   d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and,
   e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells.
48. A multispecific antigen binding protein according to any one of embodiments 1-45, a composition according to embodiment 46, or an ex vivo expanded NK cell obtained in a method according to embodiment 47, optionally in combination with the multispecific antigen binding protein, for use as a medicament.
49. A multispecific antigen binding protein according to any one of embodiments 1-45, a composition according to embodiment 46, or an ex vivo expanded NK cell obtained in a method according to embodiment 47, optionally in combination with the multispecific antigen binding protein, for use in the treatment of a cancer.
50. A multispecific antigen binding protein according to any one of embodiments 1-45, a composition according to embodiment 46, for use in the treatment of a cancer, wherein the multispecific antigen binding protein or the composition is used in combination with an adoptive transfer of immune cells, wherein preferably the immune cells are selected from T cells and NK cells.
48. A multispecific antigen binding protein according to any one of embodiments 1-45, a composition according to embodiment 46, for a use according to claim 49 or 50, wherein at least one of:
   a) the multispecific antigen binding protein is administered as a neoadjuvant therapy before a primary therapy comprising at least one of surgery and radiation therapy of the cancer; and,
   b) the multispecific antigen binding protein is administered as an adjuvant therapy after a primary therapy comprising at least one of surgery and radiation therapy of the cancer.
51. A method for enhancing anti-tumor activity of an NK cell in a subject, the method comprising the step of administering to the subject a multispecific antigen binding protein according to any one of embodiments 1-45, a composition according to embodiment 46, an ex vivo expanded NK cell obtained in a method according to embodiment 47, optionally, in combination with the multispecific antigen binding protein, or a combination of the multispecific antigen binding protein and an immune cell selected from T cells and NK cells.
52. The method of 51, wherein the subject has cancer.
53. The method of claim 52, wherein at least one of:
   a) the multispecific antigen binding protein is administered to the subject as a neoadjuvant therapy before a primary therapy comprising at least one of surgery and radiation therapy of the cancer; and,
   b) the multispecific antigen binding protein is administered to the subject as an adjuvant therapy after a primary therapy comprising at least one of surgery and radiation therapy of the cancer.
54. A nucleic acid molecule comprising one or more nucleotide sequences encoding a polypeptide chain of a multispecific antigen binding protein according to any one of embodiments 1-45.
55. A nucleic acid molecule according to embodiment 54, wherein the one or more nucleotide sequences are operably linked to regulatory sequences for expression of the one or more polypeptide chains in a host cell.
56. A host cell comprising a nucleic acid molecule according to embodiment 54 or 55.
57. A method for producing a multispecific antigen binding protein according to any one of embodiments 1-45, the method comprising culturing a host cell according to embodiment 56 such that one or more nucleotide sequences are expressed, and the multispecific antigen binding protein is produced.
58. A method according to embodiment 57, further comprising the steps of: recovery of the multispecific antigen binding protein; and, optionally, formulation of the multispecific antigen binding protein with a pharmaceutically acceptable carrier.

1. A multispecific antigen binding protein comprising:
a) at least two NK cell-activating cytokines comprising:
i) an interleukin 21 receptor (IL21R) agonist; and,
ii) a 4-1BB agonist; and,
b) a region that has affinity for a surface antigen expressed on natural killer (NK) cells, wherein the region comprises or consists of an immunoglobulin Fc region,
wherein the IL21R agonist comprises or consist of an IL21 polypeptide or an agonistic antigen-binding region that specifically binds IL21R,
and wherein the 4-1BB agonist comprises or consist of at least one 4-1BB ligand (4-1BBL) extracellular domain (ECD) or at least one agonistic antigen-binding region that specifically binds 4-1BB, wherein preferably, the 4-1BB agonist comprises or consist of a fusion protein comprising three 4-1BBL ECD monomers fused together in a single polypeptide chain, and wherein, optionally, the three 4-1BBL ECD monomers are connected by polypeptide linkers.

2. A multispecific antigen binding protein according to claim 1, wherein the IL21 polypeptide comprises an amino acid sequence with at least 70% sequence identity to SEQ ID NO: 38 and having IL21R agonist activity, and wherein the 4-1BBL ECD comprises an amino acid sequence with at least 70% sequence identity to SEQ ID NO: 37 and having 4-1BB agonist activity.

3. A multispecific antigen binding protein according to claim 1, wherein the Fc region is a dimeric Fc region that binds to CD16A, and preferably activates the NK cell.

4. A multispecific antigen binding protein according to claim 1, wherein the multispecific antigen binding protein further comprises:
c) at least one antigen-binding region that specifically binds an NK cell activating receptor,
wherein preferably, the antigen-binding region that specifically binds an NK cell activating receptor is an agonistic antigen-binding region that activates the NK cell receptor, and wherein preferably, the NK cell activating receptor is selected from the group consisting of NKp46, NKp30, NKG2D, CD16A, SLAMF7, NKp44, CD94-NKG2C/E, KIR2DS1, KIR2DS3, KIR2DS4, KIR2DS5, KIR2DS2, KIR2DL4, KIR3DS1, CD160, NKp80, DNAM1, 2B4, CRACC, 4-BB, OX40, CRTAM, CD27, PSGL1, CD96, CD100, CEACAM1, CD59, PD-L1, Tim3 and NTB-A.

5. A multispecific antigen binding protein according to claim 1, wherein at least one NK cell-activating cytokine is conjugated to the region that has affinity for a surface antigen expressed on NK cells, wherein preferably, the at least one NK cell-activating cytokine forms a single polypeptide chain with at least one polypeptide chain of the region that has affinity for a surface antigen expressed on NK cells, and wherein optionally the NK cell-activating cytokine is linked to the polypeptide chain of the region that has affinity for a surface antigen expressed on NK cells through a flexible linker, and, wherein more preferably, the single polypeptide chain comprises in an N- to C-terminal order: i) a first NK cell-activating cytokine, ii) optionally a first flexible linker; iii) the polypeptide chain of the region that has affinity for a surface antigen expressed on NK cells; iv) optionally a second flexible linker; and v) a second NK cell-activating cytokine; and wherein preferably, the first and second NK cell-activating cytokine are different NK cell-activating cytokines.

6. A multispecific antigen binding protein according to claim 5, wherein the at least one antigen-binding region that specifically binds an NK cell activating receptor is conjugated to the region that has affinity for a surface antigen expressed on NK cells,
wherein preferably, at least one polypeptide chain in the at least one antigen-binding region that specifically binds an NK cell activating receptor forms a single polypeptide chain with at least one polypeptide chain of the region that has affinity for a surface antigen expressed on NK cells,
wherein more preferably, the single polypeptide chain comprises in an N- to C-terminal order: i) at least one polypeptide chain in the at least one antigen-binding region that specifically binds an NK cell activating receptor; ii) optionally a flexible linker; and iii) the region that has affinity for a surface antigen expressed on NK cells,
wherein still more preferably, the region that has affinity for a surface antigen expressed on NK cells is a dimeric immunoglobulin Fc region, wherein each of the two polypeptide chains of the dimeric Fc region is linked to a CH1 domain, each of which CH1 domains is linked to an immunoglobulin variable region that specifically binds an NK cell activating receptor, wherein most preferably, the protein comprises a dimeric immunoglobulin Fc region, wherein each of the two Fc polypeptide chains is operably linked to a Fab that specifically binds an NK cell activating receptor.

7. A multispecific antigen binding protein according to claim 5, wherein at least one NK cell-activating cytokine, is conjugated to the at least one antigen-binding region that specifically binds an NK cell activating receptor, or to the region that has affinity for a surface antigen expressed on NK cells,
wherein preferably, at least one of the NK cell-activating cytokines forms a single polypeptide chain with at least one of:
i) at least one polypeptide chain in the at least one antigen-binding region that specifically binds an NK cell activating receptor; and,
ii) at least one polypeptide chain in the region that has affinity for a surface antigen expressed on NK cells;
wherein optionally, a flexible linker is present between the agonist and the at least one polypeptide chain in the region defined in i) or ii),
wherein more preferably, at least one of the NK cell-activating cytokines forms a single polypeptide chain with at least one of:
i) a light chain in at least one of the two Fabs that specifically bind an NK cell activating receptor; and,
ii) at least one of the two Fc chains in the dimeric immunoglobulin Fc region;
wherein optionally, a flexible linker is present between the agonist and the light chain defined in i) or the Fc chain defined in ii),
wherein still more preferably, at least one of the NK cell-activating cytokines is fused to at least one of:
i) the N-terminus of the light chain in at least one of the two Fabs that specifically bind an NK cell activating receptor, optionally through a flexible linker;
ii) the C-terminus of the light chain in at least one of the two Fabs that specifically bind an NK cell activating receptor, optionally through a flexible linker;
iii) the N-terminus of the heavy chain in at least one of the two Fabs that specifically bind an NK cell activating receptor; and,
iv) the C-terminus of the heavy chain in at least one of the two Fc chains in the dimeric immunoglobulin Fc region, optionally through a flexible linker,
and wherein most preferably, at least one of the NK cell-activating cytokines is present on at least one or on both sides of the immunoglobulin structure.

8. A multispecific antigen binding protein according to claim 5, wherein the multispecific antigen binding protein is heterodimeric with respect to at least one of i) the antigen-binding regions that specifically bind an NK cell activating receptor; and ii) at least one fused NK cell-activating cytokine, and wherein the dimeric Fc region comprises different first and a second polypeptide chains comprising knob-into-hole modifications promoting association of the first and the second polypeptide chains of the Fc region.

9. A multispecific antigen binding protein according to claim 1, wherein the multispecific antigen binding protein has at least one biological activity selected from:
a) the multispecific antigen binding protein causes an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are not brought into contact with the multispecific antigen binding protein; and,
b) the multispecific antigen binding protein cause an increase in at least one NK cell activity selected from CD107a degranulation, CD107 or CD69 expression, IFNy production, NK cell proliferation and NK cell cytotoxicity, whereby preferably, the increase is at least a factor 0.1 higher as compared to the increase achieved with the same effector:target cell ratio, with the same NK cells and target cells that are brought into contact with a conventional human IgG1 monoclonal antibody that has same NK cell activating receptor-specific antigen-binding regions as the multispecific antigen binding protein.

10. A multispecific antigen binding protein according to claim 1, wherein ex vivo expansion of donor NK cells by co-culturing with the multispecific antigen binding protein as described herein, produces a population of expanded NK cells having one or more features selected from:
a) the fold expansion of the expanded NK cells is at least 0.001, 0.002, 0.005, 0.01, 0.02, 0.05, 0.1, 0.2, 0.5, 1.0, 2.0 or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0 or 5.0 fold of the percentage telomere length increase of NK cells obtained upon ex vivo expansion in the presence of FC21 feeder cells;
c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0 or 5.0 fold of the expression level on expanded NK cells obtained by ex vivo expansion by co-culturing with irradiated FC21 feeder cells;
d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0 or 5.0 fold of the secretion of the cytokine by expanded NK cells obtained by ex vivo expansion by co-culturing with irradiated FC21 feeder cells; and,
e) the cytotoxicity of the expanded NK cells is at least 0.001, 0.002, 0.005, 0.01, 0.02, 0.05, 0.1, 0.2, 0.5, 1.0, 2.0 or 5.0 fold of the cytotoxicity of expanded NK cells obtained by ex vivo expansion by co-culturing with irradiated FC21 feeder cells.

11. (canceled)

12. An ex vivo method for expansion of NK cells, the method comprising the step of contacting an NK cell with a multispecific antigen binding protein according to claim 1, and wherein preferably, the expanded NK cells have one or more features selected from:
a) the fold expansion of the expanded NK cells is at least 0.001, 0.002, 0.005, 0.01, 0.02, 0.05, 0.1, 0.2, 0.5, 1.0, 2.0, or 5.0 fold of the fold expansion of expanded NK cells obtained by ex vivo expansion by co-culturing with irradiated K562 feeder cells modified to express membrane bound IL-21 (mbIL-21) and 4-1BB ligand (FC21 feeder cells);
b) the telomere length of the expanded NK cells is increased by at least 10, 15, 20, 25, 30, 35, 40, 45, 50 or 55% as compared to the telomere length of fresh NK cells, preferably, the percentage telomere length increase of the expanded NK cells as compared to the telomere length of fresh NK cells, is at least 0.001, 0.002, 0.005, 0.01, 0.02, 0.05, 0.1, 0.2, 0.5, 1.0, 2.0, or 5.0 fold of the percentage telomere length increase of NK cells obtained upon ex vivo expansion in the presence of FC21 feeder cells;
c) the expression level of at least one NK cell activating receptor selected from NKG2D, NKp30, NKp44, NKp46 and CD16 on the expanded NK cells is at least 0.001, 0.002, 0.005, 0.01, 0.02, 0.05, 0.1, 0.2, 0.5, 1.0, 2.0, or 5.0 fold of the expression level on expanded NK cells obtained by ex vivo expansion by co-culturing with irradiated FC21 feeder cells;
d) the secretion of at least one cytokine of TNF-α, IFN-γ and IL-6 by the expanded NK cells is at least 0.001, 0.002, 0.005, 0.01, 0.02, 0.05, 0.1, 0.2, 0.5, 1.0, 2.0, or 5.0 fold of the secretion of the cytokine by expanded NK cells obtained by ex vivo expansion by co-culturing with irradiated FC21 feeder cells; and,
e) the cytotoxicity of the expanded NK cells is at least 0.001, 0.002, 0.005, 0.01, 0.02, 0.05, 0.1, 0.2, 0.5, 1.0, 2.0, or 5.0 fold of the cytotoxicity of expanded NK cells obtained by ex vivo expansion by co-culturing with irradiated FC21 feeder cells.

13.-15. (canceled)

16. A method for treating cancer in a subject in need thereof, the method comprising administering to the subject a multispecific antigen binding protein according to claim 1.

17. The method according to claim 16, wherein the method comprises the administration of an ex vivo expanded NK cell obtained in a method according to claim 12.

18. The method according to claim 16, wherein the multispecific antigen binding protein is administered in combination with an adoptive transfer of immune cells, wherein preferably the immune cells are selected from T cells and NK cells.
